(19) United States
(12) Patent Application Publication
(10) Pub. No.: US 2024/0425435 A1
(43) Pub. Date: Dec. 26, 2024

(54) AN INTEGRATED PROCESS FOR PRODUCING TRIFLUOROIODOMETHANE

(71) Applicant: Honeywell International Inc., Charlotte, NC (US)

(72) Inventors: Haluk Kopkalli, Staten Island, NY (US); Haiyou Wang, Amherst, NY (US); Gustavo Cerri, Parsippany, NJ (US); Selma Bektesevic, Williamsville, NY (US); Yuon Chiu, Denville, NJ (US); Christian Jungong, Depew, NY (US); Richard D. Horwath, Richmond, VA (US); Daniel C. Merkel, Orchard Park, NY (US); Jennifer W. McClaine, Branchburg, NJ (US); Terris Yang, East Amherst, NY (US); Richard Wilcox, West Caldwell, NJ (US); Joshua Close, Cheektowaga, NY (US); Rajendar Mallepally, Charlotte, NC (US)

(21) Appl. No.: 18/570,415

(22) PCT Filed: Jul. 11, 2022
(86) PCT No.: PCT/US2022/073603

§ 371 Date: Dec. 14, 2023

Related U.S. Application Data
(60) Provisional application No. 63/222,819, filed on Jul. 16, 2021.

(51) Int. Cl. C07C 17/363; B01J 27/224

(52) U.S. Cl. CPC C07C 17/363; B01J 27/224

(57) ABSTRACT

The present disclosure provides an integrated process for producing trifluoroiodomethane (CF₃I), in three steps: a) reacting a first reactant stream comprising hydrogen (H₂) and iodine (I₂) in the presence of a first catalyst to produce a first product stream comprising hydrogen iodide (HI); (b) reacting the first product stream with a second reactant stream comprising trifluoroacetyl chloride (TFAC) in the presence of a second catalyst to produce an intermediate product stream comprising trifluoroacetyl iodide (TFAI); and (c) reacting the intermediate product stream to produce a final product stream comprising trifluoroiodomethane. (CF₃I).

### CROSS-REFERENCE TO RELATED APPLICATION

This application claims priority to Provisional Application No. 63/222,819, filed Jul. 16, 2021, which is herein incorporated by reference in its entirety.

### FIELD

The present disclosure relates to processes for producing trifluoroiodomethane (CF₃I). Specifically, the present disclosure relates to an integrated process to produce trifluoroiodomethane.

### BACKGROUND

Trifluoroiodomethane (CF₃I), also known as perfluoromethyliodide, trifluoromethyl iodide, or iodotrifluoromethane, is a useful compound in commercial applications, as a refrigerant or a fire suppression agent, for example. Trifluoroiodomethane is a low global warming potential molecule with almost no ozone depletion potential. Trifluoroiodomethane can replace more environmentally damaging materials.

Methods of preparing trifluoroiodomethane are known. For example, U.S. Pat. No. 7,196,236 (Mukhopadhyay et al.) discloses a catalytic process for producing trifluoroiodomethane using reactants comprising a source of iodine, at least a stoichiometric amount of oxygen, and a reactant CF₃R, where R is selected from the group consisting of —COOH, —COX, —CHO, —COOR₂, AND —SO₂X, where R₂ is alkyl group and X is a chlorine, bromine, or iodine. Hydrogen iodide, which may be produced by the reaction, can be oxidized by the at least a stoichiometric amount of oxygen, producing water and iodine for economic recycling.

In another example, U.S. Pat. No. 7,132,578 (Mukhopadhyay et al.) also discloses a catalytic, one-step process for producing trifluoroiodomethane from trifluoroacetyl chloride. However, the source of iodine is iodine fluoride (IF). In contrast to hydrogen iodide, iodine fluoride is relatively unstable, decomposing above 0° C. to I₂ and IF₅. Iodine fluoride may also not be available in commercially useful quantities.

In another example, U.S. Pat. No. 10,752,565 (Nair et al.) a gas-phase process for producing trifluoroiodomethane is disclosed. The process comprises providing a reactant stream comprising hydrogen iodide and trifluoroacetyl halide selected from the group consisting of trifluoroacetyl chloride, trifluoroacetyl fluoride, trifluoroacetyl bromide, and combinations thereof, and reacting the reactant stream in the presence of a catalyst at a temperature from about 200° C. to about 600° C. to produce a product stream comprising the trifluoroiodomethane.

There is a need to develop a more efficient process that may be scaled to produce commercial quantities of trifluoroiodomethane from relatively inexpensive raw materials.

### SUMMARY

The present disclosure provides an integrated process for producing trifluoroiodomethane (CF₃I).

According to one embodiment, the present disclosure provides a process for producing trifluoroiodomethane (CF₃I), the process including: (a) providing a first reactant stream comprising hydrogen iodide (HI); (b) reacting the first reactant stream with a second reactant stream comprising trifluoroacetyl chloride (TFAC) to produce an intermediate product stream comprising trifluoroacetyl iodide (TFAI); and (c) reacting the intermediate product stream to produce a final product stream comprising trifluoroiodomethane (CF₃I).

### BRIEF DESCRIPTION OF THE DRAWINGS

FIG. 1 is a process flow diagram for a first step of the present integrated process, including the production of hydrogen iodide (HI) from hydrogen (H₂) and iodine (I₂).

FIG. 2 is process flow diagram of a second step of the present integrated process, including the production of trifluoroacetyl iodide (TFAI) from trifluoroacetyl chloride (TFAC) and hydrogen iodide (HI).

FIG. 2A is process flow diagram of an alternative embodiment of a second step of the present integrated process, including the production of trifluoroacetyl iodide (TFAI) from trifluoroacetyl chloride (TFAC) and hydrogen iodide (HI).

FIG. 3 is a process flow diagram of a third step of the present integrated process, including the production of trifluoroiodomethane (CF₃I) from trifluoroacetyl iodide (TFAI).

FIG. 4 is an experimental set up for a trifluoroacetyl iodide (TFAI) vaporizer/superheater/pyrolysis reactor as described in Example 8.

FIG. 5 is a process flow diagram for deacidifying crude CF3I with caustic solution and drying wet, acid-free crude CF3I with concentrated sulfuric acid as described in Example 27b.

FIG. 6 is a process flow diagram for producing an unreacted TFAI stream for recycle, a purified CF3I product, and a CO waste stream as described in Example 29.

### DETAILED DESCRIPTION

The present disclosure provides an integrated process for producing trifluoroiodomethane (CF₃I) according to the overall reaction scheme below:

H₂+I₂→2HI Eq. 1:

TFAC+HI→TFAI+HCl Eq. 2:

TFAI→CF₃I+CO Eq. 3:

### 1. Formation of Hydrogen Iodide (HI)

As disclosed herein, in a first reaction step for the integrated process to produce CF3I, hydrogen (H₂) may be reacted with iodine (I₂) to form hydrogen iodide (HI). The HI may be anhydrous hydrogen iodide, which is produced from a reactant stream comprising hydrogen and iodine. The reactant stream may consist essentially of hydrogen and iodine. The reactant stream may consist of hydrogen and iodine.

The production of anhydrous HI (Eq. 1) is described in greater detail below. Alternatively, HI may be produced by other means or purchased for use in the process of the invention. HI may be further purified before being fed to the integrated process to manufacture trifluoroiodomethane (CF₃I) from trifluoroacetyl chloride (TFAC) and HI.

The process includes providing a vapor-phase reactant stream comprising hydrogen and iodine and reacting the reactant stream in the presence of a catalyst to produce a product stream comprising hydrogen iodide. The catalyst includes at least one selected from the group of nickel, nickel iodide (NiI₂), cobalt, cobalt iodide (CoI₂), iron, iron iodide (FeI₂ or FeI₃), nickel oxide, cobalt oxide, and iron oxide. The catalyst may be supported on a support.

The process includes the steps of reacting hydrogen and iodine in the vapor phase in the presence of a catalyst to produce a product stream comprising HI, unreacted iodine and unreacted hydrogen, removing at least some of the unreacted iodine from the product stream by cooling the product stream to form solid iodine, producing liquid iodine from the solid iodine, and recycling the liquified iodine to the reacting step. The solid iodine forms in a first iodine removal vessel or a second iodine removal vessel. The liquid iodine is produced from the solid iodine by heating the first iodine removal vessel to liquefy the solid iodine when cooling the product stream through the second iodine removal vessel or heating the second iodine removal vessel to liquefy the solid iodine when cooling the product stream through the first iodine removal vessel. Unreacted hydrogen is recycled to the reacting step. The catalyst includes at least one selected from the group of nickel, nickel iodide (NiI₂), cobalt, cobalt iodide (CoI₂), iron, iron iodide (FeI₂ or FeI₃), nickel oxide, cobalt oxide, and iron oxide. The catalyst may be supported on a support.

The catalyst may be supplied in a passivated form and may then be activated. Additionally, the catalyst may be converted from one species to another over the course of the reaction. For example, metallic nickel on a support may be converted in situ into nickel iodide (NiI₂). Metallic nickel supported on inert materials may be commercially available at various loadings of the nickel metal. When supplied, the nickel supported on inert material is in a passivated form and may need to be activated in hydrogen gas to expose the metallic nickel phase, before iodine vapors are supplied to convert the metallic nickel phase into NiI₂. Alternatively, catalysts that may be prepared in situ like NiI₂, or may be supplied in a ready-made form, by preparing the catalyst through impregnating, pore filling, precipitation, and/or adsorption onto the support.

The catalyst may be deliquescent and when exposed to ambient conditions it may absorb moisture and dissolve in its own water of hydration. Therefore, whether the catalyst is prepared in situ or externally, it may be desired to use the catalyst in anhydrous conditions, as exposure of the catalyst to moisture may result in the formation of a hydrated complex. The formation of hydrated complexes may be associated with significant agglomeration and loss of catalytic activity. The deactivated catalyst may be regenerated by drying in hot inert gas, followed by successive repeated cycles of reduction in hydrogen gas or other suitable reducing agents and oxidation in oxygen gas or other oxidizing agents.

The catalyst may also be regenerated after a period of time. The regenerated catalyst may have reduced catalyst particle size and may have increased catalytic activity compared to spent and/or agglomerated catalyst. For example, a fresh catalyst may have a first particle size, and a spent catalyst may have a second particle size larger than the first particle size. The spent catalyst may also agglomerate when exposed to ambient conditions forming a third particle size larger than the second particle size. The agglomerated catalyst may be dried and chemically reduced to reduce the particle size and increase catalytic activity. The catalyst may undergo multiple rounds of reduction, oxidation, and drying to further reduce particle size and/or increase catalytic activity, thereby making a regenerated catalyst with a fourth particle size. The reduction may be carried out with hydrogen gas, and the oxidation may be carried out with oxygen gas. Other reducing and oxidizing agents may also be used. Suitable non-limiting examples of reducing agents include hydrogen, carbon monoxide (CO), ammonia (NH₃), alkanes such as methane (CH₄). Suitable non-limiting examples of oxidizing agents include oxygen (O₂), ozone (O₃), nitrogen oxides (NO₂, N₂O).

The hydrogen and iodine are anhydrous. It is preferred that there be as little water in the reactant stream as possible because the presence of moisture results in the formation of hydroiodic acid, which is corrosive and can be detrimental to equipment and process lines. In addition, recovery of the HI from the hydroiodic acid adds to the manufacturing costs.

The hydrogen is substantially free of water, including any water by weight in an amount less than about 500 ppm, about 300 ppm, about 200 ppm, about 100 ppm, about 50 ppm, about 30 ppm, about 20 ppm, about 10 ppm, about 5 ppm, about 2 ppm, or about 1 ppm, or less than any value defined between any two of the foregoing values. Preferably, the hydrogen comprises any water by weight in an amount less than about 50 ppm. More preferably, the hydrogen comprises any water by weight in an amount less than about 10 ppm. Most preferably, the hydrogen comprises any water by weight in an amount less than about 5 ppm.

The iodine is also substantially free of water, including any water by weight in an amount less than about 3000 ppm, about 2000 ppm, about 1000 ppm, about 500 ppm, about 300 ppm, about 200 ppm, about 100 ppm, about 50 ppm, about 30 ppm, about 20 ppm, or about 10 ppm, or less than any value defined between any two of the foregoing values. Preferably, the iodine comprises any water by weight in an amount less than about 100 ppm. More preferably, the iodine comprises any water by weight in an amount less than about 30 ppm. Most preferably, the iodine comprises any water by weight in an amount less than about 10 ppm.

Elemental iodine in solid form is commercially available from, for example, SQM, Santiago, Chile, or Kanto Natural Gas Development Co., Ltd, Chiba, Japan. Hydrogen in compressed gas form is commercially available from, for example, Airgas, Radnor, PA.

The reactant stream and the catalyst may be pre-heated to a reaction temperature. The reaction temperature may be as low as about 150° C., about 200° C., about 250° C., about 280° C., about 290° C., about 300° C., about 310° C., or about 320° C., or to a reaction temperature as high as about 330° C., about 340° C., about 350° C., about 360° C., about 380° C., about 400° C., about 450° C., about 500° C., about 550° C., or about 600° C., or within any range defined between any two of the foregoing values, such as about 150° C. to about 600° C., about 200° C. to about 550° C., about 250° C. to about 500° C., about 280° C. to about 450° C., about 290° C. to about 400° C., about 300° C. to about 380° C., about 310° C. to about 360° C., about 320° C. to about 350° C., or about 320° C. to about 340° C., for example. Preferably, the reaction temperature is from about 200° C. to about 500° C. More preferably, the reaction temperature is from about 300° C. to about 400° C. Most preferably, the reaction temperature is from about 300° C. to about 350° C.

An operating pressure of the reactor may be as low as about 0 psig, about 10 psig, about 20 psig, about 40 psig, about 100 psig, about 125 psig, about 150 psig, about 175 psig, or about as high as 200 psig, about 250 psig, about 300 psig, about 400 psig, about 500 psig, about 600 psig, or any range defined between any two of the foregoing values, such as about 0 psig to 600 psig, about 10 psig to about 500 psig, about 20 psig to about 400 psig, about 40 psig to about 300 psig, about 100 psig to about 250 psig, about 150 psig to about 175 psig, or about 0 psig to about 175 psig, for example. Preferably, the operating pressure of the reactor is from about 5 psig to about 300 psig. More preferably, the operating pressure of the reactor is from about 5 psig to about 150 psig. Most preferably, the operating pressure of the reactor is from about 5 psig to about 120 psig.

In the reactant stream, a mole ratio of hydrogen to iodine may be as low as about 1:1, about 1.5:1, about 2:1, about 2.5:1, about 2.7:1, or about 3:1, or as high as about 4:1, about 5:1, about 6:1, about 7:1, about 8:1, about 9:1, or about 10:1, or within any range defined between any two of the foregoing values, such as about 1:1 to about 10:1, about 2:1 to about 8:1, about 3:1 to about 6:1, about 2:1 to about 5:1, about 2:1 to about 3:1, about 2.5:1 to about 3:1, or about 2.7:1 to about 3.0:1, for example. Preferably, the mole ratio of hydrogen to iodine is from about 2:1 to about 9:1. More preferably, the mole ratio of hydrogen to iodine is from about 2.5:1 to about 8:1. Most preferably, the mole ratio of hydrogen to iodine is from about 2.5:1 to 6:1.

The reactant stream may be in contact with the catalyst for a contact time as short as about 0.1 second, about 2 seconds, about 4 seconds, about 6 seconds, about 8 seconds, about 10 seconds, about 15 seconds, about 20 seconds, about 25 seconds, or about 30 seconds, or as long as about 40 seconds, about 50 seconds, about 60 seconds, about 70 seconds, about 80 seconds, about 100 seconds, about 120 seconds, about 200 seconds, or about 1,800 seconds or within any range defined between any two of the foregoing values, such as about 0.1 seconds to about 1,800 seconds, about 2 seconds to about 120 seconds, about 4 second to about 100 seconds, about 6 seconds to about 80 seconds, about 8 seconds to about 70 seconds, about 10 seconds to about 60 seconds, about 15 seconds to about 50 seconds, about 20 seconds to about 40 seconds, about 20 seconds to about 30 seconds, about 10 seconds to about 20 seconds, or about 100 seconds to about 120 seconds, for example. Preferably, the reactant stream is in contact with the catalyst for a contact time from about 2 seconds to about 200 seconds.

In another embodiment, the process includes the steps of reacting hydrogen and iodine in the vapor phase in the presence of a catalyst to produce a product stream comprising hydrogen iodide, unreacted iodine and unreacted hydrogen, optionally directing the reactor effluent to a compressor to increase the pressure of the crude hydrogen iodide product to facilitate the recovery of the hydrogen and the hydrogen iodide in a distillation column (with a number of theoretical stages ranging from two to many) whereby a stream comprising hydrogen and HI is recycled for further reaction and a liquid stream comprising hydrogen iodide and iodine is further subjected to separation to recover a stream rich in iodine for recycle to the reactor and a stream rich in HI is returned to the distillation column. A portion of purified HI not returned to the distillation column may be used in the step described in Eq. 2 shown above. To control build-up of moisture in the system, one or more of the liquid stream comprising hydrogen iodide and iodine, recycle stream rich in iodine or stream rich in HI may be passed through an adsorbent bed to selectively adsorb water. A purge stream from this step which comprises HI and hydrogen may optionally be sent to the step described in Eq. 2 shown above for further recovery of HI. Multiple side streams (by way of side draws from the distillation column) may be utililized for the purpose of removing iodine, hydrogen or water from the system described.

In yet another embodiment, the process includes the steps of reacting hydrogen and iodine in the vapor phase in the presence of a catalyst to produce a product stream comprising hydrogen iodide, unreacted iodine and unreacted hydrogen, directing the reactor effluent to a distillation column (with number of theoretical stages ranging from 2 to many) whereby a stream comprising hydrogen and HI is recycled for further reaction and a liquid stream comprising hydrogen iodide and iodine is further subjected to separation to recover a stream rich in iodine for recycle to the reactor and a stream rich in HI is returned to the distillation column. A portion of purified HI not returned to the distillation column may be used in the step described in Eq. 2 shown above. To control build-up of moisture in the system, one or more of the liquid stream comprising hydrogen iodide and iodine, recycle stream rich in iodine or stream rich in HI may be passed through an adsorbent bed to selectively adsorb water. A purge stream from this step which comprises HI and hydrogen may optionally be sent to the step described in Eq. 2 shown above for further recovery of HI. Multiple side streams (by way of side draws from the distillation column) may be utililized for the purpose of removing iodine, hydrogen or water from the system described.

The present disclosure provides a method to recover residual unreacted iodine from this step, if desired. In one such method, a stream including iodine vapor and at least one of an inert gas and water vapor may be contacted with an alkaline solution to form an iodide salt which may be recovered.

In another alternative, a stream including iodine vapor and water vapor may be contacted with an adsorbent to selectively adsorb water from the stream, providing a stream of iodine that may be substantially free of water and suitable for recovery or recycling.

In yet another alternative, a stream including an inert gas, water vapor, and iodine vapor may be contacted with a concentrated acid to absorb the water vapor from the stream to provide a stream of iodine that may be substantially free of water and suitable for recovery or recycling.

A further alternative is a method of recovering iodine including providing a stream including iodine vapor and water vapor, and desublimating or condensing the iodine vapor to form solid or liquid iodine, which may be recovered or recycled.

Finally, still another method of recovering iodine includes providing a stream including iodine vapor and at least one of: an inert gas and water vapor, and contacting the stream with a material to condense or desublimate the iodine vapor from the stream as the material absorbs latent heat through a phase change of the material and absorbs sensible heat.

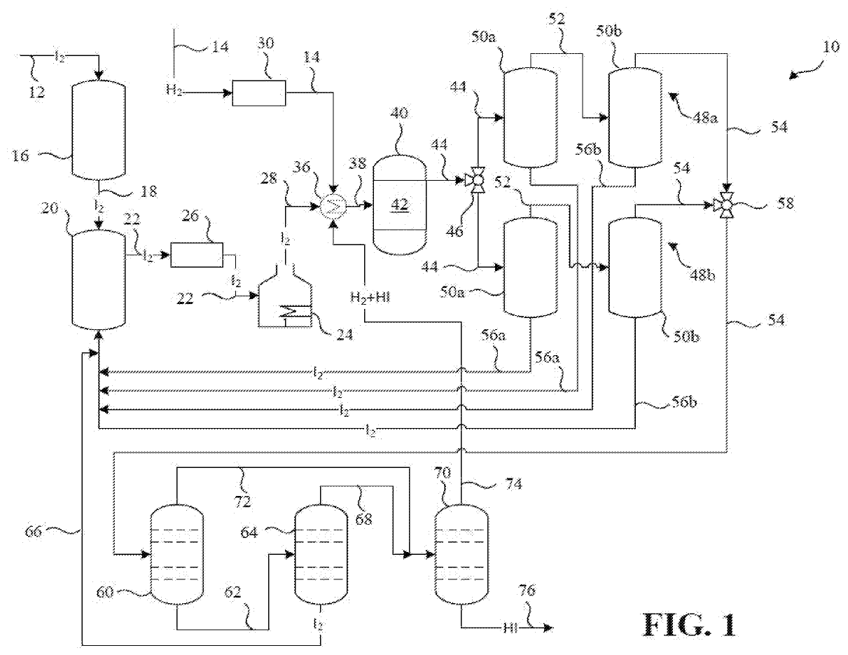

FIG. 1 shows an example of one method to produce anhydrous HI from hydrogen and iodine. As shown in FIG. 1, the process 10 may include material flows of solid iodine 12 and hydrogen gas 14. The solid iodine 12 may be continuously or intermittently added to a solid storage tank 16. A flow of solid iodine 18 may be transferred, continuously or intermittently, by a solid conveying system (not shown) or by gravity from the solid storage tank 16 to an iodine liquefier 20 where the solid iodine is heated to above its melting point but below its boiling point to maintain a level of liquid iodine in the iodine liquefier 20. Although only one liquefier 20 is shown, it is understood that multiple liquefiers 20 may be used in a parallel arrangement. Liquid iodine 22 may then flow from the iodine liquefier 20 to an iodine vaporizer 24. The iodine liquefier 20 may be pressurized by an inert gas to drive the flow of liquid iodine 22. The inert gas may include nitrogen, argon, or helium, or mixtures thereof, for example. Alternatively, or additionally, the flow of liquid iodine 22 may be driven by a pump (not shown). The flow rate of the liquid iodine 22 may be controlled by a liquid flow controller 26. In the iodine vaporizer 24, the iodine may be heated to above its boiling point to form a flow of iodine vapor 28. The flow rate of the hydrogen 14 may be controlled by a gas flow controller 30. The flow of iodine vapor 28 and the flow of hydrogen 14 are provided to a superheater 36 and heated to the reaction temperature to form a reactant stream 38. The reactant stream 38 is provided to a reactor 40. The reactant stream 38 reacts in the presence of a catalyst 42 contained within the reactor 40 to produce a product stream 44. The catalyst 42 may be any of the catalysts described herein. The product stream 44 may include hydrogen iodide, unreacted iodine, unreacted hydrogen and trace amounts of water and other impurities.

The product stream 44 may be provided to an upstream valve 46. The upstream valve 46 may direct the product stream 44 to an iodine removal step. Alternatively, the product stream 44 may pass through a cooler (not shown) to remove some of the heat before being directed to the iodine removal step. In the iodine removal step, a first iodine removal train 48a may include a first iodine removal vessel 50a and a second iodine removal vessel 50b. The product stream 44 may be cooled in the first iodine removal vessel 50a to a temperature below the boiling point of the iodine to condense or desublimate at least some of the iodine, separating it from the product stream 44. The product stream 44 may be further cooled in the first iodine removal vessel 50a to a temperature below the melting point of the iodine to separate even more iodine from the product stream 44, depositing at least some of the iodine within the first iodine removal vessel 50a as a solid and producing a reduced iodine product stream 52. The reduced iodine product stream 52 may be provided to the second iodine removal vessel 50b and cooled to separate at least some more of the iodine from the reduced iodine product stream 52 to produce a further crude hydrogen iodide product stream 54.

Although the first iodine removal train 48a consists of two iodine removal vessels operating in a series configuration, it is understood that the first iodine removal train 48a may include two or more iodine removal vessels operating in a parallel configuration, more than two iodine removal vessels operating in a series configuration, or any combination thereof. It is also understood that the first iodine removal train 48a may consist of a single iodine removal vessel. It is further understood that any of the iodine removal vessels may include, or be in the form of, heat exchangers. It is also understood that consecutive vessels may be combined into a single vessel having multiple cooling stages.

The iodine collected in the first iodine removal vessel 50a may form a first iodine recycle stream 56a. Similarly, the iodine collected in the second iodine removal vessel 50b may form a second iodine recycle stream 56b. Each of the first iodine recycle stream 56a and the second iodine recycle stream 56b may be provided continuously or intermittently to the iodine liquefier 20, as shown, and/or to the iodine vaporizer 24.

In order to provide continuous operation while collecting the iodine in solid form, the upstream valve 46 may be configured to selectively direct the product stream 44 to a second iodine removal train 48b. The second iodine removal train 48b may be substantially similar to the first iodine removal train 48a, as described above. Once either the first iodine removal vessel 50a or the second iodine removal vessel 50b of the first iodine removal train 48a accumulates enough solid iodine that it is beneficial to remove the solid iodine, the upstream valve 46 may be selected to direct the product stream 44 from the first iodine removal train 48a to the second iodine removal train 48b. At about the same time, a downstream valve 58 configured to selectively direct the crude hydrogen iodide product stream 54 from either of the first iodine removal train 48a or the second iodine removal train 48b may be selected to direct the crude hydrogen iodide product stream 54 from the second iodine removal train 48b so that the process of removing the iodine from the product stream 44 to produce the crude hydrogen iodide product stream 54 may continue uninterrupted. Once the product stream 44 is no longer directed to the first iodine removal train 48a, the first iodine removal vessel 50a and the second iodine removal vessel 50b of the first iodine removal train 48a may be heated to above the melting point of the iodine, liquefying the solid iodine so that it may flow through the first iodine recycle stream 56a and the second iodine recycle stream 56b of the first iodine removal train 48a to the iodine liquefier 20.

As the process continues and either of the first iodine removal vessel 50a or the second iodine removal vessel 50b of the second iodine removal train 48b accumulates enough solid iodine that it is beneficial to remove the solid iodine, the upstream valve 46 may be selected to direct the product stream 44 from the second iodine removal train 48b back to the first iodine removal train 48a, and the downstream valve 58 may be selected to direct the crude hydrogen iodide product stream 54 from the first iodine removal train 48a so that the process of removing the iodine from the product stream 44 to produce the crude hydrogen iodide product stream 54 may continue uninterrupted. Once the product stream 44 is no longer directed to the second iodine removal train 48b, the first iodine removal vessel 50a and the second iodine removal vessel 50b of the second iodine removal train 48b may be heated to above the melting point of the iodine, liquefying the solid iodine so that it may flow through the first iodine recycle stream 56a and the second iodine recycle stream 56b of the second iodine removal train 48b to the iodine liquefier 20. By continuing to switch between the first iodine removal train 48a and the second iodine removal train 48b, the unreacted iodine in the product stream 44 may be efficiently and continuously removed and recycled.

As described above, the liquid iodine may flow through the first iodine recycle streams 56a and the second iodine recycle streams 56b of the first iodine removal train 48a and the second iodine removal train 48b to the iodine liquefier 20. Alternatively, the liquid iodine may flow through the first iodine recycle streams 56a and the second iodine recycle streams 56b of the first iodine removal train 48a and the second iodine removal train 48b to the iodine vaporizer 24, bypassing the iodine liquefier 20 and the liquid flow controller 26.

The crude HI product stream 54 is provided to a heavies distillation column 60. The heavies distillation column 60 may be configured for the separation of higher boiling point substances, such as hydrogen iodide and residual unreacted iodine, from lower boiling point substances, such as the unreacted hydrogen. A bottom product stream 62 including the hydrogen iodide and residual unreacted iodine from the heavies distillation column 60 may be provided to an iodine recycle column 64. The iodine recycle column 64 may be configured for the separation of the residual unreacted iodine from the hydrogen iodide. A bottoms product stream 66 of the iodine recycle column 64 including the unreacted iodine may be recycled back to the iodine liquefier 20. Alternatively, the bottoms product stream 66 of the iodine recycle column 64 including the unreacted iodine may be recycled back to the iodine vaporizer 24. An overhead product stream 68 of the iodine recycle column 64 including the hydrogen iodide may be provided to a product distillation column 70. An overhead product stream 72 including the hydrogen and residual hydrogen iodide from the heavies distillation column 60 may also be provided to the product distillation column 70. The product distillation column 70 may be configured to separate the unreacted hydrogen from the hydrogen iodide. An overhead product stream 74 of the product column 70 including the unreacted hydrogen and residual hydrogen iodide may be recycled back to the reactor 40. The resulting purified hydrogen iodide product may be collected from a bottom stream 76 of the product column 70.

If desired, more or fewer columns may be used for the separation of hydrogen (H₂) and iodine (I₂) from hydrogen iodide (HI). The columns may be single stage separation unit operations; dual stage separation unit operations, such as vaporizers or reboilers, and/or condensers; or combinations thereof.

The purified HI may comprise greater than 99.5 wt. % HI, less than 3000 ppm iodine species, less than 300 ppm non-volatile residue (NVR), less than 100 ppm hydrogen gas, and trace amounts of organics and moisture. The concentration of HI is determined by titration or ¹H NMR. The total concentration of iodine species is determined by titration using thiosulphate and is a cumulative representation of the attendant concentration of elemental iodine and hydrogen triiodide (HI₃). The NVR may include iodine, diiodopropane, tertbutyl iodide, iodopropane, iodopropene, or other iodo-hydrocarbons.

Iodine-containing species (ICS) may be removed or separated from the stream comprising HI. Multiple different units and configurations thereof may be used to remove iodine from HI, as will be described herein.

As an example, a feedstock or reactant stream (comprising reagents, e.g. hydrogen and iodine, as well as any other carrier fluids and or byproducts or impurities) may be fed to a reactor. The reactor may be generally configured to convert hydrogen and iodine into HI over a catalyst. Before being fed into the reactor, the feedstock may also be mixed with an optional recycle stream, which may comprise unreacted reagents from the reactor effluent stream, such as iodine and/or hydrogen, as well as hydrogen iodide.

The reactor effluent leaves the reactor and then enters ICS removal system. At least a portion of ICS within the reactor effluent stream may be removed within the ICS removal system, and an HI product stream, in addition to an optional waste stream and an optional recycle stream, leaves ICS removal system. The HI product stream may then be sent to another reactor, column, or other process unit to be further reacted or purified. The ICS removal system comprises at least one separation unit, such as an adsorption column, a quenching unit, a distillation column, a condenser, or other separation units and combinations thereof.

In one example, an adsorbent may be used in the removal of an iodine-containing species from hydrogen iodide. The use of such an adsorbent may provide for the efficient removal of an iodine-containing species from hydrogen iodide.

In this method, a stream containing hydrogen iodide (HI) may be passed through the at least one column charged with adsorbent materials after exiting a reactor, such as the reactor configured to produce HI. The HI may be in liquid form, vapor form, or any combination of the two. Preferably, the HI is in liquid form. The adsorption column is operated at a temperature as low as about −50° C., about −40° C., about −30° C., about −20° C., about −10° C., about 0° C., about 10° C., about 20° C., about 30° C. or about 40° C., or as high as about 50° C., about 60° C., about 70° C., about 80° C., about 90° C., about 100° C., about 110° C. or about 120° C. or within any range defined between any two of the foregoing values, such as about −50° C. to about 120° C., as about −30° C. to about 110° C. as about 0° C. to about 100° C., about 10° C. to about 90° C., about 20° C. to about 80° C., about 30° C. to about 70° C., about 40° C. to about 60° C., about 50° C. to about 70° C., about 40° C. to about 50° C., about 60° C. to about 90° C., about 0° C. to about 60° C. or about 20° C. to about 40° C., for example. Preferably, the adsorption column is operated at a temperature of about 0° C. to about 60° C. More preferably, the adsorption column is operated at a temperature of about 20° C. to about 50° C.

The adsorption column may be operated at a pressure slightly above the pressure of the next unit in the process, or at a pressure of as low as about −10 psig, 0 psig, about 5 psig, about 20 psig, about 50 psig, about 70 psig or about 100 psig, or as high as about 150 psig, about 200 psig, about 250 psig, about 300 psig, about 400 psig, about 500 psig, or about 600 psig or within any range defined between any two of the foregoing values, such as about −10 psig to about 600 psig, as about 0 psig to about 500 psig, as about 0 psig to about 400 psig about 5 psig to about 250 psig, about 20 psig to about 200 psig, about 50 psig to about 150 psig, about 5 psig to about 100 psig, about 20 psig to about 70 psig, or about 150 psig to about 250 psig, for example. Preferably, the adsorption column is operated at a pressure of about 5 psig to about 250 psig. More preferably, the adsorption column is operated at a pressure of about 10 psig to about 200 psig.

Non-limiting examples of suitable adsorption materials include silicalite (Al-free ZSM-5), modified silicalites, and aluminosilicate molecular sieves. ZSM-5, Zeolite Socony Mobil-5 (framework type MFI from ZSM-5 (five)), is an aluminosilicate zeolite belonging to the pentasil family of zeolites. Non-limiting examples of modified silicalites include transition metal modified silicalites, alkali metal modified silicalites, alkaline earth metal modified silicalites, rare-earth metal modified silicalite, metal oxide modified silicalites, and metal halide modified silicalites.

Silicalite is one of several forms (polymorphs) of silicon dioxide. It is a white solid. It consists of tetrahedral silicon centers and two-coordinate oxides. It may be prepared by hydrothermal reaction using tetrapropylammonium hydroxide followed by calcining to remove residual ammonium salts. The compound is notable in being 33% porous. The product stream may be in contact with the adsorbent for a contact time as short as about 0.1 second, about 2 seconds, about 4 seconds, about 6 seconds, about 8 seconds, about 10 seconds, about 15 seconds, about 20 seconds, about 25 seconds, or about 30 seconds, or as long as about 40 seconds, about 50 seconds, about 60 seconds, about 70 seconds, about 80 seconds, about 100 seconds, about 120 seconds, about 1,800 seconds, about 3,600 seconds, about 1 hour, about 5 hours, about 10 hours, about 24 hours, about 48 hours, about 72 hours, about 144 hours, or about 168 hours, or about 240 hours. The product stream may be in contact with the adsorbent for a contact time of hours or even days, the residence time being limited only by economic considerations. For example, the product stream may be in contact with the adsorbent for a contact time within any range defined between any two of the foregoing values, such as about 0.1 seconds to about 240 hours, about 0.1 seconds to about 3,600 seconds, about 2 seconds to about 120 seconds, about 4 second to about 100 seconds, about 6 seconds to about 80 seconds, about 8 seconds to about 70 seconds, about 10 seconds to about 60 seconds, about 15 seconds to about 50 seconds, about 20 seconds to about 40 seconds, about 20 seconds to about 30 seconds, about 10 seconds to about 20 seconds, or about 100 seconds to about 120 seconds.

Additionally, adsorption could also be combined with distillation or other separation units or systems as a final treatment step to make high purity HI.

In this method, an iodine containing species may be removed from a mixture of iodine containing species and hydrogen iodide through adsorption. The mixture may comprise purchased HI, HI from production reactor effluent, and/or from purified HI obtained from HI production. The mixture may pass into an iodine removal train comprising two adsorption columns positioned in series. The iodine removal train may be configured to at least partially remove ICS from the HI product. Once a portion of ICS have been removed from the mixture, the HI product stream may leave the ICS removal train and may be fed to a further reactor, column, or other unit.

Although the iodine removal train described above consists of two iodine removal vessels operating in a series configuration, it is understood that the iodine removal train may include two or more iodine removal vessels operation in a parallel configuration, more than two iodine removal vessels operating in a series configuration, and any combination thereof. It is also understood that the iodine removal train may consist of a single iodine removal vessel.

The iodine collected in the first adsorption column may be removed to form a first iodine recycle stream. Similarly, the iodine collected in the second adsorption column may be removed to form a second iodine recycle stream. Each of the first iodine recycle stream and the second iodine recycle stream may be provided to an iodine liquefier to recycle for HI production.

Alternatively, the ICS removal system may comprise a quenching unit, where the reactor effluent comprising HI, unreacted iodine, unreacted hydrogen, and potential by-products, small amounts of water, etc. may be quenched with HI liquid. The HI liquid may capture the incoming ICS. Depending on the operating conditions, the captured ICS may be completely miscible with HI liquid forming one liquid phase, or the ICS may be a solid, forming a slurry with the HI liquid. The HI containing ICS may be partially evaporated to remove some of the HI, and the remaining HI and ICS may be recycled to the reactor. The quenching unit may contact the incoming reactor effluent (typically a vapor) with quenching liquid HI through any liquid-gas contacting device, such as through a sparger or multiple spargers submerged in HI liquid, distillation trays, shower curtain trays, slant trays, shed trays, random packing, structured packing, liquid spraying devices or nozzles, or any other suitable system/device or combinations thereof.

As an example, the quenching system may comprise a quencher, which may be generally described as a liquid-gas contactor. The system may also comprise an evaporator, a first distillation column equipped with a condenser and a reboiler, and a second distillation column.

The reactor effluent may be quenched with HI liquid within the quencher. The HI liquid or the contacting liquid can be the HI produced from the process and subsequently be replenished by HI produced in step 1, or may be replenished in part or in entirety by increasing the HI reflux. The HI replenishing liquid may also be supplied from the distillation column. Furthermore, HI may be added to the reactor effluent to assist diluting the iodine concentration in the vapor stream feeding to the quenching HI liquid, which may lessen the localized solid formation when it first contacts with the HI liquid. The quenching HI liquid may be introduced downstream of the quench, which will eventually flow back into the quencher.

The energy exchange between the hot reactor effluent vapor and the HI liquid may result in evaporating off a portion of the HI liquid. This evaporated HI, along with potentially a very small amount of I₂ from the quenching pool mixture may be sent to a multi-stage distillation column having sufficient rectifying stages and an overhead condenser to remove the HI from the residual iodine. Since the partial pressure of I₂ is small relative to the HI coming off from the quenching pool mixture, the vapor content or the quantity of the I₂ may be managed within the distillation column without major solid deposit as I₂ exhibits some solubility in the HI liquid. This pre-conditioning step may eliminate any messy and solid deposit occurrence afterward, allowing a reliable downstream processing such as compressor and distillation operation. A portion of the evaporated HI vapor may be condensed by the overhead condenser and refluxed and sent to the distillation column to dissolve this residual I₂.

The distillation column bottoms, containing residual iodine and HI liquid, may be sent back to the quencher to replenish the HI liquid being evaporated. The quencher may be integrated directly with the distillation column, which may reduce the required amount of piping in the system.

The collected ICS in the quench pool is then sent to an evaporator to evaporate a majority of the HI, leaving iodine and some HI to be collected or recycled to the reactor for HI production. The evaporated HI containing some ICS from this evaporator is sent back to the distillation column and/or to the quenching pool to again separate out the HI and ICS in the same manner as described earlier.

As an alternative to the method of iodine collection described above, after the HI liquid pool has collected a sufficient amount of ICS, the hot reactor effluent vapor may be switched into another quencher to continue the ICS collection mode in the other quencher. The HI liquid pool containing ICS may then then evaporated to remove a majority of the HI as described above, and sent back to the distillation and/or to the new quench pool to again separate out the HI and ICS in the same manner as described earlier. The remaining I₂ and some HI may be recycled to the reactor. The two set of HI liquid pools/quenchers may be configured and sequenced in alternating ICS collection mode and ICS recycle mode. The advantage of having alternating, multiple, or redundant ICS collection equipment may provide the ability to readily isolate the equipment for service in the event of pluggage caused by the Iodine or other equipment failure or maintenance. This configuration may also eliminate or reduce need for a solid handling pump to boost the operating pressure necessary during the ICS recycle mode.

The HI along with the H₂, having been purified and separated from the ICS in the distillation column, may leave in the distillation overhead. It may then be processed further to separate the HI from the H₂ stream to derive a recycle stream comprising H₂ for recycling to the reactor. A portion of the liquefied HI may be used to replenish the quenching operation as described earlier. The remaining liquefied HI portion is the purified HI product, which may then be sent to another unit for further use or processing.

### 2. Formation of Trifluoroacetyl Iodide (TFAI)

As disclosed herein, in a second reaction step of the integrated process, TFAC (trifluoroacetyl chloride) is reacted with HI (hydrogen iodide) to form an intermediate product stream comprising TFAI (trifluoroacetyl iodide) and HCl (hydrogen chloride) according to Eq. 2:

TFAC+HI→TFAI+HCl

The process may be conducted in gas phase, liquid phase, or gas/liquid phase. The process comprises providing a reactant stream comprising hydrogen iodide and at least one trifluoroacetyl halide selected from trifluoroacetyl chloride (TFAC), trifluoroacetyl fluoride (TFAF), trifluoroacetyl bromide (TFAB), and combinations thereof, to produce an intermediate product stream comprising the trifluoroacetyl iodide (TFAI).

The process may be conducted in a reactor, such as a heated tube reactor comprising a tube made of a metal such as carbon steel, stainless steel, nickel, and/or a nickel alloy, such as a nickel-chromium alloy, a nickel-molybdenum alloy, a nickel-chromium-molybdenum alloy, or a nickel-copper alloy. Alternatively, the reactor may be constructed of a metal lined with glass or polymers such as polytetrafluoroethylene (PTFE), perfluoroalkoxy alkanes (PFA), fluorinated ethylene propylene (FEP) and other fluoropolymers. The reactor may be heated, or the feed materials may be preheated before entering the reactor. The reactor may be any type of packed bed reactor.

The hydrogen iodide and the trifluoroacetyl chloride in the reactant stream may optionally react in the presence of a catalyst contained within the reactor. When the catalyst is used, it may be selected from the group comprising activated carbon, meso carbon, stainless steel, nickel, nickel-chromium alloy, nickel-chromium-molybdenum alloy, nickel-copper alloy, copper, alumina, platinum, palladium, or carbides, such as metal carbides, such as iron carbide, molybdenum carbide and nickel carbide, and non-metal carbides, such as silicon carbide, or combinations thereof. The catalyst may be in the form of a mesh, pellet, or sphere, contained within the reactor.

It is desirable to produce TFAI low in impurities such that factors such as the yield, operability and cost (among others) of the process is optimized. Therefore, composition of the reactor feed material comprising HI and TFAC is important. The feed to the reactor includes fresh HI, fresh TFAC, which may be treated to remove sulfur dioxide (SO₂) if required and as described below, as well as recycle comprising unreacted HI and TFAC. According to one embodiment, the composition comprises a plurality of components wherein the sum of TFAC and HI comprises at least 99 wt. %, sulfur dioxide (SO₂) may be present in an amount of not more than 250 ppm, the sum of iodine and HI₃ may be no more than 2000 ppm, iodohydrocarbons may be present in an amount of not more than 500 ppm, hydrogen may be present in an amount of not more than 500 ppm, and CF₃I may be present in an amount of not more than 5000 ppm. Iodohydrocarbons include iodomethane, iodoethane, iodopropane, iodobutane, tert-butyl iodide, diiodopropane, and others.

The reaction temperatures may be as low as about 0° C. or higher, about 25° C. or higher, about 35° C. or higher, about 40° C. or higher, about 50° C. or higher, or about 60° C. or lower, about 90° C. or lower, about 120° C. or lower, about 150° C. or lower, or about 200° C. or lower, or about 250° C. or lower, or any value encompassed by these endpoints.

The reaction pressure may be about 0 psig or higher, about 25 psig or higher, about 50 psig or higher, about 50 psig or higher, about 100 psig or higher, about 150 psig or higher, about 200 psig or higher, about 250 psig or higher, about 300 psig or lower, about 350 psig or lower, about 400 psig or lower, about 450 psig or lower, about 500 psig or lower, or any value encompassed by these endpoints.

The reactant stream may be in contact with the catalyst for a contact time of about 0.1 seconds or longer, about 0.5 seconds or longer, about 1 second or longer, about 2 seconds or longer, about 3 seconds or longer, about 5 seconds or longer, about 8 seconds or longer, about 10 seconds or longer, about 12 seconds or longer, or about 15 or longer, about 18 seconds or longer, about 20 seconds or shorter, about 25 seconds or shorter, about 30 seconds or shorter, about 35 seconds or shorter, about 40 seconds or shorter, about 50 seconds or shorter, about 60 seconds or shorter, about 80 seconds or shorter, or about 300 seconds shorter, or about 1800 seconds shorter, or any value encompassed by these endpoints.

In an example of this process, fresh HI and TFAC are combined with a recycle mixture comprising HI and TFAC recovered from processing downstream, for example a distillation train. The combined TFAC/HI mole ratio is provided with an excess of TFAC to give high conversion of the more expensive HI, although equimolar amounts or an excess of HI may also be used.

In one embodiment, the TFAC/HI mole ratio may be as low as about 1:10, about 1:5, about 1:2, about 1:1, about 2:1, about 3:1, about 4:1, or as high as about 5:1, about 6:1, about 7:1, about 8:1, about 9:1, or about 10:1, or within any range defined between any two of the foregoing values. Preferably, the TFAC/HI ratio is from 1:2 to 2:1. More preferably, the TFAC/HI ratio is from 1:1 to 2:1.

In another embodiment, the TFAC/HI mole ratio may be about 1:1 or less, about 0.9:1 or less, about 0.8:1 or less, about 0.7:1 or less, or about 0.6:1 or less, or about 0.1:1 or less, or about 0.05:1 or less, or about 0.02:1 or less.

The mixture may be vaporized and superheated to perform the reaction.

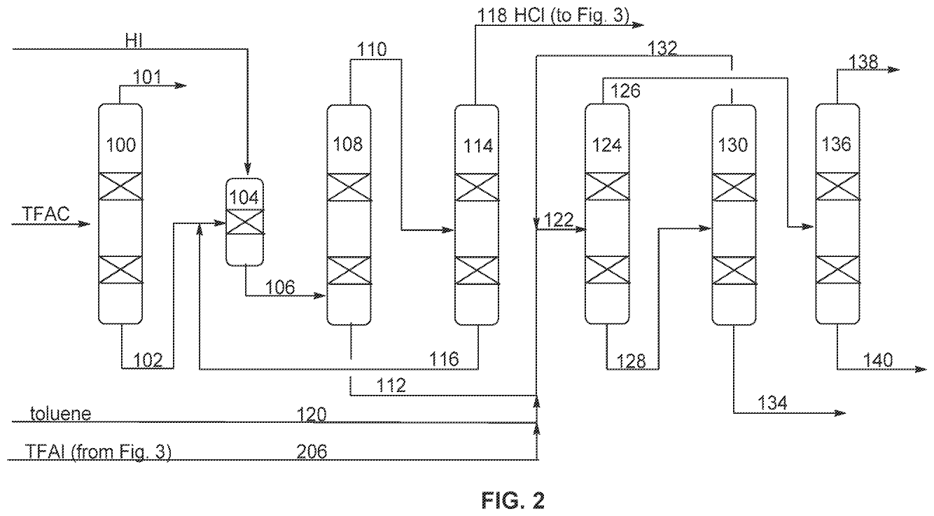

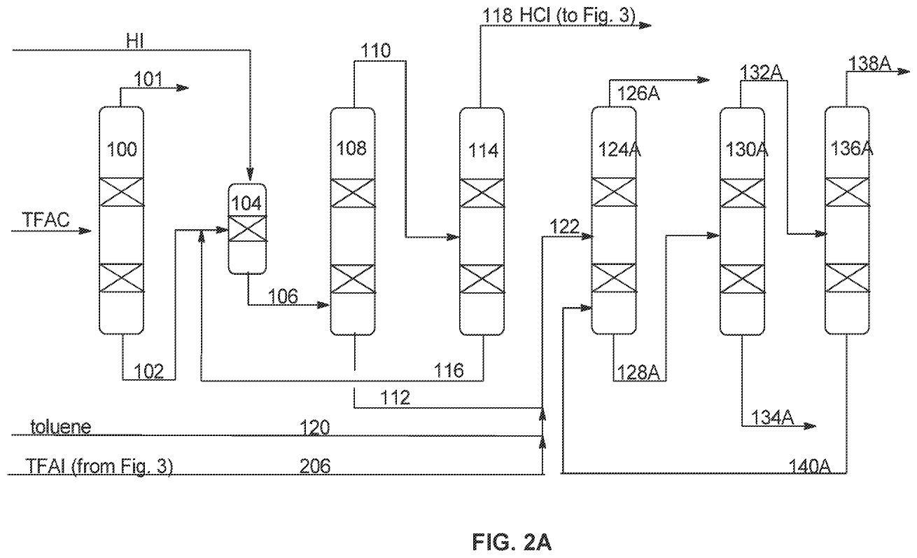

A schematic of an example for a second reaction step, including some of the purification options further discussed below, is shown in FIG. 2. A first feed stream comprising TFAC is conveyed to a first column 100 to remove sulfur dioxide (SO₂), providing a bottoms product stream 102 comprising TFAC with a reduced concentration of sulfur dioxide and an overhead stream 101 comprising an azeotrope or near-azeotrope of TFAC and SO₂. The product stream 102 may then be combined with a recycle stream 116 comprising unreacted TFAC and unreacted HI and conveyed to a reactor 104. A second feed stream comprising HI from Step 1 of the process, described above, may also be fed to reactor 104 or combined with streams 102 and 116 before being fed to reactor 104 to provide a product stream 106 comprising crude TFAI, hydrogen chloride (HCl), iodine, trifluoroacetic acid (TFA), HI, and TFAC. Product stream 106 may be conveyed to a second column 108 to provide an overhead product stream 110 comprising HCl, TFAC, and HI and a bottoms product stream 112 comprising TFAI, iodine, HI₃ and TFA. The overhead product stream 110 may be conveyed to a third column 114 to provide a bottoms product stream 116 which may be recycled back to reactor 104, and an overhead product stream 118 comprising HCl. The overhead product stream 118 may then be conveyed to Step 3 of the integrated process or recovered.

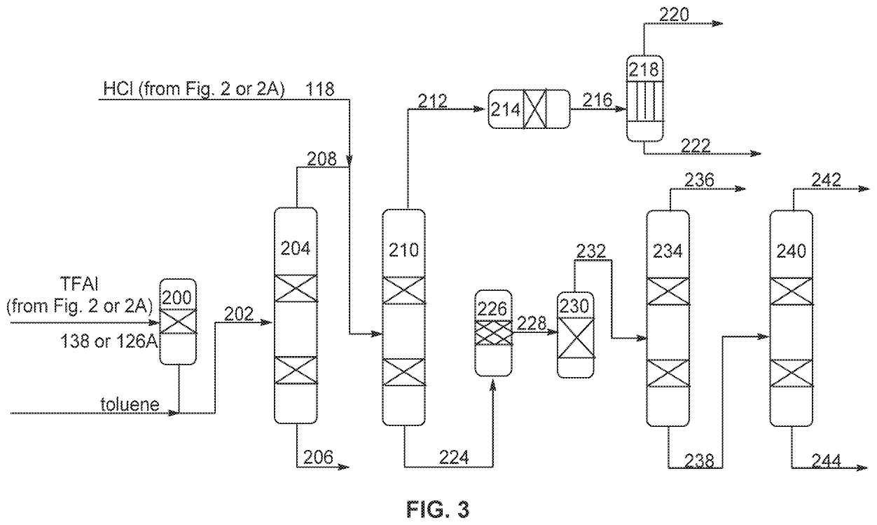

The bottoms product 112 from the second column 108 may be combined with recycled TFAI from Step 3 of the process (stream 206 from FIG. 3) and with a stream of solvent 120, such as toluene, to prevent iodine in stream 112 from solidifying in a combined stream 122 as well as to act as a solvent to selectively absorb iodine from the mixture comprising TFAI in a fourth column. The stream 122 may be conveyed to a fourth column 124 to provide an overhead product stream 126 comprising TFAI and TFA and a bottoms product stream 128 comprising toluene and iodine. The bottoms product stream 128 may be conveyed to a fifth column 130 to provide an overhead product stream 132 comprising toluene, which may be recycled to the fourth column 124. Stream 132 may be combined with stream 122 before being fed to the fourth column or fed separately into the fourth column. The bottoms product stream 134 from the fifth column 130, comprising iodine, may be collected or recycled back to Step 1 of the integrated process. The overhead product stream 126 from the fourth column may be conveyed to a sixth column 136, to provide an overhead product stream 138, comprising TFAI, which may be passed to Step 3 of the integrated process. The bottoms product stream 140, comprising TFA and other high-boiling impurities may be collected for other use or disposal.

Alternatively, referring to FIG. 2A, the bottoms product 112 from the second column 108 may be combined with recycled TFAI from Step 3 of the process (stream 206) and with a stream of solvent 120, such as toluene, to prevent iodine in stream 112 from solidifying in a combined stream 122 as well as to act as a solvent to selectively absorb iodine from the mixture comprising TFAI. The stream 122 may be conveyed to a fourth column 124A to provide an overhead product stream 126A comprising TFAI which may be passed to Step 3 of the integrated process and a bottoms product stream 128A comprising TFA, iodine, and toluene. The bottoms product stream 128A may be conveyed to a fifth column 130A to provide an overhead product stream 132A comprising TFA and toluene, which may be conveyed to a sixth column 136A. The bottoms product stream 134A from the fifth column 130, comprising iodine, may be collected or recycled back to Step 1 of the integrated process. The bottoms product 140A of the sixth column 136 comprising toluene may be recycled to fourth column 124. The overhead product stream 138A, comprising TFA and other impurities may be collected for other use or disposal.

### 3. Purification of Trifluoroacetyl Chloride (TFAC)

A commercial supply of TFAC may contain a sulfur dioxide impurity which forms a minimum boiling azeotrope with TFAC. It is desirable to remove the sulfur dioxide before feeding into the reaction train.

One method by which TFAC may be purified includes the formation of an azeotrope or azeotrope-like composition with sulfur dioxide. This azeotrope or azeotrope-like composition may be removed from the bulk TFAC via distillation. Another method by which sulfur dioxide may be removed from TFAC includes contacting a mixture of TFAC and sulfur dioxide with a solid adsorbent or a mixture of two or more solid adsorbents to remove sulfur dioxide from the mixture of TFAC and sulfur dioxide. Yet another method by which TFAC may be purified includes a combination of these methods. Contacting the mixture of TFAC and sulfur dioxide with a solid adsorbent may precede or follow distillation. Multiple adsorbent steps may be used, with or without distillation.

It has been found that TFAC forms homogeneous, minimum boiling azeotrope and azeotrope-like compositions or mixtures with sulfur dioxide, and the present disclosure provides homogeneous azeotrope or azeotrope-like compositions comprising TFAC and sulfur dioxide. The azeotrope or azeotrope-like compositions may consist essentially of TFAC and sulfur dioxide or the azeotrope or azeotrope-like compositions may consist of TFAC and sulfur dioxide.

An “azeotrope” composition is a unique combination of two or more components. An azeotrope composition can be characterized in various ways. For example, at a given pressure, an azeotrope composition boils at a constant characteristic temperature which is either greater than the higher boiling point component (maximum boiling azeotrope) or less than the lower boiling point component (minimum boiling azeotrope). At this characteristic temperature the same composition will exist in both the vapor and liquid phases. The azeotrope composition does not fractionate upon boiling or evaporation. Therefore, the components of the azeotrope composition cannot be separated during a phase change.

Alternatively, an azeotrope composition may be characterized as a composition which boils at a characteristic vapor pressure at a given temperature. The vapor pressure may be lower than the lower vapor pressure component (pressure minimum azeotrope) or the vapor pressure may be higher than the higher vapor pressure component (pressure maximum azeotrope). A pressure minimum azeotrope may be referred to as a maximum boiling azeotrope, or vice versa, and a pressure maximum azeotrope may be referred to as a minimum boiling azeotrope, or vice versa.

The behavior of an azeotrope composition is in contrast with that of a non-azeotrope composition in which during boiling or evaporation, the liquid composition changes to a substantial degree.

One of ordinary skill in the art would understand however that at different pressures, both the composition and the boiling point of the azeotrope composition will vary to some extent. Therefore, depending on the temperature and/or pressure, an azeotrope composition can have a variable composition. The skilled person would therefore understand that composition ranges, rather than fixed compositions, can be used to define azeotrope compositions. In addition, an azeotrope may be defined in terms of exact weight percentages of each component of the compositions characterized by a fixed boiling point at a specified pressure.

An “azeotrope-like” composition is a composition of two or more components which behaves substantially as an azeotrope composition. Thus, for the purposes of this disclosure, an azeotrope-like composition is a combination of two or more different components which, when in liquid form under given pressure, will boil at a substantially constant temperature, and which will provide a vapor composition substantially identical to the liquid composition undergoing boiling.

As used herein, the term “consisting essentially of”, with respect to the components of an azeotrope or azeotrope-like composition or mixture, means the composition contains the indicated components in an azeotrope or azeotrope-like ratio, and may contain additional components provided that the additional components do not form new azeotrope or azeotrope-like systems. For example, azeotrope mixtures consisting essentially of two compounds are those that form binary azeotropes, which optionally may include one or more additional components, provided that the additional components do not render the mixture non-azeotropic and do not form an azeotrope with either or both of the compounds (e.g., do not form a ternary or higher azeotrope).

The azeotrope or azeotrope-like composition having a boiling point of about 10.0° C.±3° C. at a pressure of about 45 psia±0.3 psia may comprise, consist essentially of, or consist of, from about 25 wt. % to about 99 wt. % TFAC and from about 1 wt. % to about 75 wt. % sulfur dioxide, from about 48 wt. % to about 90 wt. % TFAC and from about 10 wt. % to about 52 wt. % sulfur dioxide, or from about 68 wt. % to about 78 wt. % TFAC and from about 22 wt. % to about 32 wt. % sulfur dioxide.

Alternatively, the azeotrope or azeotrope-like composition having a boiling point of about 10.0° C.±3° C. at a pressure of about 45 psia±0.3 psia may comprise, consist essentially of, or consist of, from about 24.5 wt. % to about 94.9 wt. % TFAC and from about 5.1 wt. % to about 75.5 wt. % sulfur dioxide, or from about 47.9 wt. % to about 89.7 wt. % TFAC and from about 10.3 wt. % to about 52.1 wt. % sulfur dioxide.

The present disclosure also provides a composition comprising the azeotrope or azeotrope-like composition. For example, there is provided a composition comprising at low as 1 ppm of the azeotrope or azeotrope-like compositions, 10 ppm of the azeotrope or azeotrope-like compositions, 25 ppm of the azeotrope or azeotrope-like compositions, or as high as 50 ppm of the azeotrope or azeotrope-like compositions, 100 ppm of the azeotrope or azeotrope-like compositions, 1000 ppm of the azeotrope or azeotrope-like compositions, 1 wt. % of the azeotrope or azeotrope-like compositions, 5 wt. % or more of the azeotrope or azeotrope-like compositions.

Following the separation of the azeotrope or azeotrope-like composition from another composition, the azeotropic composition may include at least 10 wt. % of the azeotrope or azeotrope-like compositions, or at least about 20 wt. % of the azeotrope or azeotrope-like compositions, or at least about 50 wt. % of the azeotrope or azeotrope-like compositions, or at least about 70 wt. % of the azeotrope or azeotrope-like compositions, or at least about 90 wt. % of the azeotrope or azeotrope-like compositions.

The azeotrope or azeotrope-like composition comprising, consisting essentially of, or consisting of effective amounts of TFAC and sulfur dioxide disclosed herein may be used for separating impurities, including sulfur dioxide, from TFAC.

In particular, an azeotrope or azeotrope-like composition comprising, consisting essentially of, or consisting of effective amounts of TFAC and sulfur dioxide may be formed from a composition including one or both of TFAC and sulfur dioxide, optionally together with one or more other chemical compounds other than TFAC and sulfur dioxide, such as other impurities. Following the formation of the azeotrope or azeotrope-like composition, the azeotrope or azeotrope-like composition may be separated from the other chemical compounds by a suitable method, such as by distillation or fractionation.

The present disclosure provides a method of separating sulfur dioxide as an impurity from a crude composition of TFAC which includes sulfur dioxide as an impurity, together with any additional impurities, if present. The sulfur dioxide may be present in the crude composition of TFAC in an amount of about 5 ppm or greater, about 50 ppm or greater, about 100 ppm or greater, about 500 ppm or greater, about 1000 ppm or greater, about 2000 ppm or greater, about 3000 ppm or greater, or about 5000 ppm or greater.

One method comprises the steps of providing crude TFAC, sulfur dioxide as an impurity, and any other impurities, if present; conveying the crude TFAC to a distillation column; collecting the distillate from the distillation column, the distillate comprising sulfur dioxide, or an azeotrope or azeotrope-like mixture of sulfur dioxide and TFAC; and collecting the bottoms product stream from the distillation column, the bottoms product stream consisting essentially of TFAC.

Another method comprises the steps of providing a crude composition comprising TFAC, sulfur dioxide as an impurity, and any other impurities, if present, and subjecting the crude composition to conditions effective to form an azeotrope or azeotrope-like composition consisting essentially of, or consisting of, effective amounts of TFAC and sulfur dioxide, and separating the azeotrope or azeotrope-like composition from the crude composition by a separation technique such as distillation, or fractionation, for example. Thereafter, the azeotrope or azeotrope-like composition may be subjected to further separation or purification steps to obtain purified TFAC.

A further method to separate TFAC and sulfur dioxide from a feed stream including TFAC and sulfur dioxide may include the formation of an azeotrope or azeotrope-like composition or may not include the formation of an azeotrope or azeotrope-like composition. The method includes an initial step of conveying a feed stream including TFAC and sulfur dioxide to a distillation column to provide both a bottoms product stream and an overhead product stream. The bottoms product stream may be passed through a reboiler, and a portion of it may be recycled back to the column, while another portion of it may be collected as a bottoms product stream. The bottoms product stream consists essentially of TFAC. The overhead product stream may be passed through a condenser, and a portion of it may be refluxed back to the column while the remainder may be collected as an overhead product stream. The overhead product stream comprises an azeotrope or azeotrope-like composition consisting essentially of effective amounts of TFAC and sulfur dioxide. The product stream may further comprise excess TFAC. The column may be operated under various temperature and pressure conditions to achieve the desired separation.

The bottoms product stream may include sulfur dioxide in an amount of about 100 ppm or less, about 50 ppm or less, about 10 ppm or less, or about 1 ppm or less.

In another example, the present disclosure provides a method of separating sulfur dioxide as an impurity from a crude composition of TFAC which includes sulfur dioxide as an impurity, together with at least one additional impurity, comprising the steps of providing a composition of crude TFAC, sulfur dioxide as an impurity, and at least one additional impurity, and contacting the crude composition with a solid adsorbent.

Suitable adsorbents may include molecular sieves, such as 3 Å molecular sieves available from Acros Organics (also available from Honeywell UOP); 4 Å and XH-9 molecular sieves available from Honeywell UOP, 10 Å molecular sieves available from Grace Davison, and carbon molecular sieves, such as MSC-3K 172 carbon molecular sieves available from Osaka Gas Chemicals; activated alumina, such as SAS40 ⅛″ Alumina available from BASF; zeolite ammonium powders, such as CBV5524G CY available from Zeolyst International; and activated charcoal, such as NORIT ROX 0.8 Activated Carbon available from Cabot.

In this method, sulfur dioxide may be removed from TFAC through an adsorption process by contacting the SO₂-containing TFAC feed stream with an adsorbent. This contact may be mediated via a pump to move the feed stream through a packed bed by pressure differential. Once contacted by the adsorbent, the feed stream may be sent forward for further processing or recirculated from the bed back to the holding vessel until the desired purity is achieved.

The recirculation process (or adsorption process) can be operated at temperatures ranging of about −30° C. or higher, about −20° C. or higher, about −10° C. or higher, about 0° C. or higher, about 10° C. or higher, about 20° C. or lower, about 30° C. or lower, about 40° C. or lower, about 50° C. or lower, about 60° C. or lower, about 70° C. or lower, about 80° C. or lower, about 90° C. or lower, or any value encompassed by these endpoints.

The recirculation process (or adsorption process) can be operated at pressures ranging of about 0 psig or higher, about 5 psig or higher, about 10 psig or higher, about 20 psig or higher, about 50 psig or higher, about 100 psig or higher, about 120 psig or lower, about 150 psig or lower, about 200 psig or lower, about 300 psig or lower, about 500 psig or lower, or any value encompassed by these endpoints.

### 4. Purification of Trifluoroacetyl Iodide (TFAI)

The reaction to form trifluoroacetyl iodide (TFAI) from TFAC and HI generally proceeds with a high degree of selectivity for TFAI. The main impurities in the intermediate product stream may be unreacted starting materials (TFAC and HI), and minor impurities may include acidic by-products such as unreacted HI and trifluoroacetic acid (TFA). The major by-product of the reaction may include hydrogen chloride (HCl), along with minor amounts of carbon monoxide (CO) and trifluoroiodomethane (CF₃I). The boiling points of these by-products are lower than that of TFAI; therefore, they may be separated from TFAI by distillation.

The composition of the organic compounds in the intermediate product stream may be measured as by gas chromatography (GC) and gas chromatography-mass spectroscopy (GC-MS) analyses. Graph areas provided by the GC analysis for each of the organic compounds can be combined to provide a GC area percentage (GC area %) of the total organic compounds for each of the organic compounds as a measurement of the relative concentrations of the organic compounds in the intermediate product stream.

The concentration of trifluoroacetyl iodide in the intermediate product stream, in GC area % of total organic compounds, may be as about 10% or greater, about 15% or greater, about 20% or greater, about 25% or greater, about 30% or greater, about 35% or greater, about 40% or greater, about 45% or greater, about 50% or greater, about 55% or greater, about 60% or less, about 65% or less, about 70% or less, about 75% or less, about 80% or less, about 85% or less, about 90% or less, about 95% or less, about 99% or less, or any value encompassed by these endpoints.

The concentration of unreacted trifluoroacetyl chloride in the intermediate product stream, in GC area % of total organic compounds, may be about 1% or greater, about 5% or greater, about 10% or greater, about 15% or greater, about 20% or greater, about 25% or greater, about 30% or greater, about 35% or greater, about 40% or greater, about 45% or greater, about 50% or greater, about 55% or greater, about 60% or less, about 65% or less, about 70% or less, about 75% or less, about 80% or less, about 85% or less, about 90% or less, or any value encompassed by these endpoints.

The concentration of trifluoroiodomethane in the intermediate product stream, in GC area % of total organic compounds, may be about 10% or less, about 8% or less, about 6% or less, about 4% or less, about 3% or less, about 2.5% or less, about 2% or less, about 1.5% or less, about 1% or less, about 0.5% or less, about 0.3% or less, about 0.2% or less, about 0.1% or less, about 0.01% or less, about 0.001% or less, or any value encompassed by these endpoints.

The concentration of all other organic compounds in the intermediate product stream, in GC area % of total organic compounds, may be about 15%, or less about 14% or less, about 13% or less, about 12% or less, about 11% or less, about 10% or less, about 9% or less, about 8% or less, about 7% or less, about 6% or less, about 5% or less, about 4% or less, about 3% or less, about 2% or less, about 1% or less, about 0.5% or less, or about 0.1% or less, or any value encompassed by these endpoints.

The intermediate product stream may be directed to a first distillation column to separate an overhead product stream comprising TFAC, HI, HCl, CF₃I, CO from TFAI, trifluoroacetic acid (TFA), I₂ and other higher boiling point substances in the bottoms product stream of the first distillation column. The overhead product stream of the first distillation column is directed to a second distillation column operating at a higher pressure than the first distillation column. Optionally, the overhead product stream of the first distillation column is directed to a second distillation column via a compressor. The overhead product stream of the second distillation column may include mainly HCl. The bottoms product stream of the second distillation column may include mainly unreacted HI and TFAC, which may optionally be recycled. The bottoms product stream of the first distillation column, including mainly TFAI with minor amounts of trifluoroacetic acid (TFA) and I₂, may be combined with recycled TFAI from Step 3 of the integrated process, shown above.

The overhead product stream of the first distillation column may be at a temperature of about −50° C. or higher, about −40° C. or higher, about −30° C. or higher, about −20° C. or higher, about −10° C. or higher, about 0° C. or higher, about 10° C. or lower, about 20° C. or lower, about 30° C. or lower, about 40° C. or lower, about 50° C. or lower, [or any value encompassed by these endpoints.

The bottoms product stream of the first distillation column may generally be maintained at a temperature below about 150° C., such as about 150° C. or less, about 140° C. or less, about 130° C. or less, about 120° C. or less, about 110° C. or less, or about 100° C. or less, or about 80° C. or less, or about 70° C. or less.

The first distillation column may be operated at a pressure of about 0 psig or higher, about 10 psig or higher, about 25 psig or higher, about 50 psig or higher, about 75 psig or higher, about 100 psig or higher, about 125 psig or higher, about 150 psig or higher, about 175 psig or less, about 200 psig or less, about 225 psig or less, about 250 psig or less, about 275 psig or less, about 300 psig or less, or any value encompassed by these endpoints.

The overhead product stream of the second distillation column may be at a temperature of about −60° C. or higher, about −50° C. or higher, about −40° C. or higher, about −30° C. or lower, about −20° C. or lower, about −10° C. or lower, about −5° C. or lower, about 0° C. or lower, or any value encompassed by these endpoints.

The bottoms product stream of the second distillation column may generally be maintained at a temperature below about 150° C., such as about 150° C. or less, about 140° C. or less, about 130° C. or less, about 120° C. or less, about 110° C. or less, or about 100° C. or less.

The second distillation column may be operated at a pressure of about 20 psig or greater, about 40 psig or greater, about 60 psig or greater, about 80 psig or greater, about 100 psig or greater, about 120 psig or greater, about 140 psig or greater, about 160 psig or greater, about 180 psig or greater, about 200 psig or lower, about 220 psig or lower, about 240 psig or lower, about 260 psig or lower, about 280 psig or lower, about 300 psig or lower, about 320 psig or lower, about 340 psig or lower, about 350 psig or lower, or any value encompassed by these endpoints.

Alternatively, the intermediate product stream may be directed to a first distillation column to separate an overhead product stream comprising mainly HCl from TFAI, HI, TFAC and other higher boiling point substances in the bottoms product stream of the first distillation column. The bottoms product stream of the first distillation column is directed to a second distillation column. The overhead product stream of the second distillation column comprises mainly HI and TFAC, which is optionally recycled. The bottoms product stream of the second distillation column comprises mainly TFAI, along with minor amounts of TFA and I₂, which may be combined with recycled TFAI from Step 3 of the integrated process.

In this alternative process, the overhead product stream of the first distillation column may be at a temperature of about −60° C. or higher, about −55° C. or higher, about −50° C. or higher, about −45° C. or higher, about −40° C. or higher, about −35° C. or higher, about −30° C. or higher, about −25° C. or lower, about −20° C. or lower, about −15° C. or lower, about −10° C. or lower, about −5° C. or lower, about 0° C. or lower, or any value encompassed by these endpoints.

The bottoms product stream of the first distillation column may be at a temperature of about 20° C. or higher, 30° C. or higher, 40° C. or higher, about 60° C. or higher, about 80° C. or higher, about 100° C. or higher, about 125° C. or higher, about 150° C. or lower, about 125° C. or lower, about 100° C. or lower, about 80° C. or lower, about 60° C. or lower, about 40° C. or lower, about 30° C. or lower or any value encompassed by these endpoints.

The first distillation column may be operated at a pressure of about 20 psig or greater, about 40 psig or greater, about 60 psig or greater, about 80 psig or greater, about 100 psig or greater, about 120 psig or greater, about 140 psig or greater, about 160 psig or greater, about 180 psig or greater, about 200 psig or greater, about 220 psig or lower, about 240 psig or lower, about 260 psig or lower, about 280 psig or lower, about 300 psig or lower, about 320 psig or lower, about 340 psig or lower, about 350 psig or lower, or any value encompassed by these endpoints.

The overhead product stream of the second distillation column may be at a temperature of about −30° C. or higher, about −20° C. or higher, about −10° C. or higher, about 0° C. or higher, 10° C. or higher, about 20° C. or higher, about 30° C. or higher, about 40° C. or higher, about 50° C. or higher, about 60° C. or higher, about 70° C. or higher, about 80° C. or lower, about 70° C. or lower, about 60° C. or lower, about 50° C. or lower, about 40° C. or lower, about 30° C. or lower, about 20° C. or lower, about 10° C. or lower, about 0° C., about −10° C. or lower, about −20° C. or lower or any value encompassed by these endpoints.

The bottoms product stream of the second distillation may be at a temperature of about 20° C. or higher, 30° C. or higher, 40° C. or higher, about 60° C. or higher, about 80° C. or higher, about 100° C. or higher, about 125° C. or higher, about 150° C. or lower, about 125° C. or lower, about 100° C. or lower, about 80° C. or lower, about 60° C. or lower, about 40° C. or lower, about 30° C. or lower or any value encompassed by these endpoints.

The second distillation column may be operated a pressure of about 0 psig or greater, about 10 psig or greater about 20 psig or greater, about 40 psig or greater, about 60 psig or greater, about 80 psig or greater, about 100 psig or greater, about 120 psig or greater, about 140 psig or greater, about 160 psig or greater, about 180 psig or greater, about 200 psig or greater, about 225 psig or greater about 250 psig or lower, about 225 psig or lower, about 200 psig or lower, about 180 psig or lower, about 160 psig or lower, about 140 psig or lower, about 120 psig or lower, about 100 psig or lower, about 80 psig or lower, about 60 psig or lower, about 40 psig or lower, about 20 psig, about 10 psig or lower, or any value encompassed by these endpoints.

It is understood that these operating conditions are exemplary only. The person of skill in the art will readily understand that, if for example, the operating pressure is changed, the operating temperatures will also change for the same compositions.

The concentration of the trifluoroacetyl iodide in the purified intermediate product stream may be greater than about 97%. Preferably, the concentration of the trifluoroacetyl iodide in the purified intermediate product stream may be greater than about 99%. More preferably, the concentration of the trifluoroacetyl iodide in the purified intermediate product stream may be greater than about 99.5%. Most preferably, the concentration of the trifluoroacetyl iodide in the purified intermediate product stream may be greater than about 99.9%.

The purified intermediate product stream may be stored, or may be provided to a second reactor for conversion into trifluoroiodomethane. The purified intermediate product stream comprising the trifluoroacetyl iodide may be provided directly to the second reactor. Alternatively, or additionally, the purified intermediate product stream may pass through a preheater to heat the purified intermediate product stream before the purified intermediate product stream is provided to the second reactor.

During the second step of the reaction (the formation of TFAI from TFAC and HI), it is possible that some CF₃I may form, particularly at elevated temperatures. However, the decomposition of TFAI to CF₃I and carbon monoxide (CO) does not go to completion, and may therefore form a mixture comprising, among others, TFAI, CF₃I, CO, TFAC, and HCl. This may lead to additional capital expenditure in purification equipment, as TFAC and CF₃I may form an azeotrope or azeotrope-like composition or may be otherwise difficult to separate by distillation due to very close boiling points. Thus, any CF₃I formed in Step 2 may represent a yield loss or additional equipment and expenditure. Therefore, minimizing or possibly eliminating the formation of CF₃I during this step may represent a significant improvement to the process.

To reduce the formation of CF₃I in this step, it has been found that bulk temperatures above 150° C. should be avoided. The process may be optimized by adjusting column temperatures such that they are operated at less than this temperature whenever a significant amount of TFAI is present. The bulk temperature may therefore be about 150° C. or less, about 140° C. or less, about 130° C. or less, about 120° C. or less, or about 110° C. or less.

To maintain temperatures at the desired temperatures, tempered water or other heat transfer fluids may be used, or a low-pressure steam may be used. In some instances, a lower boiling compound or compounds may be added in order to keep the bulk temperature below the 150° C. point; this may be particularly useful when pure TFAI is not desired. A lower boiling compound is preferably selected from within the integrated process, such as TFAC, HCl, and/or CO. The bulk temperature may also be limited by selecting the operating pressure below about 137 psig in the distillation steps that contain TFAI, although the pressure may depend on the presence of other compounds in the distillation steps.

Selecting distillation conditions (specifically, low temperature and/or low pressure) for the purification of TFAI may minimize formation of CF₃I. The potential to form CF₃I on hot surfaces (such as reboilers, and other heaters) may be further reduced by using heating media at temperatures below 130° C., such as about 130° C. or lower, about 125° C. or lower, about 120° C. or lower, or about 115° C. or lower.

Using low pressure steam may also reduce the formation of CF₃I. The steam may have a pressure of less than about 30 psig, such as about 30 psig or lower, about 25 psig or lower, about 22 psig or lower, about 20 psig or lower, or about 15 psig or lower.

As a further measure to reduce the formation of CF₃I, intermediate heat transfer fluids may be employed. Suitable fluids may include tempered water and hot oil, for example.

### 5. Azeotrope or Azeotrope-Like Compositions of CF₃I and TFAC

As mentioned above, it has been found that trifluoroacetyl chloride (TFAC) forms homogeneous, minimum boiling azeotrope and azeotrope-like compositions or mixtures with trifluoroiodomethane (CF₃I), and the present disclosure provides homogeneous azeotrope or azeotrope-like compositions comprising TFAC and CF₃I. The azeotrope or azeotrope-like compositions may consist essentially of TFAC and CF₃I, or the azeotrope or azeotrope-like compositions may consist of TFAC and CF₃I.

The azeotrope-like composition of TFAC and CF₃I is a composition or range of compositions which boils at a temperature range of between about −46.0° C. and about 90.0° C. at a pressure of between about 4.9 psia and about 348 psia, including, for example, a composition or range of compositions which boils at a temperature range of about −22.50° C. 0.30° C. at a pressure of about 14.41 psia±0.30 psia.

The azeotrope or azeotrope-like consists essentially of, or consists of, from about 0.5 wt. % to about 99.0 wt. % trifluoroacetyl chloride (TFAC) and from about 1.0 wt. % to about 99.5 wt. % trifluoroiodomethane (CF₃I).

As presented in the Examples below, the pressure sensitivity of the present azeotropic compositions allows the separation of compositions including TFAC and CF₃I to form essentially pure compositions of each of TFAC and CF₃I by “pressure swing” distillation.

One method of separating trifluoroacetyl chloride (TFAC) and trifluoroiodomethane (CF₃I) from a primary composition including TFAC and CF₃I includes the initial step of conveying a feed stream including the primary composition to a low-pressure column. A bottoms product may be collected from the low-pressure column which consists essentially of pure TFAC. A first distillate is then conveyed from the low-pressure column to a high-pressure column via a pump or compressor to increase the pressure, where the first distillate is an azeotrope or azeotrope-like composition consisting essentially of effective amounts of TFAC and CF₃I. A second bottoms product may be collected from the high-pressure column which consists essentially of pure CF₃I. The method may further include, after the second collecting step, the additional step of recycling the second distillate from the high-pressure column back to the feed stream comprising the primary composition.

Similarly, another method of separating TFAC and CF₃I from a primary composition including TFAC and CF₃I includes the initial step of conveying a feed stream including the primary composition to a high-pressure column. A bottoms product may be collected from the high-pressure column which consists essentially of pure CF₃I. A first distillate is then conveyed from the high-pressure column to a low-pressure column, where the first distillate is an azeotrope or azeotrope-like composition consisting essentially of effective amounts of TFAC and CF₃I. A second bottoms product may be collected from the low-pressure column which consists essentially of TFAC. The method may further include, after the second collecting step, the additional step of recycling a second distillate from the low-pressure column via a pump or compressor back to the feed stream comprising the primary composition.

### 6. Breaking the Azeotrope or Azeotrope-Like Compositions of TFAC and CF₃I

The components of the azeotrope or azeotrope-like composition (trifluoroacetyl chloride (TFAC) and trifluoroiodomethane (CF₃I)) may be difficult to separate from one another; in other words, it may be difficult to break the azeotrope or azeotrope-like composition.

One method provided by the present disclosure to break the azeotrope or azeotrope-like composition of trifluoroiodomethane (CF₃I) and trifluoroacetyl chloride (TFAC) comprises contacting the azeotrope or azeotrope-like composition with a solvent, extracting one of the CF₃I and the TFAC into the solvent to form a first composition including the solvent and one of the CF₃I and the TFAC, and a second composition comprising the other of the CF₃I and the TFAC, and separating the first and second compositions. Following separation, the CF₃I and/or the TFAC may be purified.

Specifically, the azeotrope or azeotrope-like composition may be contacted with a solvent, to selectively interact with, or absorb, one of the components of the azeotrope or azeotrope-like composition, resulting in a first composition and a second composition. The first composition comprises a one of the CF₃I and the TFAC and the solvent, depending upon which of these components of the azeotrope or azeotrope-like mixture the solvent selectively interacts with. The second composition comprises the other of the CF₃I and the TFAC. The first and second compositions may then be separated from one another.

As used herein, in connection with breaking the or azeotrope-like compositions, the term “solvent” refers to one or more chemical compounds that selectively interacts with one of the components of the azeotrope or azeotrope-like composition. For example, one of the components of the azeotrope or azeotrope-like composition may be selectively absorbed into the solvent, thereby separating the components of the azeotrope or azeotrope-like composition. Suitable solvents may include sulfur dioxide (SO₂), mineral oil, toluene, acetonitrile, or a combination of two or more of these, for example. Mineral oil refers to a light mixture of higher alkanes from a mineral source, such as a petroleum distillate.

In particular, breaking the azeotrope or azeotrope-like composition occurs upon contacting the azeotrope or azeotrope-like composition with the solvent. This may be accomplished by simple blending of azeotrope or azeotrope-like composition with the solvent, such as by mixing or in a distillation column. Optionally, the blend may be subjected to distillation conditions.

After contacting the azeotrope or azeotrope-like composition with the solvent, sufficient contact time between the azeotrope or azeotrope-like composition allows the mixture to reach equilibrium conditions. Once equilibrium is reached, one component of the azeotrope or azeotrope-like composition will be found predominantly in the solvent, while the other component will be predominantly excluded from the solvent.

Specifically, the ratio of trifluoroiodomethane (CF₃I) to trifluoroacetyl chloride (TFAC) in the solvent may be about 2.0:1.0 or greater, about 2.5:1.0 or greater, about 3.0:1.0 or greater, about 3.5:1.0 or greater, about 5.0:1.0 or greater, about 10.0:1.0 or greater, about 100:1.0 or greater, or about 1000:1.0 or greater. Alternatively, the ratio of trifluoroacetyl chloride (CF₃COCl) to trifluoroiodomethane (CF₃I) in the solvent may be about 2.0:1.0 or greater, about 2.5:1.0 or greater, about 3.0:1.0 or greater, about 3.5:1.0 or greater 5.0:1.0 or greater, about 10.0:1.0 or greater, about 100:1.0 or greater, or about 1000:1.0 or greater.

Following the addition of the solvent, several possible methods exist by which the trifluoroacetyl chloride (TFACl) and trifluoroiodomethane (CF₃I) may be separated from one another, including pressure swing distillation, azeotropic extraction, liquid-liquid extraction, absorption or extractive distillation, for example.

In one example, the present disclosure provides a method to separate the components of the azeotrope or azeotrope-like composition (trifluoroacetyl chloride (TFAC) and trifluoroiodomethane (CF₃I)) using extractive distillation. The azeotrope or azeotrope-like composition may be fed to an extractive distillation column. A solvent may be fed to the extractive distillation column, such that the solvent contacts the azeotrope or azeotrope-like composition, resulting in a first composition comprising one of the trifluoroiodomethane (CF₃I) and the trifluoroacetyl chloride (TFAC) which comprises a first distillate, and a second composition comprising the other of the trifluoroiodomethane (CF₃I) or the trifluoroacetyl chloride (TFAC) and the solvent, which comprises a first bottoms product.

The first distillate may be recycled back to a prior process flow and/or may be subjected to purification. The first bottoms product may then be passed to a distillation column to produce a second distillate and a second bottoms product. The second distillate comprises a product stream of one of purified CF₃I or purified TFAC. The second bottoms product comprises recovered solvent, which may be purged. Alternatively, the recovered solvent may be recycled, first via an optional cooler to reduce the temperature of the solvent. Additional solvent may be added if necessary, and the recovered solvent and additional solvent may be passed to an optional solvent recovery vessel. From the optional solvent recovery vessel, the solvent may pass through a solvent recycle pump to join the solvent stream prior to being fed to the extractive distillation column.

The extractive distillation column may be a single-stage flash column. Alternatively, the extractive distillation column may be a multi-stage column.

In one example of the method discussed above, the first composition may comprise TFAC and the second composition may comprise CF₃I and the solvent. Following extractive distillation, the first distillate may comprise TFAC, which may be recycled back to the reactor. The first bottoms product, comprising CF₃I and the solvent may then be passed to a distillation column to produce a second distillate comprising a product stream of CF₃I and a second bottoms comprising recovered solvent, which may be purged or recycled back to the extractive distillation column. In a further step, the CF₃I may optionally be purified.

7. Removal and Recovery of Iodine (I₂) from Trifluoroacetyl Iodide Feed Stream and Trifluoroiodomethane Product Stream

The presence of iodine (I₂)-containing impurities in the form of both iodine (I₂) and HI₃ in the trifluoroacetyl iodide (TFAI) may lead to operational issues. Specifically, the presence of these iodinated impurities may increase the formation of unwanted by-products, such as trifluoromethane, which may form during the conversion step of TFAI to trifluoroiodomethane (CF₃I) due to the presence of hydrogen-containing species, such as HI and HI₃, thereby lowering the overall process yield and possibly causing difficulties in purification of the trifluoroiodomethane final product. Additionally, the presence of iodine can lead to deposits of solid material within equipment and piping that can lead to plugging or fouling.

The present disclosure provides various methods to remove iodine at different points in the process. For example, the iodine may be removed from the TFAI feedstock upstream of the reactor or from the CF₃I product stream downstream of the reactor or both.

In one method, the TFAI feedstock may be passed through column charged with carbonaceous materials to remove HI, hydrogen triiodide (HI₃) and iodine from the feedstock.

The present disclosure also provides a method wherein at least one column is used to remove iodine-containing products from the reactor effluent stream. This column may be positioned such that iodine-containing species, such as HI₃ and I₂, may be removed from the CF₃I product stream from Equation 3 above. In one method, a solvent may be added to the reactor effluent to keep iodine soluble in order to prevent iodine from depositing on surfaces. Limiting the formation of iodine solids may limit operational issues, such as plugging and corrosion.

As discussed above, suitable solvents are those with high iodine solubility, such as benzene and alkyl-substituted benzenes. Solvents may include benzene, toluene, xylenes, mesitylene (1,3,5-trimethylbenzene), ethyl benzene and the like; dimethylformamide (DMF), dimethyl sulfoxide, (DMSO), and ionic liquids such as imidazolium salts and caprolactamium hydrogen sulfate, for example, and combinations thereof.

Referring to FIGS. 2, 2A, and 3, in one method, iodine-containing products may be removed from the CF₃I product stream using the method described below. A feed stream comprising TFAI may be passed through a reactor 200 to provide a product stream 202 comprising CF₃I, TFAI, carbon monoxide (CO), trifluoroacetic acid (TFA), hydrogen triiodide (HI₃), and iodine (I₂). A solvent such as toluene may be added to the product stream 202 prevent solid iodine from depositing on surfaces. A stream (138 in FIG. 2 or 126A in FIG. 2A) derived from the formation of TFAI (the previous step of the integrated process), comprising crude trifluoroacetyl iodide (TFAI), iodine (I₂) and HI₃, may be combined with the product stream 202 before it is conveyed to a first column 204 to provide a first overhead product stream 208 comprising CF₃I, CO, and small amounts of low-boiling impurities. The first overhead product stream 208 may be further processed to provide refrigerant grade CF₃I. A first bottoms product stream 206 comprising unreacted TFAI, iodine, HI₃, and high-boiling components may be combined with a stream 112 (in FIG. 2 and FIG. 2A) from the previous step of the integrated process comprising crude TFAI, iodine and HI₃. The combined stream is then fed to a second column 124 (FIG. 2 and FIG. 2A) and processed as described above. The iodine removal processes of the present disclosure, such as the example described above, may be run as a continuous process or may be conducted as an intermittent process.

The columns are operated under conditions preventing or minimizing reactions between organic constituents, such as TFAI, TFA, etc., iodine (I₂) and the solvent used.

The overhead of the second column (column 124 in FIG. 2) may be operated at a temperature of from about 0° C. to about 150° C., for example at about 0° C. or higher, about 20° C. or higher, about 40° C. or higher, about 60° C. or higher, about 80° C. or higher, about 100° C. or higher, about 125° C. or higher, about 150° C. or lower, about 125° C. or lower, about 100° C. or lower, about 80° C. or lower, about 60° C. or lower, about 40° C. or lower, about 20° C. or lower, about 10° C. or lower, or within any range defined between any two of the foregoing values or any value encompassed by these endpoints.

The bottoms temperature of the second column may be operated at a temperature of from about 60° C. to about 260° C., for example at about 60° C. or higher, about 90° C. or higher, about 120° C. or higher, about 150° C. or higher, about 180° C. or higher, about 210° C. or higher, about 240° C. or higher, about 260° C. or lower, about 240° C. or lower, about 210° C. or lower, about 180° C. or lower, about 150° C. or lower, about 120° C. or lower, about 90° C. or lower, or within any range defined between any two of the foregoing values or any value encompassed by these endpoints.

The second column may be operated at a pressure of about −10 psig to 250 psig, for example at about 0 psig or higher, about 50 psig or higher, about 100 psig or higher, about 150 psig or higher, about 200 psig or higher, about 250 psig or less, about 200 psig or less, about 150 psig or less, about 100 psig or less, about 50 psig or less, about 0 psig or less, or within any range defined between any two of the foregoing values or any value encompassed by these endpoints.

The overhead of the third column (column 130 in FIG. 2) may be operated at a temperature of from about 60° C. to about 250° C., for example at about 60° C. or higher, about 90° C. or higher, about 120° C. or higher, about 150° C. or higher, about 180° C. or higher, about 210° C. or higher, about 240° C. or higher, about 250° C. or lower, about 240° C. or lower, about 210° C. or lower, about 180° C. or lower, about 150° C. or lower, about 120° C. or lower, about 90° C. or lower, or within any range defined between any two of the foregoing values or any value encompassed by these endpoints.

The bottoms temperature of the third column may be operated at a temperature of from about 135° C. to about 350° C., for example at about 150° C. or higher, about 200° C. or higher, about 250° C. or higher, about 300° C. or higher, about 325° C. or higher, about 350° C. or lower, about 325° C. or lower, about 300° C. or lower, about 250° C. or lower, about 200° C. or lower, about 150° C. or lower, or within any range defined between any two of the foregoing values or any value encompassed by these endpoints.

The third column may be operated at a pressure of from about −10 psig to about 200 psig, for example at about 0 psig or higher, about 50 psig or higher, about 100 psig or higher, about 150 psig or higher, about 200 psig or less, about 150 psig or less, about 100 psig or less, about 50 psig or less, about 0 psig or less, or within any range defined between any two of the foregoing values or any value encompassed by these endpoints.

The overhead of the fourth column (column 136 in FIG. 2) may be operated at a temperature of from about 0° C. to about 150° C., for example at about 0° C. or higher, about 20° C. or higher, about 40° C. or higher, about 60° C. or higher, about 80° C. or higher, about 100° C. or higher, about 125° C. or higher, about 150° C. or lower, about 125° C. or lower, about 100° C. or lower, about 80° C. or lower, about 60° C. or lower, about 40° C. or lower, about 20° C. or lower, about 10° C. or lower, or within any range defined between any two of the foregoing values or any value encompassed by these endpoints.

The bottoms temperature of the fourth column may be maintained at a temperature below of from about 55° C. to 250° C., for example at about 60° C. or higher, about 90° C. or higher, about 120° C. or higher, about 150° C. or higher, about 180° C. or higher, about 210° C. or higher, about 240° C. or higher, about 250° C. or lower, about 240° C. or lower, about 210° C. or lower, about 180° C. or lower, about 150° C. or lower, about 120° C. or lower, about 90° C. or lower, about 90° C. or lower, or within any range defined between any two of the foregoing values or any value encompassed by these endpoints.

The fourth column may be operated at a pressure of from about −10 psig to about 200 psig, for example at about 0 psig or higher, about 50 psig or higher, about 100 psig or higher, about 150 psig or higher, about 200 psig or less, about 150 psig or less, about 100 psig or less, about 50 psig or less, about 0 psig or less, or within any range defined between any two of the foregoing values or any value encompassed by these endpoints.

In an alternative embodiment, as shown in FIG. 2A, the overhead of the second column (column 124A in FIG. 2A) may be operated at a temperature of from about 0° C. to about 150° C., for example at about 0° C. or higher, about 20° C. or higher, about 40° C. or higher, about 60° C. or higher, about 80° C. or higher, about 100° C. or higher, about 125° C. or higher, about 150° C. or lower, about 125° C. or lower, about 100° C. or lower, about 80° C. or lower, about 60° C. or lower, about 40° C. or lower, about 20° C. or lower, about 10° C. or lower, or within any range defined between any two of the foregoing values or any value encompassed by these endpoints.

The bottoms temperature of the second column may be operated at a temperature of from about 60° C. to about 250° C., for example at about 60° C. or higher, about 90° C. or higher, about 120° C. or higher, about 150° C. or higher, about 180° C. or higher, about 210° C. or higher, about 240° C. or higher, about 250° C. or lower, about 240° C. or lower, about 210° C. or lower, about 180° C. or lower, about 150° C. or lower, about 120° C. or lower, about 90° C. or lower, or within any range defined between any two of the foregoing values or any value encompassed by these endpoints.

The second column may be operated at a pressure of from about −10 psig to about 250 psig, for example at about 0 psig or higher, about 50 psig or higher, about 100 psig or higher, about 150 psig or higher, about 200 psig or higher, about 250 psig or less, about 200 psig or less, about 150 psig or less, about 100 psig or less, about 50 psig or less, about 0 psig or less, or within any range defined between any two of the foregoing values or any value encompassed by these endpoints. {0018-1} The overhead of the third column (column 130A in FIG. 2A) may be operated at a temperature of from about 60° C. to about 200° C., for example at about 60° C. or higher, about 90° C. or higher, about 120° C. or higher, about 150° C. or higher, about 180° C. or higher, about 200° C. or lower, about 180° C. or lower, about 150° C. or lower, about 120° C. or lower, about 90° C. or lower, or within any range defined between any two of the foregoing values or any value encompassed by these endpoints.

The bottoms temperature of the third column may be operated at a temperature of from about 70° C. to about 350° C., for example at about 100° C. or higher at about 150° C. or higher, about 200° C. or higher, about 250° C. or higher, about 300° C. or higher, about 325° C. or higher, about 350° C. or lower, about 325° C. or lower, about 300° C. or lower, about 250° C. or lower, about 200° C. or lower, about 150° C. or lower, about 100° C. or lower, or within any range defined between any two of the foregoing values or any value encompassed by these endpoints.

The third column may be operated at a pressure of from about −10 psig to about 200 psig, for example at about 0 psig or higher, about 50 psig or higher, about 100 psig or higher, about 150 psig or higher, about 200 psig or less, about 150 psig or less, about 100 psig or less, about 50 psig or less, about 0 psig or less, or within any range defined between any two of the foregoing values or any value encompassed by these endpoints.

The overhead of the fourth column (column 136A in FIG. 2A) may be operated at a temperature of about 40° C. to 150° C., for example at about 60° C. or higher, about 80° C. or higher, about 100° C. or higher, about 125° C. or higher, about 150° C. or lower, about 125° C. or lower, about 100° C. or lower, about 80° C. or lower, about 60° C. or lower, or within any range defined between any two of the foregoing values or any value encompassed by these endpoints.

The bottoms temperature of the fourth column may be maintained at a temperature below of from about 65° C. to about 300° C., for example at about 70° C. or higher, about 90° C. or higher, about 120° C. or higher, about 150° C. or higher, about 180° C. or higher, about 210° C. or higher, about 250° C. or higher, about 300° C. or lower about 250° C. or lower, about 210° C. or lower, about 180° C. or lower, about 150° C. or lower, about 120° C. or lower, about 90° C. or lower, about 70° C. or lower, or within any range defined between any two of the foregoing values or any value encompassed by these endpoints.

The fourth column may be operated at a pressure of from about −10 psig to about 200 psig, for example at about 0 psig or higher, about 50 psig or higher, about 100 psig or higher, about 150 psig or higher, about 200 psig or less, about 150 psig or less, about 100 psig or less, about 50 psig or less, about 0 psig or less, or within any range defined between any two of the foregoing values or any value encompassed by these endpoints.

Another alternative for removing iodine from a vapor stream using vapor/liquid contacting columns may allow for the iodine to be recovered as a liquid, which provides an advantage over recovery of solid iodine as it does not need to be melted off of any equipment and does not cause issues such as plugging.

In this method, a feed stream comprising the components to be recovered, such as TFAI and TFA, for example, may be fed to a first column, along with a solvent including a low concentration of iodine. The first column includes a condenser and rectification section to allow for reflux, and may optionally include a reboiler and stripping section. A first overhead product stream, which includes vapor from the first column, may contain the component to be recovered, such as TFAI. A first bottoms product stream may include a solvent and iodine. The first bottoms product stream may be conveyed to a second column, which includes a reboiler and a stripping section and, optionally, a condenser and a rectification section. A second overhead product stream may include solvent in the form of a vapor or a liquid. This overhead product stream may be recycled back to the first column, along with optional fresh solvent. A second bottoms product stream from the second column may include liquid iodine, which may be recovered.

Optionally, further columns may be included to conduct partial separations.

The solvent in the method described above may be a solvent with high solubility of iodine. The solvent may have a vapor pressure higher than that of iodine but lower than that of the components being recovered in the gas stream. Suitable solvents may include benzene; xylenes, such as paraxylene, and metaxylene; alkylated benzenes, such as mesitylene (1,3,5-trimethylbenzene), toluene, ethyl benzene; dimethylformamide (DMF); and dimethyl sulfoxide (DMSO), for example.

The solvent type, solvent circulation rate, first column pressure, and first column reboiler heat input are selected such that the iodine does not form a solid phase. For example, the temperature may be above 114° C., the melting point of iodine (I₂). This permits the first overhead product to be substantially free of iodine (I₂) as the iodine is dissolved in the solvent and exits the first column in the bottom product.

The first overhead product stream may contain iodine in an amount of about 10,000 ppm or less, about 7000 ppm or less, about 5000 ppm or less, about 2500 ppm or less, about 1000 ppm or less, about 500 ppm or less, about 100 ppm or less, about 50 ppm or less, about 10 ppm or less, about 1 ppm or less, or about 0 ppm. This permits the first overhead product stream to be substantially free of iodine as the iodine is dissolved in the solvent and exists the first column in the bottoms product stream.

The solvent type, solvent circulation rate, second column pressure, and second column reboiler heat input are selected such that the iodine (I₂) does not form a solid phase. For example, the temperature may be above 114° C., the melting point of iodine (I₂). This permits the second overhead product stream to be substantially free of iodine as the iodine is present as a liquid and exits the second column as the bottoms product stream. The operating pressure of the second column may be lower than that of the first column.

The second overhead product stream may contain iodine in an amount of about 10,000 ppm or less, about 7000 ppm or less, about 5000 ppm or less, about 2500 ppm or less, about 1000 ppm or less, about 500 ppm or less, about 100 ppm or less, about 50 ppm or less, about 10 ppm or less, about 1 ppm or less, or about 0 ppm.

As yet another alternative, liquid iodine may be recovered from a component to be recovered via a phase separation using a third component. Suitable third components may be compatible with the reaction and recovery process, may be miscible with the organic components present, and may be substantially immiscible with iodine. One such component is TFA.

A mixture of iodine and TFA may be heated to a temperature above the melting point of iodine to maintain it in the liquid phase. The mixture may then be allowed to settle into two layers. The top organic layer may be decanted to recover the desired products. Preferably, TFA is separated from TFAI by distillation or series of distillation steps for recycle. The bottom layer comprising liquid iodine may then be recycled back to the first step of the integrated process or may be stored for alternate use. Optionally, the iodine may be further purified.

The temperature may be about 114° C. or higher, about 115° C. or higher, about 120° C. or higher, about 125° C. or lower, about 130° C. or lower, about 135° C. or lower, about 140° C. or lower, or any value encompassed by these endpoints.

As another alternative, the crude CF₃I product stream comprising TFAI, CF3I, HI3 and iodine may be passed through a column charged with carbonaceous materials to remove hydrogen triiodide (HI₃) and iodine from the crude product.

8. Formation of Trifluoroiodomethane (CF₃I) from Trifluoroacetyl Iodide (TFAI)

As discussed above, in the third reaction step of the integrated process, trifluoroacetyl iodide (TFAI) is reacted to form trifluoroiodomethane (CF₃I) and carbon monoxide (CO). The present disclosure provides gas-phase processes for producing trifluoroiodomethane (CF₃I).

The process comprises providing a reactant stream comprising TFAI, providing the stream to a reactor, optionally contacting the stream with a catalyst, and converting the stream in a reactor to produce a product stream comprising the CF₃I.

When a catalyst is used, the catalyst may comprise stainless steel, nickel, nickel-chromium-molybdenum alloy, nickel-copper alloy, copper, alumina, silicon carbide, platinum, palladium, rhenium, activated carbon, or combinations thereof. The catalyst may comprise activated carbon.

The reaction temperature may be about 200° C. or higher, about 250° C. or higher, about 300° C. or higher, about 350° C. or higher, about 400° C. or higher, about 450° C. or lower, about 500° C. or lower, about 550° C. or lower, about 600° C. or lower, or any value encompassed by these endpoints. Preferably, the reaction may be carried out at a temperature from about 300° C. to about 500° C. More preferably, the reaction may be carried out at temperature from about 300° C. to about 400° C.

The reaction may be carried out at a pressure of about 0 psig or greater, about 5 psig or greater, about 20 psig or greater, about 50 psig or greater, about 70 psig or greater, about 100 psig or greater, about 150 psig or lower, about 200 psig or lower, about 225 psig or lower, about 250 psig or lower, about 275 psig or lower, about 300 psig, or within any range encompassing these endpoints. However, any pressure, such as sub-atmospheric or super-atmospheric pressures may be used in the reaction.

The contact time of the reactant stream with the catalyst may be about 0.1 second or longer, about 1 second or longer, about 5 seconds or longer, about 10 seconds or longer, about 20 seconds or longer, about 30 seconds or longer, about 40 seconds or longer, about 50 seconds or less, about 60 seconds or less, about 80 seconds or less, about 100 seconds or less, about 120 seconds or less, about 180 seconds or less, or any value encompassed by these endpoints.

The process may be a continuous process. The process may further comprise the additional steps of separating unreacted TFAI from the product stream, and returning the separated unreacted TFAI to the reactant stream. The process may further comprise the additional step of separating CO from the product stream. The process may further comprise the additional step of condensing and collecting CF₃I as crude product.

The concentration of CF₃I in the CF₃I crude product may be greater than 99 wt. %, such as about 99 wt. % or greater, about 99.5 wt. % or greater, or about 99.9 wt. % or greater.

In one method, a purified reactant TFAI stream from the second step of the reaction may be vaporized and superheated to the reaction temperature. In some embodiments, a lower boiling compound preferably selected from within the integrated process, such as CF₃I and/or CO may be fed to the vaporizer to reduce the dewpoint of the vaporizing mixture, allowing for lower temperature operation which may reduce formation of iodine.

The reaction may take place in a heated tube reactor or an electric heater reactor. The electric heater reactor may be an impedance tube reactor with the electrical current passing directly through the heater tube wall utilizing alternating current at low voltage. Alternatively, the electric heater reactor may be an immersion-type electric heater. This novel immersion-type electric heater may be a system using electricity as the heating medium, with the reaction occurring on the outside of the heating elements. In another method, a shell and tube reactor with heat transfer medium flowing on the outside of tubes and reactor feed flowing through the tubes may also be suitable. An impedance heater where the reactor tubes are heated directly by electricity may also be used. The impedance reactor may be comprised of tubes or pipes, such as found in a shell and tube configuration. In an embodiment, one or more of the tubes or pipes may be finned, while in another embodiment, none of the tubes of the tubes or pipes are finned. Thus, in an embodiment, all of the tubes or pipes are smooth, while in another embodiment, at least one of the tubes or pipes is smooth. The electrical current may be passed through the surface of the pipes and/or through packing disposed inside or outside the pipes or otherwise in the reactor in order to provide reactor heating.

The reactor may comprise a metal alloy which encases Nichrome heating elements within compacted magnesium oxide (MgO) powder. In some embodiments, multiple units may be used in series and/or in parallel.

The reactor may comprise a metal alloy, such as Inconel® 600, Inconel® 625, Incoloy® 800 and Incoloy® 825, for example.

The heater surface may be a catalytic surface or a non-catalytic surface. Suitable metal surfaces may include electroless nickel, nickel, stainless steel, nickel-copper alloy, nickel-chromium-iron alloy, nickel-chromium alloy, nickel-chromium-molybdenum alloy, or combinations thereof.

The reaction temperature may be about 200° C. or higher, about 250° C. or higher, about 300° C. or higher, about 350° C. or higher, about 400° C. or higher, about 450° C. or lower, about 500° C. or lower, about 550° C. or lower, about 600° C. or lower, or any value encompassed by these endpoints. Preferably, the reaction may be carried out at a temperature from about 300° C. to about 500° C. More preferably, the reaction may be carried out at temperature from about 300° C. to about 400° C.

The reaction may be carried out at a pressure of about 0 psig or greater, about 5 psig or greater, about 20 psig or greater, about 50 psig or greater, about 70 psig or greater, about 100 psig or greater, about 150 psig or lower, about 200 psig or lower, about 225 psig or lower, about 250 psig or lower, about 275 psig or lower, about 300 psig, or within any range encompassing these endpoints. However, any pressure, such as sub-atmospheric or super-atmospheric pressures may be used in the reaction.

The reactant stream may be in contact with the heater for a period of time of about 0.1 seconds or greater, about 1 second or greater, about 5 seconds or greater, about 10 seconds or greater, about 20 seconds or greater, about 30 seconds of greater, about 40 seconds or greater, about 50 seconds or less, about 60 seconds or less, about 80 seconds or less, about 100 seconds or less, about 120 seconds or less, about 180 seconds or less, or any value encompassed by these endpoints.

Optionally, the reactor effluent (or crude product stream) may be used to heat vaporized TFAI in order to conserve energy.

During the decomposition of TFAI to form CF₃I and CO, the conversion per pass may be about 10% or greater, about 20% or greater, about 30% or greater, about 40% or greater, about 50% or greater, about 60% or greater, about 70% or greater, about 80% or greater, or about 90% or greater. The conversion is chosen such that a balance may be achieved between equipment size and selectivity.

Regarding equipment size, low conversion per pass leads to greater recycling and requires larger equipment.

Regarding selectivity towards undesired side products, higher conversion per pass may yield more undesired side products.

### 9. Purification of Trifluoroiodomethane (CF₃)

The product stream may comprise mostly CF₃I and CO. The reactant stream may also comprise further by-products such as carbon dioxide (CO₂), TFA, and organohalides such as R23 (CH₃F), R13 (CClF₃), trifluoroacetyl fluoride (TFAF), trifluoroacetic acid (TFA), pentafluoropropanone, 133a (2-chloro-1,1,1-trifluoroethane), pentafluoroiodoethane (C₂F₅I), methyl propane (also known as isobutane CH(CH₃)₃) as well as iodine. The reactor effluent may also include unreacted TFAI.

The present disclosure provides methods to purify the CF₃I product using distillation columns. In this method, the reactor effluent is optionally cooled and may be fed to a first distillation column to provide a first overhead product stream and a first bottoms product stream. Optionally, toluene may be added to the reactor effluent before it enters the first column to prevent iodine from solidifying in the piping or inside the column. The first overhead product stream may comprise CF₃I, CO and other low-boiling components, including pentafluoroiodoethane (CF₃CF₂I), TFAF, R13, and R23, methyl propane, for example.

The first bottoms product stream may contain unreacted TFAI, as well as higher-boiling components, such as TFA and I₂, for example. If toluene is added to the stream prior to entering the column, it may comprise a portion of the bottoms product stream. The bottoms product stream may be combined with the recycle streams discussed above.

The first overhead product stream may be compressed in a compressor and fed to a second distillation column to provide a second overhead product stream and a second bottoms product stream. An HCl stream from distillation columns used at the other points along the process (as discussed above) may also be fed to the second distillation column. The second overhead product stream may comprise CO and HCl. The second overhead product stream may optionally be passed over an adsorbent to remove any residual acidic components other than HCl, such as HI, and residual organics, such as TFAF, and then absorbed into water to form aqueous HCl. Feeding HCl to the second column may allow reflux to begin at higher temperature than when the second overhead product stream comprises mostly CO, which has a very low boiling point requiring a combination of high pressure and very low temperature coolant. Thus, it will be appreciated that the instant process does not require the otherwise necessary cryogenic condensing of CO or yet a further distillation involving extractive distillation with another reagent in combination with high pressure due to its low boiling point. In some embodiments, HCl produced from one or more different processes may also be used to feed to the second column for the purpose of generating reflux in the presence of CO.

One method provided by the present disclosure to remove acidic by-products from the CF₃I product stream may include feeding the reactor effluent stream to an acid absorption system wherein the gaseous stream may be contacted with water or a basic solution to form HF, HCl, and HI (or corresponding halide salts), and TFA.

The purification may be performed in a continuous fashion. The reactor effluent stream comprising CF₃I, fluorinated- and iodinated-hydrocarbons, CO₂, mineral acids, and water may be contacted with a mild caustic solution to neutralize mineral- and organic-acids. It is desirable to avoid high concentrations of caustic components to limit both the decomposition of CF₃I and precipitation of metals salts in the scrubbing system. For example, mildly basic solutions of alkali earth metal hydroxides or carbonates in water may be used. Suitable mildly basic solutions may include 0.5 wt. % sodium hydroxide (NaOH) in water or 0.5 wt. % potassium hydroxide (KOH) in water, for example.

The mildly basic solution may be used to neutralize acids in the reactor effluent stream. The reactor effluent stream can be contacted with the solution using several different techniques. In one example, a scrubbing tower may be used with a co-current flows or countercurrent flows.

As a further example, the contact may occur in a vessel where the reactor effluent stream is bubbled through as a gas, at an appropriate contact time, temperature and pressure. Operationally, the contact time is about 30 seconds, and the scrubbing is performed at ambient temperature and pressure, leading to material containing less than 1 ppm of total acid content.

Alternatively, the scrubbing system may be replaced with an adsorption column containing a suitable adsorbent to remove acids. Suitable adsorbents may include alumina, activated charcoal, carbides, nitrides, zirconias, and silica. It is desirable that the adsorbent used selectively removes the acids without initiating or favoring decomposition of CF₃I. Neither alumina P188 nor alumina CLR-204 promote the decomposition of CF₃I, and therefore are suitable for removal of acids.

Within the adsorption column, several different types of adsorbents may be used at the same time. The adsorbents may be mixed or may be layered consecutively. Removal of acid using the adsorbent column may be performed with the material flowing through it either as a liquid or a gas, at a suitable temperature and pressure. When P188 and CLR-204 are used consecutively in an adsorbent column, greater than 90% reduction in total acid content may observed.

If desired, the effluent stream from the scrubbing system or adsorbent column may then be passed to a drying column containing a suitable desiccant for water removal. Several desiccants can be used for this application, such as molecular sieves, anhydrous calcium chloride, anhydrous calcium sulfate, concentrated sulfuric acid, silica, activated charcoal and zeolites, for example. It is desirable that the desiccant does not promote secondary reaction pathways favoring the decomposition of CF₃I, as that would reduce the overall yield of the purification. One such option is the use of 3 A molecular sieves as these are compatible with CF₃I.

Desiccants have a finite capacity for adsorbing moisture and after normal use will have reduced or no discernible adsorption capacity. Desiccants such as molecular sieves, calcium sulfate and others may be regenerated for repetitive use, for example, as described below for molecular sieves. It should be understood that same or similar procedure may be applied to other desiccants such as calcium sulfate.

Recovery of residual CF₃I may be accomplished by draining out the residual CF₃I as a liquid or by venting off CF₃I as vapor. This initial CF₃I recovery may optionally be conducted under vacuum and/or heating, for example, via a jacket on the adsorption column, heating to about 100° C. to provide additional driving force to speed up removing the residual CF₃I from the adsorber.

After recovering CF₃I as described above, the molecular sieves may be regenerated by passing hot inert gas such as nitrogen or air over the molecular sieve bed. The adsorber is heated by the hot inert gas in a progressive and incremental manner to a temperature of about 230° C. or higher to desorb the remaining CF₃I, followed by desorption of water from the molecular sieves. This progressive and incremental set of temperature increases and holds allow the remaining CF₃I to be desorbed from the molecular sieves at a lower temperature, prior to desorbing the bulk of the water at a higher temperature.

Following regeneration, the bed is cooled and preferentially evacuated to remove the non-condensable inert gas used for regeneration in order to be ready for the next water adsorption cycle. Evacuation of non-condensables minimizes or prevents introduction of non-condensables into downstream processing steps, which would lead to lowered yields.

The effluent stream from the drying column may be collected in a crude storage tank. The material may then in turn be fed to a first distillation column, in which carbon monoxide (CO) and volatile organic components may be removed as a first overhead product stream, while CF₃I and higher boiling components may be concentrated in the reboiler. The contents of the reboiler may then be passed to a second distillation column in which CF₃I may be collected as the second overhead product stream and higher boiling components may be accumulated in the reboiler.

The second overhead product stream may comprise CF₃I in an amount of about 95 wt. % or greater, about 99 wt. % or greater, 99.5 wt. % or greater, 99.9 wt. % or greater, or 99.99 wt. % or greater.

The acid content of the CF₃I in the second overhead product stream may be about 0.1 wt. % or less, about 0.01 wt. % or less, about 0.001 wt. % or less, or about 0.0001 wt. % or less.

The water content of the CF₃I in the second overhead product stream may be about 10 wt. % or less, about 5 wt. % or less, about 1 wt. % or less, about 0.5 wt. % or less, about 0.1 wt. % or less, about 0.01 wt. % or less, about 0.001 wt. % or less, or about 0.0001 wt. % or less.

It is not necessary to perform these purification processes in the sequence described above. For example, the distillation process may be performed prior to or following the acid and water removal steps. Independent of the sequence used, the CF₃I material obtained may be of the high purity described above.

Alternatively, a sulfuric acid drying system could be used place of, or in addition to, drying using molecular sieves as described above. In one such method, a feed stream comprising CF₃I and water may be contacted with a concentrated sulfuric acid solution. It has surprisingly been found that, although many hydrofluoroolefins (HFO's) undergo decomposition when exposed to sulfuric acid, CF₃I and the mixtures of the present disclosure containing CF₃I undergo minimal or no decomposition in the presence of sulfuric acid.

In this method, water may be preferentially absorbed into the sulfuric acid, resulting in a product stream of CF₃I that is substantially free of water. The feed stream comprising CF₃I and water may be contacted by the sulfuric acid in a contacting tower in which the feed stream comprising CF₃I and water may be present as vapor flowing in a countercurrent manner to the liquid sulfuric acid. For efficiency, a circulating system may be used for the sulfuric acid.

The amount of water in the product stream of CF₃I may be about 20 ppm or less, about 15 ppm or less, about 10 ppm water or less, about 5 ppm water or less, or about 1 ppm water or less.

As a further alternative, a feed stream comprising CF₃I and water may be condensed at a temperature and pressure combination to permit condensation of water without freezing it. The resulting mixture may be allowed to settle, and the water layer (if one is present) may be decanted off. The organic layer may then be fed to a distillation column from which a heterogeneous mixture, such as a simple mixture of CF₃I and water or an azeotropic or azeotrope-like mixture of CF₃I and water may be collected as the overhead product stream, and a bottoms product stream comprising CF₃I may be collected.

The bottoms product stream comprising CF₃I may be substantially free of water. Specifically, the amount of water in the bottoms product stream may be about 20 ppm or less, about 15 ppm or less, about 10 ppm water or less, about 5 ppm water or less, or about 1 ppm water or less.

In yet another method, a feed stream comprising CF₃I and water may be contacted with a desiccant to provide a product stream comprising CF₃I that is substantially free of water. Suitable desiccants may include 3 Angstrom molecular sieves, 4 Angstrom molecular sieves, 5 Angstrom molecular sieves, activated alumina, silica gel, calcium sulfate (“Drierite”), and calcium chloride, for example.

The feed stream may be a vapor or a liquid.

The amount of water in the product stream may be about 200 ppm or less, about 170 ppm or less, about 150 ppm or less, about 100 ppm or less, about 50 ppm or less, about 30 ppm or less, about 20 ppm or less, about 15 ppm or less, about 10 ppm or less, about 5 ppm or less, or about 1 ppm or less.

To further purify the CF₃I, a feed stream comprising the product stream from acid absorption and drying comprising CF₃I may be condensed and fed to a first distillation column to provide a first overhead product stream and a first bottoms product stream. The first overhead product stream may comprise impurities with lower boiling points than that of CF₃I. The first overhead product stream may be sent to a thermal oxidizer for disposal. The first bottoms product stream may be sent to a second distillation column to provide a second overhead product stream and a second bottoms product stream. The second overhead product stream may comprise purified CF₃I. The second bottoms product stream may comprise high-boiling compounds, which may be removed as a vapor or liquid and may be disposed of, for example by thermal oxidation.

As a further alternative, the present disclosure provides a method of forming an azeotrope or azeotrope-like composition comprising, consisting essentially of, or consisting of effective amounts of trifluoroiodomethane (CF₃I) and water, which may be used to separate impurities. Once the impurities have been removed, the CF₃I and water may be separated from one another as further described below.

The azeotrope or azeotrope-like composition may comprise from about 47.7 wt. % to about 99.0 wt. % trifluoroiodomethane (CF₃I) and from about 1.0 wt. % to about 52.3 wt. % water, from about 60.4 wt. % to about 95.0 wt. % trifluoroiodomethane (CF₃I) and from about 5.0 wt. % to about 39.6 wt. % water, from about 70.2 wt. % to about 90.0 wt. % trifluoroiodomethane (CF₃I) and from about 10.0 wt. % to about 29.8 wt. % water, or the azeotrope or azeotrope-like composition may consist essentially of about 77.0 wt. % trifluoroiodomethane (CF₃I) and about 23.0 wt. % water. The azeotrope or azeotrope-like composition may consist essentially of trifluoroiodomethane (CF₃I) and water in the above amounts or consist of trifluoroiodomethane (CF₃I) and water in the above amounts.

The azeotrope of azeotrope-like composition has a boiling point between about 18.0° C. and about 19.0° C. at a pressure of between about 58.0 psia and about 60.0 psia.

The present disclosure also provides a method of forming an azeotrope or azeotrope-like composition comprising the step of combining trifluoroiodomethane (CF₃I) and water to form an azeotrope or azeotrope-like composition comprising, consisting essentially of, or consisting of trifluoroiodomethane (CF₃I) and water. The azeotrope of azeotrope-like composition may have a boiling point between about 18.0° C. and about 19.0° C. at a pressure of between about 58.0 psia and about 60.0 psia.

The present disclosure further provides a method of separating impurities from a composition which includes trifluoroiodomethane (CF₃I), water, and at least one impurity, comprising the steps of modifying the relative amounts of trifluoroiodomethane (CF₃I) and water and subjecting the composition to conditions effective to form an azeotrope or azeotrope-like composition consisting essentially of, or consisting of, effective amounts of trifluoroiodomethane (CF₃I) and water; and separating the azeotrope or azeotrope-like composition from the at least one impurity, wherein the separation step may comprise at least one of phase separation, distillation, and fractionation.

The present disclosure further provides a method of separating impurities from a composition which includes trifluoroiodomethane (CF₃I) and at least one impurity, comprising the steps of adding an effective amount of water to the composition; modifying the relative amounts of trifluoroiodomethane (CF₃I) and water and subjecting the composition to conditions effective to form an azeotrope or azeotrope-like composition consisting essentially of, or consisting of, effective amounts of trifluoroiodomethane (CF₃I) and water; and separating the azeotrope or azeotrope-like composition from the at least one impurity, wherein the separation step may comprise at least one of phase separation, distillation, and fractionation.

In the foregoing methods, the step of modifying the relative amounts of trifluoroiodomethane (CF₃I) and water may involve adding trifluoroiodomethane (CF₃I) to the composition, adding water to the composition, or adding both trifluoroiodomethane (CF₃I) and water to the composition.

Following the separation, the composition may be altered in its characteristics such that the water may be removed from the composition and the CF₃I may be further purified. Suitable methods to purify the CF₃I may include the methods described above, such as distillation, liquid-liquid extraction, or exposure to a drying agent, as well as the method described below.

The product stream from acid absorption and drying comprising CF₃I may be condensed and fed to a first distillation column to provide a first overhead product stream and a first bottoms product stream. The first overhead product stream may comprise impurities with lower boiling points than that of CF₃I as well as the heterogeneous azeotrope or azeotrope-like composition comprising CF₃I and water described above. The low-boiling impurities may be sent to a thermal oxidizer, while the azeotrope or azeotrope-like composition may be phase separated and the water decanted, while the wet CF₃I may be recycled. The first bottoms product stream may be conveyed to a second distillation column to provide a second overhead product stream and a second bottoms product stream. The second overhead product stream may comprise purified CF₃I, while the second bottoms product stream may comprise high-boiling compounds which may be disposed of.

An example of a purification method, as well as the synthesis of CF3I from TFAI discussed in the previous section, is shown in FIG. 3. In this method, a feed stream comprising TFAI (stream 138 from FIG. 2 or stream 126A from FIG. 2A) may be conveyed to a reactor 200 to provide a product stream 202, comprising trifluoroiodomethane, carbon monoxide (CO), TFAI, iodine, R23 (CF₃H), and other impurities, such as trifluoroacetyl fluoride (TFAF), carbon dioxide (CO₂), R13 (CClF₃), TFA, pentafluoropropanone, 133a (2-chloro-1,1,1-trifluoroethane), and pentafluoroiodoethane (C₂F5I), for example. The product stream 202 may be combined with a stream of toluene prior to being conveyed to a first distillation column 204 to provide a first bottoms product stream 206 comprising unreacted TFAI, iodine, and toluene, and a first overhead product stream 208 comprising CF₃I, CO, R23, and other impurities. The first bottoms product stream 206 may be recycled to the second step of the integrated process, described above. The first overhead product stream 208 may be combined with HCl (stream 118 from FIG. 2 or from another source) prior to being conveyed to a second distillation column 210 to provide a second overhead product stream 212 comprising HCl, CO, and other impurities, and a second bottoms product stream 224 comprising trifluoroiodomethane, low-boiling impurities, high-boiling impurities, and residual acid. Alternatively, HCl may be fed into the second distillation column separately, without combining with stream 208. The second overhead product stream 212 may be conveyed to an absorbent bed 214 to provide a first purified product stream 216 comprising HCl and CO. The first purified product stream 216 may be conveyed to an HCl absorber 218 to contact stream 216 with water or a weak HCl solution to provide a stream 220 comprising CO and a second purified product stream 222 comprising an aqueous HCl solution. The stream 220 comprising purified CO may be recovered for use as feedstock for other products including hydrogen via water-gas shift reaction of CO with water or conveyed to a thermal oxidizer. The second purified product stream 222 comprising an aqueous HCl solution may be conveyed to an HCl storage area and sold for profit.

The bottoms product 224 from the second distillation column 210 may be conveyed to a scrubber 226 to remove residual acid, residual TFAC, residual TFAF, and provide stream 228 comprising trifluoroiodomethane, low-boiling impurities, high-boiling impurities, and water. Stream 228 may be conveyed to a dryer 230 to remove water and provide a product stream 232 comprising trifluoroiodomethane, low-boiling impurities, and high-boiling impurities. Stream 232 may be conveyed to a third distillation column 234 to provide a third overhead product stream 236 comprising low-boiling impurities and a third bottoms product stream 238 comprising trifluoroiodomethane and high-boiling impurities. The third overhead product stream 236 may be conveyed to a thermal oxidizer. The third bottoms product stream 238 may be conveyed to a fourth distillation column 240 to provide a fourth overhead product stream 242 comprising purified trifluoromethane and a fourth bottoms product stream 244 comprising high-boiling impurities. The fourth bottoms product stream 244 may be conveyed to a thermal oxidizer. The fourth overhead product stream 242 comprising the purified CF₃I may be conveyed to a storage area.

Independent of the method used to purify the CF₃I, the CF₃I may be or high purity and include only small amounts of impurities, such as TFAC, chlorotrifluoroethane, hexafluoroethane, trifluoromethane, carbon monoxide, HCl, trifluoroacetyl fluoride, hexafluoropropanone, and trifluoroacetaldehyde for example.

TFAC may be present in the CF₃I in an amount of about from 1 ppm (part per million by weight) or of greater, about 10 ppm or greater, about 50 ppm or greater, about 100 ppm or greater, about 150 ppm or greater, about 200 ppm or less, about 250 ppm or less, about 300 ppm or less, about 350 ppm or less, about 400 ppm or less, about 450 ppm or less, about 500 ppm or less, or any value encompassed by these endpoints as determined by gas chromatography (GC).

Chlorotrifluoroethane may be present in the CF₃I in an amount of 1 ppm or of greater, about 10 ppm or greater, about 50 ppm or greater, about 100 ppm or greater, about 150 ppm or greater, about 200 ppm or less, about 250 ppm or less, about 300 ppm or less, about 350 ppm or less, about 400 ppm or less, about 450 ppm or less, about 500 ppm or less, or any value encompassed by these endpoints as determined by gas chromatography (GC).

Hexafluoroethane may be present in the CF₃I in an amount of 1 ppm (part per million by weight) or of greater, about 10 ppm or greater, about 50 ppm or greater, about 100 ppm or greater, about 150 ppm or greater, about 200 ppm or less, about 250 ppm or less, about 300 ppm or less, about 350 ppm or less, about 400 ppm or less, about 450 ppm or less, about 500 ppm or less, or any value encompassed by these endpoints as determined by gas chromatography (GC).

Trifluoromethane may be present in the CF₃I in an amount of 1 ppm or of greater, about 10 ppm or greater, about 50 ppm or greater, about 100 ppm or greater, about 150 ppm or greater, about 200 ppm or less, about 250 ppm or less, about 300 ppm or less, about 350 ppm or less, about 400 ppm or less, about 450 ppm or less, about 500 ppm or less, or any value encompassed by these endpoints as determined by gas chromatography (GC).

Carbon monoxide may be present in the CF₃I in an amount of about 1 ppm or greater, 5 ppm or greater, about 10 ppm or greater, about 20 ppm or greater, about 30 ppm or greater, about 40 ppm or greater, about 50 ppm or less, about 60 ppm or less, about 70 ppm or less, about 80 ppm or less, about 90 ppm or less, about 100 ppm or less, or less, or any value encompassed by these endpoints as determined by thermal conductivity detection (TCD).

HCl may be present in the CF₃I in an amount of about 1 ppm or less, 500 ppb or less, 250 ppb or less, 100 ppb or less, or 50 ppb or less as determined by titration.

Trifluoroacetyl fluoride (TFAF) may be present in the CF₃I in an amount of about 1 ppm or greater, about 10 ppm or greater, about 20 ppm or greater, about 50 ppm or greater, about 75 ppm or greater, about 100 ppm or greater, about 125 ppm or less, about 150 ppm or less, about 175 ppm or less, about 200 ppm or less, about 225 ppm or less, about 250 ppm or less, or any value encompassed by these endpoints as determined by gas chromatography (GC).

Hexafluoropropanone may be present in the CF₃I in an amount of about 1 ppm or greater, about 10 ppm or greater, about 20 ppm or greater, about 50 ppm or greater, about 75 ppm or greater, about 100 ppm or greater, about 125 ppm or less, about 150 ppm or less, about 175 ppm or less, about 200 ppm or less, about 225 ppm or less, about 250 ppm or less, or any value encompassed by these endpoints as determined by gas chromatography (GC).

Trifluoroacetaldehyde may be present in the CF₃I in an amount of about 1 ppm or greater, about 10 ppm or greater, about 20 ppm or greater, about 50 ppm or greater, about 75 ppm or greater, about 100 ppm or greater, about 125 ppm or less, about 150 ppm or less, about 175 ppm or less, about 200 ppm or less, about 225 ppm or less, about 250 ppm or less, or any value encompassed by these endpoints as determined by gas chromatography (GC).

While this invention has been described as relative to exemplary designs, the present invention may be further modified within the spirit and scope of this disclosure. Further, this application is intended to cover such departures from the present disclosure as come within known or customary practice in the art to which this invention pertains.

As used herein, the phrase “within any range defined between any two of the foregoing values” literally means that any range may be selected from any two of the values listed prior to such phrase regardless of whether the values are in the lower part of the listing or in the higher part of the listing. For example, a pair of values may be selected from two lower values, two higher values, or a lower value and a higher value.

### EXAMPLES

### Example 1a: Conversion of H₂ and I₂ to HI

This example illustrates Step 1 of the process disclosed above to produce HI from H₂ and I₂. A continuous vapor phase reaction system utilizes the feed rates shown in Table 1 below to attain an average H₂:I₂ mole ratio of 5.88. The average contact time was 7.9 seconds. Target temperature and pressure in the reactor was 350° C. and 100 psig. The experiment was carried out for 948.5 hours under these conditions. The average I₂ conversion was 97.3% as determined by calculation of mass balance.

Table 1 shows the reaction conditions, mole ratio of H₂ to I₂, and conversion for the experiments performed using 18.5 ml of 20 wt. nickel on alumina catalyst.

TABLE 1

| | | T of I₂ | | | | | I₂ conv., |
|---|---|---|---|---|---|---|---|
| Reactor | | vapor- | H₂ | I₂ | H₂/I₂ | | % (based |
| temp. | Reactor | izer, | flow, | rate, | | Contact | on I₂ |
| ° C. | P, psig | ° C. | ml/min | g/h | (v/v) | Time, s | consumed) |
| 350 | 99.6 | 190 | 440 | 38.2 | 7.3 | 8.1 | 98.6 |
| 350 | 99.9 | 192 | 440 | 39.8 | 7.0 | 8.1 | 98.7 |
| 350 | 99.9 | 194 | 440 | 43.4 | 6.4 | 8.0 | 98.7 |
| 350 | 97.2 | 196 | 440 | 46.3 | 6.0 | 7.9 | 98.5 |
| 350 | 100.3 | 196 | 440 | 49.4 | 5.6 | 7.9 | 98.6 |
| 350 | 99.8 | 195 | 440 | 47.3 | 5.9 | 7.9 | 98.6 |
| 350 | 99.7 | 195 | 440 | 42.3 | 6.6 | 8.0 | 98.5 |
| 350 | 99.7 | 195 | 440 | 42.3 | 6.6 | 8.0 | 98.5 |
| 350 | 99.7 | 196 | 440 | 47.4 | 5.9 | 7.9 | 98.2 |
| 350 | 101.5 | 196 | 440 | 48.7 | 5.7 | 7.9 | 97.2 |
| 350 | 100.9 | 196 | 440 | 52.1 | 5.3 | 7.8 | 97.3 |
| 350 | 100.4 | 196 | 440 | 41.8 | 6.7 | 8.0 | 95.2 |
| 350 | 100.3 | 196 | 440 | 44.5 | 6.3 | 8.0 | 98.3 |
| 350 | 100 | 196 | 440 | 45.8 | 6.1 | 8.0 | 98.2 |
| 350 | 99.9 | 195 | 440 | 47.4 | 5.9 | 7.9 | 97.89 |
| 350 | 99.4 | 196 | 440 | 50.9 | 5.5 | 7.8 | 97.25 |
| 350 | 97.6 | 196 | 440 | 50.6 | 5.5 | 7.8 | 97.37 |
| 350 | 100.1 | 196 | 440 | 53.0 | 5.3 | 7.8 | 96.7 |
| 350 | 100.3 | 195 | 440 | 54.7 | 5.1 | 7.7 | 96.1 |
| 350 | 100.5 | 194 | 440 | 52.2 | 5.3 | 7.8 | 96.5 |
| 350 | 100.2 | 193 | 440 | 52.3 | 5.3 | 7.8 | 96.5 |
| 350 | 99.8 | 192 | 440 | 49.4 | 5.6 | 7.9 | 95.9 |
| 350 | 100.4 | 191 | 440 | 55.7 | 5.0 | 7.7 | 95.7 |
| 350 | 100 | 190 | 440 | 48.4 | 5.8 | 7.9 | 96.6 |
| 350 | 100.1 | 189 | 440 | 47.4 | 5.9 | 7.9 | 96.78 |
| 350 | 99.7 | 188 | 440 | 46.0 | 6.1 | 8.0 | 96.55 |
| 350 | 100.7 | 188 | 440 | 48.4 | 5.8 | 7.9 | 95.99 |
| 350 | 100 | 188 | 440 | 51.3 | 5.4 | 7.8 | 95.5 |

### Example 1b: In-Situ Formation of NiI₂/Al₂O₃

In this Example, a Ni/Al₂O₃ catalyst is converted to a NiI₂/Al₂O₃ catalyst in situ during the HI synthesis reaction.

The 20 wt. % Ni/Al₂O₃ catalyst was activated in pure hydrogen before use, to remove an air passivated layer thereby exposing the active nickel phase. More specifically, 100 mL of catalyst were charged to the reactor and purged with nitrogen gas (400 mL/min), at room temperature, for about 30 minutes. Nitrogen gas flow was discontinued, and hydrogen gas flow (250 mL/min) started. The catalyst was heated to 120° C., at ramp rate of 3° C./min, and held for 1 h. After the hold, the temperature was ramped (3° C./min) to 230° C. and held for an additional hour. The temperature was then ramped (3° C./min) to predetermined reaction temperature.

Except otherwise stated, all materials were used as obtained without further purification. A predetermined amount of iodine was charged into the vaporizer, evacuated, pulse-purged thrice with nitrogen gas, and heated to a predetermined temperature. Hydrogen gas, at a predetermined flow rate was bubbled through the vaporizer. The effluent stream from the vaporizer was contacted with the Ni/Al₂O₃ catalyst inside the reactor. The effluent stream from the reactor was passed through two consecutive iodine collectors, then two successive product collection cylinders (PCC), followed by a water bubbler and finally through a caustic scrubber (10 wt. % KOH/H₂O). The iodine collectors were maintained at about 20° C. (by circulating city water though a copper coil wrapped on the body of the collectors) to assure that unreacted iodine in the reactor effluent stream condensed in the collectors. The anhydrous HI in the reactor effluent stream condensed in the PCC, cooled by liquid nitrogen or acetone-dry ice cooling bath. The uncaptured HI from the PCC was captured in the water bubbler as aqueous HI. The effluent stream from the water bubbler, which was predominantly unreacted hydrogen and entrained aqueous HI, was passed through the caustic scrubber, before it was vented.

The weight of the catalyst was found to increase with time on stream. The change in weight was highest during the first 300 h on stream during which the weight of catalyst increased by about 82.3%. After 600 h, the weight of catalyst increased by about 86.9%, indicating all metallic nickel on the surface of the catalyst had been converted into NiI₂. This observation was corroborated by equilibrium calculations which revealed that the equilibrium concentration of metallic nickel was infinitesimal.

The change in weight of catalyst is due to the formation of nickel (II) iodide (NiI₂), as shown by in Equation 4.

Ni+I₂→NiI₂ Eq. 4

The formation of NiI₂ from metallic nickel and iodine vapors is exothermic and the standard enthalpy of reaction and standard Gibbs Free Energy are −158.8 kJ/mol and −113.8 kJ/mol, respectively. The equilibrium constant at standard conditions is 8.9×10¹⁹. The large equilibrium constant and negative Gibbs Free Energy indicate that the reaction is spontaneous and proceeds readily in the forward direction. This is made possible by the fact that nickel is relatively electropositive compared to other late series metals and can easily loose electron density to form Ni(II) species.

### Example 2a: Recycling of H₂ and I₂

The effluent of the reactor in Example 1 may be compressed to about 200 psig and fed to a distillation column operating at about 190 psig to recover a first recycle stream comprising hydrogen (H₂); a product stream comprising HI, substantially free of impurities; and a second recycle stream comprising (I₂). Moisture in the system will tend to concentrate in the iodine recycle stream. In such a system, the process simulation results below were achieved with a distillation column feed moisture content of 122 ppm. The distillation column includes seven theoretical stages and a mass reflux ratio of 3.4. Moisture will be removed from the column bottoms liquid using a desiccant that is compatible with both HI and I₂. Table 2 below shows the process simulation results.

TABLE 2

| | | | | Column | Column |
|---|---|---|---|---|---|
| Stream | | | Liquid HI | Overhead | Bottoms |
| Description | — | Column Feed | Product | Vapor | Liquid |
| Temperature | F. | 364 | 50 | −45 | 124 |
| Pressure | psia | 204.7 | 204.7 | 204.7 | 204.7 |
| I2 | wt % | 4.132% | 0.000% | 0.000% | 10.000% |
| H2 | wt % | 3.315% | 0.000% | 20.222% | 0.000% |
| HI | wt % | 92.408% | 99.998% | 78.989% | 89.887% |
| WATER | wt % | 0.012% | 0.000% | 0.000% | 0.048% |

Other conditions, including different number of stages, different feed stage, different pressure, different reflux ratio and different boil up ratio may also be used.

### Example 2b: Purification of HI

Once HI has been produced from the reaction of hydrogen and iodine, residual impurities may be removed from the HI by passing it through a purification train. The purified HI may be analyzed by ¹H NMR and/or titration. More specifically, the concentration of HI may be determined by titration or ¹H NMR, while the total concentration of iodine species may be determined by titration with thiosulphate. A representative composition is shown in Table 2A below.

TABLE 2A

| Component | Amount (ppm) |
|---|---|
| Iodine species (I₂ and HI₃) | <3000 |
| Non-volatile residue (NVR) | <300 |
| H₂ gas | <100 |
| Other organics | Trace |
| Water | Trace |

The NVR in the table above may include one or more components selected from the group consisting of iodine, diiodopropane, tertbutyl iodide, iodopropane, iodopropene, and other iodo-hydrocarbons.

### Example 3a: Formation of TFAI from TFAC and HI

In the following examples, the manufacture of TFAI from TFAC and HI is demonstrated. A three-quarter inch metal tube located in a temperature control device such as an oven or sand bath was used as a reactor preloaded with certain amount of catalyst or without catalyst. A certain amount of TFAC and HI was co-fed into the heated fixed bed tubular reactor to conduct the reaction. The reactor effluent was passed through an electrically heat-traced line to prevent the condense of TFAI and directed to a cylinder located in a dry-ice Dewar to capture the crude product. The trace amount of vapor escaping from the dry ice trap was directed to a water scrubber and a caustic scrubber. A pressure transducer and a control valve were also installed at the outlet of the reactor to control the reaction pressure.

Periodically, samples were taken from the reactor effluent, and the composition of the organic compounds in the samples were measured by gas chromatography (GC). Graph areas provided by the GC analysis for each of the organic compounds were combined to provide a GC area percentage (GC area %) of the total organic compounds. The contact time in the reactor was calculated based on the combined feed rates of the hydrogen iodide and the TFAC.

At the end of the run time of the reaction, the system was shut down and weight changes from all containers were checked for mass balance purpose. The liquid crude product collected in the dry-ice trap was sampled and analyzed by GC and/or GCMS.

### Example 3b: TFAC Conversion with 20 mL of Catalyst

Table 3 below shows the conversion of TFAC (“Conv. %”) for 28 different runs. In runs 1-26, a ¾″ Inconel 600 reactor was used, while in runs 27 and 28, a ¾″ Inconel 625 reactor was used. In those cases in which a catalyst was present, 20 mL of catalyst was used. Among the catalysts tested were 5% palladium on alumina, silica carbide catalysts (SiC2-E3-THP and SiC1-E3-P), activated carbon catalysts (Norit ROX0.8, CPG CF12X40, OLC12X30 and JEChem C2X8/12), and Inconel 625 wire mesh (designated as “wire mesh” in the table below). Temperatures ranging from 40° C. to 210° C. were tested, as were pressures ranging from 0 psig (ambient, designated as “Amb.”) to 20 psig.

TABLE 3

| | | | | | TFA | | TFAC: | | |
|---|---|---|---|---|---|---|---|---|---|
| | | | | Run | C | HI | HI | Con- | |
| | | T | P | time | feed | feed | mole | tact | Conv. |
| Run | Cat. | ° C. | psig | h | g/h | g/h | ratio | times | % |
| 1 | — | 210 | Amb | 30 | 16.2 | 15.8 | 0.99:1 | 21 | 43.46 |
| 2 | 0.5% Pd/ | 210 | Amb | 8 | 23.1 | 15.6 | 1.43:1 | 6.1 | 78.01 |
| | Al₂O₃ | | | | | | | | |
| 3 | SiC₂-E3- | 210 | Amb | 26 | 18.4 | 15.9 | 1.12:1 | 6.8 | 81.90 |
| | HP | | | | | | | | |
| 4 | SiC₂-E3- | 210 | Amb | 48.5 | 18.5 | 18.7 | 0.95:1 | 6.3 | 81.38 |
| | HP | | | | | | | | |
| 5 | SiC₂-E3- | 180 | Amb | 12 | 16.1 | 15.7 | 0.99:1 | 7.8 | 82.77 |
| | HP | | | | | | | | |
| 6 | SiC₂-E3- | 150 | Amb | 10 | 16.1 | 15.7 | 0.99:1 | 8.4 | 82.49 |
| | HP | | | | | | | | |
| 7 | SiC₂-E3- | 120 | Amb | 8 | 16.1 | 15.7 | 0.99:1 | 9 | 83.37 |
| | HP | | | | | | | | |
| 8 | SiC₂-E3- | 90 | Amb | 12 | 18.1 | 18.2 | 0.96:1 | 8.6 | 88.30 |
| | HP | | | | | | | | |
| 9 | SiC₂-E3- | 60 | Amb | 12 | 18.1 | 18.2 | 0.96:1 | 9.4 | 87.59 |
| | HP | | | | | | | | |
| 10 | SiC₂-E3- | 40 | Amb | 16.5 | 17.9 | 17.4 | 0.99:1 | 10.2 | 87.12 |
| | HP | | | | | | | | |
| 11 | SiC₂-E3- | 60 | 5 | 13 | 17.9 | 17.4 | 0.99:1 | 12.9 | 85.89 |
| | HP | | | | | | | | |
| 12 | SiC₂-E3- | 60 | 15 | 13 | 17.9 | 17.4 | 0.99:1 | 19.4 | 85.69 |
| | HP | | | | | | | | |
| 13 | Norit | 60 | 15 | 39 | 18.1 | 18.9 | 0.92:1 | 18.5 | 85.34 |
| | ROX0.8 | | | | | | | | |
| 14 | Norit | 90 | 15 | 12 | 18.1 | 18.9 | 0.92:1 | 17.0 | 87.38 |
| | ROX0.8 | | | | | | | | |
| 15 | Norit | 120 | 15 | 12.25 | 18.1 | 18.9 | 0.92:1 | 15.7 | 85.22 |
| | ROX0.8 | | | | | | | | |
| 16 | Norit | 40 | 15 | 15.25 | 18.1 | 18.9 | 0.92:1 | 19.7 | 79.08 |
| | ROX0.8 | | | | | | | | |
| 17 | CPG | 60 | 15 | 24.5 | 17.1 | 20.8 | 0.79:1 | 18.0 | 90.82 |
| | CF12X40 | | | | | | | | |
| 18 | CPG | 90 | 15 | 20 | 17.1 | 20.8 | 0.79 | 16.5 | 89.46 |
| | CF12X40 | | | | | | | | |
| 19 | OLC12X | 60 | 15 | 17.5 | 18.8 | 22.7 | 0.80:1 | 16.5 | 90.28 |
| | 30 | | | | | | | | |
| 20 | OLC12X | 60 | 15 | 13 | 18.8 | 22.7 | 0.80:1 | 15.1 | 88.70 |
| | 30 | | | | | | | | |
| 21 | JEChem | 60 | 15 | 18 | 18.0 | 22.0 | 0.79:1 | 17.1 | 88.86 |
| | C2X8/12 | | | | | | | | |
| 22 | JEChem | 90 | 15 | 12 | 18.0 | 22.0 | 0.79:1 | 15.7 | 89.31 |
| | C2X8/12 | | | | | | | | |
| 23 | Norit | 90 | 15 | 24 | 17.6 | 22.7 | 0.75:1 | 15.6 | 88.43 |
| | ROX0.8 | | | | | | | | |
| 24 | Norit | 90 | 15 | 30 | 18.6 | 12.8 | 1.40:1 | 20.1 | 71.38 |
| | ROX0.8 | | | | | | | | |
| 25 | SiC1-E3- | 90 | 20 | 49 | 15.98 | 11.18 | 1.38:1 | 27.1 | 68.28 |
| | P | | | | | | | | |
| 26 | SiC1-E3- | 90 | 20 | 24 | 17.04 | 12.96 | 1.27:1 | 24.5 | 78.09 |
| | P | | | | | | | | |
| 27 | — | 90 | 20 | 11.5 | 18.26 | 11.30 | 1.56:1 | 71.7 | 37.56 |
| 28 | wire | 90 | 20 | 14.25 | 19.79 | 13.33 | 1.43:1 | 22.3 | 33.54 |
| | mesh | | | | | | | | |

The products of the runs in Table 3 were subjected to GC analysis, the results of which are shown below in Table 4. As can be seen therein, TFAI was formed as major product in all but one of the 28 runs.

TABLE 4

| | GC area % | | | | |
|---|---|---|---|---|---|
| Run | TFAC | CF₃I | R133 isomer | TFAI | Other |
| 1 | 56.22 | 0.002 | 0.037 | 38.90 | 4.81 |
| 2 | 21.45 | 0.43 | 0.024 | 73.79 | 4.31 |
| 3 | 17.89 | 2.91 | 0.012 | 76.41 | 2.78 |
| 4 | 18.03 | 3.22 | 0.017 | 75.73 | 3.00 |
| 5 | 17.11 | 0.25 | 0.022 | 79.99 | 2.63 |
| 6 | 17.44 | 0.012 | 0.027 | 80.71 | 1.81 |
| 7 | 16.56 | N/A | 0.037 | 81.63 | 1.77 |
| 8 | 11.27 | N/A | 0.026 | 83.13 | 5.57 |
| 9 | 12.35 | N/A | 0.027 | 83.70 | 3.92 |
| 10 | 12.81 | N/A | 0.027 | 82.80 | 4.36 |
| 11 | 14.05 | N/A | 0.029 | 83.26 | 2.66 |
| 12 | 14.27 | N/A | 0.030 | 83.51 | 2.19 |
| 13 | 14.59 | N/A | 0.031 | 83.57 | 1.81 |
| 14 | 12.55 | N/A | 0.029 | 85.86 | 1.56 |
| 15 | 14.50 | 0.0014 | 0.032 | 82.36 | 3.11 |
| 16 | 20.85 | N/A | 0.035 | 75.95 | 3.17 |
| 17 | 9.01 | N/A | 0.031 | 87.22 | 3.74 |
| 18 | 10.45 | N/A | 0.033 | 86.09 | 3.43 |
| 19 | 9.60 | N/A | 0.032 | 86.04 | 4.33 |
| 20 | 11.21 | N/A | 0.036 | 85.09 | 3.67 |
| 21 | 11.06 | N/A | 0.035 | 85.31 | 3.60 |
| 22 | 10.62 | N/A | 0.036 | 86.16 | 3.18 |
| 23 | 11.48 | N/A | 0.038 | 86.10 | 2.38 |
| 24 | 28.50 | N/A | 0.040 | 69.93 | 1.53 |
| 25 | 62.36 | N/A | 0.069 | 35.30 | 2.27 |
| 26 | 66.36 | N/A | 0.072 | 30.82 | 2.75 |
| 27 | 31.57 | N/A | 0.047 | 65.66 | 2.83 |
| 28 | 21.72 | N/A | 0.050 | 76.03 | 2.20 |

### Example 3c: Long-Term Test with Silica Carbide Catalyst

A ¾″ Inconel 600 reactor charged with 20 mL of silica carbide catalyst (SiC1-E3-M) was used in a long-term test at a reaction temperature of 90° C. with a reactor outlet pressure of 20 psig. Periodically, the system was shut down to check the mass balance and collect the crude product for analysis, with the reaction conditions and results listed in Table 5. Conversion of TFAC, along with selectivity for TFAI and CF₃I, is given in mole percent.

TABLE 5

| | | | | | | | | | Total time |
|---|---|---|---|---|---|---|---|---|---|
| | Feed | | TFAC/HI, | | Time on | | | | on |
| | rate, g/h | | mol/ | Contact | stream, | TFAC conv. | TFAI sel. | CF₃I sel. | stream, |
| Run | TFAC | HI | mol | time, s | h | % | % | % | h |
| 29 | 16.64 | 14.11 | 1.14:1 | 23.92 | 96.00 | 77.92 | 99.96 | 0.00 | 96.00 |
| 30 | 16.81 | 13.03 | 1.25:1 | 24.67 | 96.00 | 74.99 | 99.95 | 0.00 | 192.00 |
| 31 | 17.35 | 12.71 | 1.32:1 | 24.50 | 96.00 | 73.53 | 99.95 | 0.00 | 288.00 |
| 32 | 16.41 | 12.74 | 1.24:1 | 25.25 | 100.00 | 72.24 | 99.90 | 0.00 | 388.00 |
| 33 | 16.83 | 12.34 | 1.32:1 | 25.24 | 67.25 | 81.75 | 99.91 | 0.00 | 455.25 |

The TFAC conversion was an average based on TFAC GC area %. TFAI and CF3I selectivity were calculated based on gas chromatography/mass spectrometry (GC/MS) analysis of the crude product collected in the dry ice trap after each run. Neither CF₃I formation nor catalyst deactivation was observed during 455 hours of operation.

### Example 3d: Long-Term Test with Activated Carbon Catalyst

A ¾″ Inconel 600 reactor charged with 20 mL of an activated carbon catalyst (NORIT ROX0.8) for a long-term test at a reaction temperature of 90° C. and a reactor outlet pressure ranging from 20 psig to 50 psig. Periodically, the system was shut down to check the mass balance and collect the crude product for analysis, with the reaction conditions and results listed in Table 6. Conversion of TFAC, along with selectivity for TFAI and CF₃I, is given in mole percent.

TABLE 6

| | | | | | | | | | | Total |
|---|---|---|---|---|---|---|---|---|---|---|
| | | | | | | | | | | time on |
| | P, | Feed rate, g/h | | TFAC/HI, | Contact | Time on | TFAC | TFAI | CF3I | stream, |
| Run | psig | TFAC | HI | mol/mol | time, s | stream, h | conv. % | sel. % | sel. % | h |
| 34 | 50 | 24.0 | 12.5 | 1.86 | 37.7 | 97.75 | 71.81 | N/A | N/A | 97.75 |
| 35 | 50 | 11.7 | 12.0 | 0.95 | 57.9 | 87.50 | 94.03 | N/A | N/A | 185.25 |
| 36 | 30 | 19.8 | 14.0 | 1.36 | 28.1 | 42.75 | 78.88 | N/A | N/A | 228.00 |
| 37 | 20 | 19.8 | 14.0 | 1.36 | 21.8 | 52.00 | 56.81 | N/A | N/A | 280.00 |
| 38 | 20 | 16.3 | 9.8 | 1.61 | 28.4 | 12.00 | 55.68 | N/A | N/A | 292.00 |
| 39 | 20 | 18.4 | 12.1 | 1.47 | 24.2 | 93.25 | 70.05 | 99.89 | 0.00 | 385.25 |
| 40 | 30 | 17.8 | 13.1 | 1.32 | 30.7 | 98.50 | 70.49 | 99.95 | 0.00 | 483.75 |
| 41 | 30 | 14.6 | 12.9 | 1.10 | 34.4 | 100.00 | 71.71 | 99.94 | 0.00 | 583.75 |
| 42 | 20 | 18.71 | 12.95 | 1.39 | 23.3 | 97.50 | 73.40 | 99.98 | 0.00 | 681.25 |
| 43 | 20 | 19.25 | 19.66 | 0.95 | 18.9 | 92.50 | 84.02 | 99.74 | 0.00 | 773.75 |
| 44 | 20 | 19.16 | 17.322 | 1.07 | 20.2 | 91.50 | 77.01 | 99.86 | 0.00 | 865.25 |
| 45 | 20 | 19.13 | 11.47 | 1.61 | 24.11 | 86.00 | 60.70 | 99.79 | 0.00 | 951.25 |
| 46 | 20 | 18.98 | 13.72 | 1.34 | 22.53 | 46.00 | 70.84 | 99.76 | 0.00 | 997.25 |
| 47 | 20 | 20.15 | 10.00 | 1.95 | 24.50 | 6.50 | 56.18 | 97.28 | 0.00 | 1003.75 |
| 48 | 20 | 19.03 | 13.09 | 1.40 | 22.94 | 58.00 | 66.98 | 99.69 | 0.00 | 1061.75 |
| 49 | 20 | 19.05 | 11.88 | 1.55 | 23.84 | 48.50 | 63.61 | 99.98 | 0.00 | 1110.25 |
| 50 | 20 | 19.25 | 12.29 | 1.51 | 23.38 | 48.00 | 64.21 | 99.98 | 0.00 | 1158.25 |
| 51 | 20 | 12.73 | 8.85 | 1.39 | 34.14 | 48.00 | 52.92 | 99.85 | 0.00 | 1206.25 |
| 52 | 20 | 18.99 | 13.07 | 1.40 | 22.98 | 25.17 | 64.97 | 99.98 | 0.00 | 1231.42 |
| 53 | 20 | 19.10 | 14.35 | 1.29 | 22.01 | 100 | 73.38 | 99.97 | 0.00 | 1331.42 |
| 54 | 20 | 19.01 | 14.51 | 1.27 | 21.96 | 101.25 | 67.59 | 99.94 | 0.00 | 1432.67 |
| 55 | 20 | 18.70 | 11.96 | 1.51 | 24.05 | 23.00 | 59.30 | 99.97 | 0.00 | 1455.67 |
| 56 | 20 | 18.85 | 12.573 | 1.45 | 23.45 | 96.00 | 60.89 | 99.88 | 0.00 | 1551.67 |
| 57 | 20 | 18.67 | 5.36 | 3.37 | 30.87 | 45.00 | 24.23 | 99.90 | 0.00 | 1596.67 |
| 58 | 20 | 18.91 | 12.94 | 1.41 | 23.14 | 96.00 | 67.87 | 99.96 | 0.00 | 1692.67 |
| 59 | 20 | 18.81 | 12.73 | 1.43 | 23.36 | 96.00 | 68.66 | 99.98 | 0.00 | 1788.67 |
| 60 | 20 | 18.77 | 15.28 | 1.19 | 21.61 | 96.00 | 72.98 | 99.94 | 0.00 | 1884.67 |
| 61 | 20 | 18.92 | 11.39 | 1.60 | 24.34 | 100.00 | 60.64 | 99.93 | 0.00 | 1984.67 |
| 62 | 20 | 18.81 | 13.22 | 1.37 | 23.00 | 67.25 | 59.38 | 99.96 | 0.00 | 2051.92 |

The TFAC conversion was an average based on TFAC GC area %. TFAI and CF3I selectivity were calculated based on gas chromatography/mass spectrometry (GC/MS) analysis of the crude product collected in the dry ice trap after each run. Neither CF₃I formation nor catalyst deactivation was observed during 2052 hours of operation.

### Example 3e: TFAC Conversion with 74 mL of Catalyst

Table 7 below shows the conversion of TFAC (“Cony. % o”) for four different runs. In each run, a ¾″ Inconel 625 reactor charged with 74 ml of an activated carbon catalyst (Norit ROX0.8) was used. The reactor was preheated to 90° C. with the reactor outlet pressure controlled at 70 psig. In no case was CF₃I formation observed.

TABLE 7

| | Run | TFAC | HI | TFAC:HI | | |
|---|---|---|---|---|---|---|
| | time | feed | feed | mole/ | Contact | Conv. |
| Run | h | g/h | g/h | mole | times | % |
| 63 | 92.5 | 124.2 | 81.9 | 1.46:1 | 32.7 | 69.1 |
| 64 | 71.5 | 71.5 | 107.9 | 1.26:1 | 35.2 | 80.18 |
| 65 | 168.2 | 36.6 | 78.8 | 0.45:1 | 57.7 | 96.6 |
| 66 | 48.3 | 16.1 | 136.8 | 0.11:1 | 43.3 | 99.4 |

### Example 3f: TFAI Reactor Feed Composition

A representative composition of the reactor feed to reaction TFAC+HI Q TFAI+HI is shown in Table 7A below.

TABLE 7A

| | Amount | | Desired amount | |
|---|---|---|---|---|
| Component | by weight | | by weight | |
| Sum of TFAC and HI | 99.4% (58.8% TFAC/40.6% HI) | | >99% | |
| SO2 | 147 | ppm | <250 | ppm |
| Sum of I2 and HI3 | 1201 | ppm | <2000 | ppm |
| Iodohydrocarbons | 358 | ppm | <500 | ppm |
| H₂ gas | 159 | ppm | <500 | ppm |
| CF3I | 2464 | ppm | <5000 | ppm |

| | | |
|---|---|---|
| TFAI | Trace | Trace |
| HCl | Trace | Trace |
| Others | Balance | Balance |

The reactor feed material may be analyzed by GC, GCMS, ¹H NMR and/or titration.

### Example 4: Formation and Separation of TFAI in an Integrated Bench Scale Unit

This example illustrates a continuous process to produce TFAI from TFAC and HI as well as the separation of TFAI from reaction products. The integrated bench scale unit consisted of a TFAC and HI feed system, a reactor system, a separation system with two distillation columns to remove HCl from the reaction crude product in the first column and to remove unreacted TFAC and HI from TFAI in the second column, and a KOH scrubber system. The reactor system consisted of a preheater and a reactor, with the reactor loaded with catalyst. Both preheater and reactor were placed in a sand-bath. Both distillation columns had the same setup and consisted of a 10 gallon reboiler, 2″ ID×120″ long column with Goodloe 2″ dia×6″ thick structured metal packing) and a tube in shell condenser.

During the operation, the reactor was loaded with 74 ml of NORIT ROX0.8 activated carbon as the catalyst. The reactor system was preheated to 90° C. with nitrogen purge. After the reactor temperature reached 90° C., 120 grams/h of liquid TFAC and 82 grams/h of vapor HI were fed into the preheater and the vaporized feed mixture was fed into the reactor for continuous reaction. The reaction pressure was controlled at 70 psig by a reactor outlet control valve at the startup, and the crude product was then fed into the first distillation column to remove HCl from the crude product. The first distillation column reboiler was heated by saturated steam at 30 psig and city water mixture to 55-60° C., and the condenser was cooled by liquid nitrogen. HCl was vented off from the column overhead to the KOH scrubber system periodically to maintain the column overhead pressure at 70 psig. After the first column pressure reached 70 psig, the reactor outlet pressure control valve was by-passed to have the reaction pressure controlled by the first column overhead pressure. The first column reboiler material containing mainly TFAI, and unreacted TFAC and HI was fed into the second distillation column with the column reboiler temperature controlled at 55-60° C. by saturated steam at 30 psig, and the overhead pressure controlled at 28 psig. The overhead stream containing mainly TFAC and HI was collected into a cylinder for recycle, and the reboiler material contained mainly TFAI with some unreacted TFAC and HI was collected into a cylinder for further purification.

During 985 hours of operation, the typical reactor effluent stream contained 30.7% TFAC and 69.1% TFAI, balanced with other impurities, representing a TFAC conversion of 69.2% and a selectivity for TFAI of 99.8%. By ion chromatography (IC) analysis, the first column overhead stream contained only HCl without any other compounds. By gas chromatography (GC) analysis, the first column reboiler contained 23.2% TFAC, 76.1% TFAI, balanced with others, the second column overhead stream contained 98.7% TFAC, 0.8% TFAI, balanced with others, and the second column reboiler contained 3.5% TFAC, 95.4% TFAI, balanced with others.

### Example 5: Conversion of TFAI to CF₃I

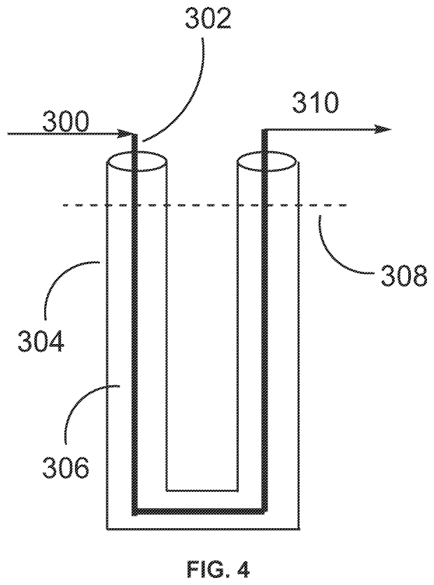

A combination trifluoroacetyl iodide (TFAI) vaporizer/superheater/pyrolysis reactor was immersed in a constant temperature sand bath. As shown in FIG. 4, the inlet 300 led to a U-shaped ¾″ OD Incoloy 825 tube 302 into which was inserted a 0.495-inch outer diameter Inconel 600 electric heating element (EHE) 304, which left a small annular space between the EHE and the inside wall of the Incoloy tube 306. The reaction set up was placed in a sand bath to level 308 in FIG. 4, and the sand bath was heated to 200° C. As the TFAI passed through the annular space, the pyrolysis of TFAI to provide CF₃I and CO occurred, and the product exited through the outlet 310.

Experiments were performed using four reaction conditions to determine the effect of temperature and contact time on both TFAI conversion and CF₃I selectivity. The purity of TFAI that was used in the experiments was 99.33 by GC area %.

Condition #1 was defined as a TFAI feed rate of 0.25 lb/hr, a pressure of 25 psig, a heater element temperature in the range of 390±1° C., and a contact time through the annular space of 2.1 seconds. The average TFAI conversion was 68.6 mole %, and the CF₃I selectivity was 99.5 mole %. Both the conversion and selectivity were relatively steady throughout the experiment. A table of the reactor exit GC analysis during the experiment can be found in Table 8 below, shown as % area. TFAF is trifluoroacetyl fluoride, PFP is pentafluoropropanone, 133a is 2-chloro-1,1,1-trifluoroethane, and TFA is trifluoroacetic acid. No iodomethane (CH₃I) formation was observed. On-stream time is shown in hours in the first column.

TABLE 8

| Hours | TFAF | CF₃H | TFAC | PFP | CF₃I | 133a | C₂F₅I | TFAI | TFA | Other |
|---|---|---|---|---|---|---|---|---|---|---|
| 12 | 0.03 | 0.10 | 0.16 | 0.002 | 63.10 | 0.01 | 0.00 | 35.92 | 0.44 | 0.24 |
| 20 | 0.02 | 0.09 | 0.08 | 0.000 | 56.84 | 0.01 | 0.00 | 42.02 | 0.73 | 0.21 |
| 28 | 0.04 | 0.11 | 0.15 | 0.000 | 63.05 | 0.01 | 0.00 | 35.93 | 0.30 | 0.41 |
| 36 | 0.04 | 0.11 | 0.15 | 0.000 | 61.94 | 0.01 | 0.00 | 36.99 | 0.36 | 0.40 |
| 44 | 0.02 | 0.09 | 0.14 | 0.000 | 61.59 | 0.01 | 0.00 | 37.58 | 0.35 | 0.22 |
| 52 | 0.06 | 0.10 | 0.14 | 0.003 | 61.70 | 0.01 | 0.00 | 37.39 | 0.38 | 0.20 |
| 60 | 0.05 | 0.11 | 0.11 | 0.008 | 65.46 | 0.01 | 0.00 | 33.12 | 0.92 | 0.21 |
| 68 | 0.02 | 0.11 | 0.16 | 0.000 | 65.69 | 0.01 | 0.00 | 33.44 | 0.33 | 0.25 |
| 72 | 0.02 | 0.10 | 0.12 | 0.005 | 66.07 | 0.01 | 0.00 | 32.92 | 0.63 | 0.11 |
| 80 | 0.02 | 0.11 | 0.16 | 0.008 | 61.17 | 0.01 | 0.01 | 38.11 | 0.23 | 0.17 |
| 92 | 0.02 | 0.11 | 0.16 | 0.000 | 61.73 | 0.01 | 0.00 | 37.54 | 0.21 | 0.22 |
| 100 | 0.02 | 0.12 | 0.15 | 0.006 | 65.55 | 0.01 | 0.00 | 33.33 | 0.52 | 0.29 |
| 120 | 0.02 | 0.10 | 0.15 | 0.003 | 61.61 | 0.01 | 0.00 | 37.66 | 0.24 | 0.20 |
| 132 | 0.02 | 0.10 | 0.15 | 0.002 | 60.65 | 0.01 | 0.00 | 38.62 | 0.29 | 0.15 |
| 136 | 0.03 | 0.12 | 0.13 | 0.000 | 76.55 | 0.01 | 0.00 | 22.76 | 0.25 | 0.16 |
| 140 | 0.03 | 0.12 | 0.14 | 0.000 | 79.23 | 0.01 | 0.00 | 19.73 | 0.25 | 0.48 |
| 144 | 0.03 | 0.11 | 0.14 | 0.000 | 66.89 | 0.01 | 0.00 | 32.48 | 0.19 | 0.14 |
| 148 | 0.03 | 0.11 | 0.13 | 0.000 | 74.41 | 0.01 | 0.00 | 24.87 | 0.31 | 0.15 |
| 156 | 0.02 | 0.10 | 0.15 | 0.000 | 57.22 | 0.01 | 0.00 | 42.10 | 0.24 | 0.16 |
| 160 | 0.03 | 0.10 | 0.13 | 0.000 | 60.78 | 0.01 | 0.00 | 38.54 | 0.28 | 0.13 |
| 164 | 0.03 | 0.10 | 0.15 | 0.001 | 57.19 | 0.01 | 0.00 | 42.04 | 0.23 | 0.25 |
| 168 | 0.02 | 0.09 | 0.12 | 0.000 | 57.12 | 0.01 | 0.00 | 42.13 | 0.34 | 0.16 |
| 172 | 0.06 | 0.11 | 0.12 | 0.000 | 60.84 | 0.01 | 0.01 | 38.32 | 0.33 | 0.20 |
| 188 | 0.04 | 0.11 | 0.13 | 0.000 | 76.15 | 0.01 | 0.01 | 23.09 | 0.35 | 0.11 |
| 192 | 0.01 | 0.06 | 0.14 | 0.000 | 69.96 | 0.00 | 0.00 | 28.36 | 1.33 | 0.13 |
| 196 | 0.02 | 0.10 | 0.13 | 0.000 | 73.20 | 0.01 | 0.00 | 25.99 | 0.40 | 0.14 |
| 200 | 0.02 | 0.10 | 0.13 | 0.000 | 67.92 | 0.01 | 0.00 | 31.21 | 0.45 | 0.16 |
| 204 | 0.02 | 0.12 | 0.12 | 0.000 | 74.18 | 0.00 | 0.00 | 25.07 | 0.39 | 0.08 |
| 208 | 0.02 | 0.10 | 0.12 | 0.000 | 61.84 | 0.00 | 0.00 | 37.38 | 0.39 | 0.14 |
| 212 | 0.02 | 0.10 | 0.09 | 0.000 | 66.94 | 0.01 | 0.00 | 32.21 | 0.46 | 0.18 |
| 216 | 0.05 | 0.11 | 0.11 | 0.008 | 65.46 | 0.01 | 0.00 | 33.12 | 0.92 | 0.21 |
| 220 | 0.03 | 0.10 | 0.13 | 0.000 | 60.78 | 0.01 | 0.00 | 38.54 | 0.28 | 0.13 |
| 224 | 0.04 | 0.11 | 0.15 | 0.000 | 63.05 | 0.01 | 0.00 | 35.93 | 0.30 | 0.41 |
| 228 | 0.02 | 0.12 | 0.15 | 0.006 | 65.55 | 0.01 | 0.00 | 33.33 | 0.52 | 0.29 |
| 232 | 0.02 | 0.10 | 0.13 | 0.000 | 67.92 | 0.01 | 0.00 | 31.21 | 0.45 | 0.16 |
| 236 | 0.03 | 0.11 | 0.14 | 0.000 | 66.89 | 0.01 | 0.00 | 32.48 | 0.19 | 0.14 |
| 240 | 0.04 | 0.11 | 0.15 | 0.000 | 61.94 | 0.01 | 0.00 | 36.99 | 0.36 | 0.40 |
| 244 | 0.02 | 0.10 | 0.09 | 0.000 | 66.94 | 0.01 | 0.00 | 32.21 | 0.46 | 0.18 |
| Avg. | 0.29 | 0.105 | 0.135 | 0.001 | 65.135 | 0.007 | 0.004 | 33.964 | 0.411 | 0.210 |

Condition #2 was defined as a TFAI feed rate of 0.08 lb/hr, a pressure of 25 psig, a heater element temperature in the range of 332.5±2.5° C., and a contact time through the annular space of 7.2 seconds. The average TFAI conversion was 68.1 mole 00 and CF₃I selectivity was 99.5 mole 00. The study ran for 52 hours.

A table of the reactor exit GC analysis during the experiment can be found in Table 9 below, shown as 00 area. TFAF is trifluoroacetyl fluoride, 133a is 2-chloro-1,1,1-trifluoroethane, and TFA is trifluoroacetic acid. Pentafluoropropane, pentafluoroiodoethane (C₂F5I), and iodomethane (CH₃I) formation were not observed. On-stream time is shown in hours in the first column.

TABLE 9

| Hours | TFAF | CF₃H | TFAC | CF₃I | 133a | TFAI | TFA | Other |
|---|---|---|---|---|---|---|---|---|
| 8 | 0.02 | 0.13 | 0.14 | 67.37 | 0.00 | 31.85 | 0.34 | 0.14 |
| 12 | 0.02 | 0.13 | 0.13 | 64.67 | 0.00 | 34.55 | 0.35 | 0.16 |
| 16 | 0.01 | 0.11 | 0.13 | 61.49 | 0.01 | 37.72 | 0.39 | 0.15 |
| 20 | 0.01 | 0.11 | 0.12 | 63.26 | 0.01 | 36.00 | 0.35 | 0.15 |
| 24 | 0.01 | 0.11 | 0.12 | 63.26 | 0.01 | 36.00 | 0.35 | 0.15 |
| 28 | 0.01 | 0.11 | 0.12 | 67.82 | 0.01 | 31.41 | 0.38 | 0.15 |
| 32 | 0.01 | 0.13 | 0.13 | 65.17 | 0.01 | 33.99 | 0.40 | 0.15 |
| 36 | 0.01 | 0.11 | 0.12 | 60.29 | 0.00 | 38.87 | 0.45 | 0.15 |
| 40 | 0.01 | 0.11 | 0.12 | 68.16 | 0.00 | 30.95 | 0.49 | 0.16 |
| 44 | 0.01 | 0.11 | 0.09 | 63.81 | 0.01 | 35.37 | 0.43 | 0.17 |
| Avg. | 0.011 | 0.116 | 0.123 | 64.529 | 0.005 | 34.671 | 0.394 | 0.151 |

Condition #3 was defined as a TFAI feed rate of 0.15 lb/hr, a pressure of 25 psig, a heater element temperature in the range of 390±1° C., and a contact time through the annular space of 3.5 seconds. The average TFAI conversion was 85.9 mole % and CF₃I selectivity was 99.5 mole %. The study ran for 44 hours.

A table of the reactor exit GC analysis during the experiment can be found in Table 10 below, shown as % area. TFAF is trifluoroacetyl fluoride, PFP is pentafluoropropanone, 133a is 2-chloro-1,1,1-trifluoroethane, and TFA is trifluoroacetic acid. No iodomethane (CH₃I) formation was observed. On-stream time is shown in hours in the first column.

TABLE 10

| Hours | TFAF | CF₃H | TFAC | PFP | CF₃I | 133a | C₂F₅I | TFAI | TFA | Other |
|---|---|---|---|---|---|---|---|---|---|---|
| 8 | 0.05 | 0.13 | 0.16 | 0.002 | 85.41 | 0.01 | 0.01 | 13.81 | 0.27 | 0.16 |
| 12 | 0.04 | 0.13 | 0.12 | 0.000 | 86.25 | 0.01 | 0.01 | 12.82 | 0.38 | 0.25 |
| 16 | 0.04 | 0.12 | 0.14 | 0.000 | 87.94 | 0.01 | 0.01 | 11.18 | 0.31 | 0.25 |
| 20 | 0.04 | 0.12 | 0.14 | 0.000 | 87.94 | 0.01 | 0.01 | 11.18 | 0.31 | 0.25 |
| 24 | 0.04 | 0.14 | 0.06 | 0.000 | 82.58 | 0.01 | 0.01 | 16.60 | 0.43 | 0.13 |
| 28 | 0.05 | 0.14 | 0.08 | 0.000 | 82.14 | 0.01 | 0.01 | 16.97 | 0.42 | 0.19 |
| 32 | 0.04 | 0.12 | 0.13 | 0.000 | 77.81 | 0.01 | 0.01 | 21.42 | 0.35 | 0.11 |
| 36 | 0.05 | 0.12 | 0.14 | 0.000 | 78.52 | 0.01 | 0.01 | 20.63 | 0.31 | 0.22 |
| 40 | 0.05 | 0.12 | 0.12 | 0.000 | 82.91 | 0.01 | 0.01 | 16.27 | 0.37 | 0.14 |
| 44 | 0.05 | 0.14 | 0.08 | 0.000 | 82.14 | 0.01 | 0.01 | 16.60 | 0.43 | 0.13 |
| Avg. | 0.046 | 0.129 | 0.117 | 0.000 | 83.364 | 0.006 | 0.008 | 15.748 | 0.357 | 0.183 |

Condition #4 was defined as a TFAI feed rate of 0.35 lb/hr, a pressure of 25 psig, a heater element temperature in the range of 390±1° C., and a contact time through the annular space of 1.5 seconds. The average TFAI conversion was 59.9 mole % and CF₃I selectivity was 99.5 mole %. The study ran for 44 hours. A table of the reactor exit GC analysis during the experiment can be found in Table 11 below, shown as % area. TFAF is trifluoroacetyl fluoride, PFP is pentafluoropropanone, 133a is 2-chloro-1,1,1-trifluoroethane, and TFA is trifluoroacetic acid. Neither pentafluoroiodoethane (C₂F₅I) nor iodomethane (CH₃I) formation were observed. On-stream time is shown in hours in the first column.

TABLE 11

| Hours | TFAF | CF₃H | TFAC | PFP | CF₃I | 133a | TFAI | TFA | Other |
|---|---|---|---|---|---|---|---|---|---|
| 8 | 0.02 | 0.08 | 0.11 | 0.000 | 52.93 | 0.01 | 46.38 | 0.31 | 0.16 |
| 12 | 0.02 | 0.09 | 0.14 | 0.000 | 48.79 | 0.01 | 50.46 | 0.31 | 0.18 |
| 16 | 0.03 | 0.09 | 0.19 | 0.002 | 57.63 | 0.01 | 41.67 | 0.22 | 0.15 |
| 20 | 0.02 | 0.09 | 0.14 | 0.001 | 48.60 | 0.01 | 50.69 | 0.26 | 0.20 |
| 24 | 0.01 | 0.08 | 0.13 | 0.000 | 49.31 | 0.01 | 50.03 | 0.27 | 0.16 |
| 28 | 0.02 | 0.10 | 0.15 | 0.000 | 55.79 | 0.01 | 43.50 | 0.29 | 0.15 |
| 32 | 0.01 | 0.09 | 0.10 | 0.000 | 54.70 | 0.01 | 44.52 | 0.40 | 0.18 |
| 36 | 0.01 | 0.09 | 0.08 | 0.000 | 57.21 | 0.00 | 42.02 | 0.39 | 0.20 |
| 40 | 0.01 | 0.10 | 0.16 | 0.000 | 59.09 | 0.01 | 40.24 | 0.22 | 0.16 |
| 44 | 0.01 | 0.09 | 0.13 | 0.000 | 58.94 | 0.01 | 40.31 | 0.31 | 0.20 |
| 48 | 0.01 | 0.08 | 0.09 | 0.000 | 56.07 | 0.01 | 43.24 | 0.33 | 0.17 |
| 52 | 0.01 | 0.09 | 0.13 | 0.000 | 58.94 | 0.01 | 40.31 | 0.31 | 0.20 |
| Avg. | 0.014 | 0.091 | 0.129 | 0.000 | 54.831 | 0.005 | 44.448 | 0.303 | 0.178 |

### Example 6: Iodine Absorption into Toluene

Six studies on iodine (I₂) absorption using toluene as a solvent were performed. Each study was conducted after reaching steady-state reactor conditions during different continuous runs for the decomposition of trifluoroacetyl iodide (TFAI), during which the average conversion of trifluoroacetyl iodide (TFAI) was about 65% and the reaction pressure was 25 psig.

In each case, the trifluoroacetyl iodide (TFAI) reactor effluent stream was directed to a collection system consisting of a 950 ml high pressure Fisher-Porter (F-P) Tube containing toluene (i.e., a toluene bubbler), followed by a second 950 ml high pressure F-P Tube dry ice trap. In each experiment, the 950 ml toluene bubbler was initially charged with 300 grams of toluene and heated to 45-50° C. by wrapping electrical heat tape loosely around the bubbler. The 950 ml dry ice trap (DIT) was placed in a Dewar containing an acetone/dry ice slush at −81° C. The reactor effluent was fed to the 950 ml F-P tube toluene bubbler through a dip tube. The toluene then absorbed the majority of the iodine (I₂), and the majority of the trifluoroacetyl iodide (TFAI) was condensed.

The stream exiting the top of the bubbler was substantially free of iodine (I₂). The iodine concentration may be determined by titration. An example of this method is to add a known amount of sample to 36 grams of deionized water, mixing, adding 4.0 grams of potassium iodide (KI), and titrating the mixture with sodium thiosulfate. This iodine (I₂)-free stream was fed to the second 950 ml F-P tube dry ice trap in which trifluoroiodomethane (CF₃I), unreacted trifluoroacetyl iodide (TFAI), and by-products were collected (including entrained or volatized toluene). The exit stream from the dry ice trap was then directed to the trifluoroiodomethane (CF₃I) crude column for about 16 hours to allow the carbon monoxide (CO) produced in the course of the reaction to vent and avoid pressure build-up in excess of 25 psig in the experimental apparatus.

The initial and final weight of the trifluoroacetyl iodide (TFAI) feed cylinder was recorded and the total amount of material collected in the bubbler and dry ice traps was recorded. The toluene and additional material remaining in the bubbler, as well as the material collected in the dry ice traps, were sampled and the iodine (I₂) concentration of each was determined by titration. Data for each of the experiments is shown below in Table 12 for runs number 1-12, in which the net weight of the bubbler includes the 300 g of toluene with which it was charged at the beginning of the run. As shown in Table 1, the toluene bubbler absorbed between 93.85% and 98.45% of the iodine (I₂) in the reactor effluent stream.

The samples were also analyzed by gas chromatography/mass spectrometry (GC/MS) which verified that very little reaction between the toluene and reactor effluent material had occurred (only single digit ppm levels of by-products attributed to the possible reaction with toluene were found).

TABLE 12

| | | Bubbler | | | Dry ice trap | | | | |
|---|---|---|---|---|---|---|---|---|---|
| | TFAI | | Conc. | I₂ | | Conc. | I₂ | Total | % |
| | fed, | Contents | I₂, | Captured | Contents | I₂, | Captured, | I₂, | I₂ in |
| # | g | Wt., g | ppm | g | Wt., g | ppm | g | g | bubbler |
| 1 | 1907 | 563 | 12960 | 7.3 | 1473 | 78 | 0.11 | 7141 | 98.45 |
| 2 | 1816 | 629 | 14394 | 9.0 | 1318 | 130 | 0.17 | 9.17 | 98.13 |
| 3 | 2043 | 684 | 6143 | 4.2 | 1488 | 67 | 0.10 | 4.30 | 97.68 |
| 4 | 1861 | 681 | 6150 | 4.2 | 1310 | 92 | 0.12 | 4.32 | 97.20 |
| 5 | 1861 | 592 | 11299 | 6.7 | 1389 | 98 | 0.14 | 6.84 | 98.01 |
| 6 | 1861 | 529 | 10510 | 5.6 | 1402 | 260 | 0.36 | 5.96 | 93.85 |

Table 13, below, shows the mass balance and theoretical yield of both trifluoroiodomethane (CF₃I) and carbon monoxide (CO) for each run, calculated using 65% conversion of trifluoroacetyl iodide (TFAI). The mass balance for each run is also shown.

TABLE 13

| Run | Theoretical CF₃I (g) | Theoretical CO (g) | Mass balance (%) |
|---|---|---|---|
| 1 | 1084.5 | 155.0 | 99.2 |
| 2 | 1032.8 | 147.6 | 98.8 |
| 3 | 1161.9 | 166.1 | 99.8 |
| 4 | 1058.4 | 151.3 | 99.0 |
| 5 | 1139.8 | 162.9 | 99.1 |
| 6 | 1112.2 | 159.0 | 98.6 |

### Example 7: TFAI and Toluene Recovery

The toluene was recovered from material collected from a toluene bubbler in an experiment similar to those described in Example 6. The recovery experiment was conducted in a 20-stage Oldershaw glass distillation column equipped with a magnetic splitter. The toluene bubbler material (526 g) was charged to a 1 L glass round bottom flask reboiler. The reboiler was placed in an electric heating mantle and gently heated to drive off non-condensables and ‘lights’. Reflux was observed when the head temperature was between 25° C. and 29° C. The material collected in the product receiver through the vapor line at this temperature was designated the Lights Cut. The material was pink, was estimated to have a volume roughly 30 ml, and was not further quantified.

When the head temperature reached 30° C. and there was sufficient reflux, a distillate cut was taken with a splitter timer of 10 seconds reflux to 1 second take-off and was designated as Main Cut #1. All of the material collected in the distillate receiver at a head temperature between 30° C. and 35° C. A total of 157.3 grams of pink liquid was collected. The gas chromatography (GC) analysis showed that the material consisted of 98.3% trifluoroacetyl iodide (TFAI) with trifluoroiodomethane (CF3I), trifluoroacetic acid (TFA), and toluene also present in minor amounts. The concentration of iodine (I2) was determined by titration was 378 ppm. This result showed that the trifluoroacetyl iodide (TFAI) can be successfully recovered from the toluene while leaving the majority of the dissolved I₂ behind.

Next, an intermediate distillate cut of material was taken when the head temperature was between 35° C. and 110° C. using the same splitter setting. 28.1 g was collected in the distillate receiver and the material was dark purple. The GC analysis showed that the material consisted of 99.6 GC area % toluene including some trifluoroacetyl iodide (TFAI) and trifluoroacetic acid (TFA). The iodine concentration was not determined.

When the head temperature was steady at 110° C. with sufficient reflux, a second main distillate cut was collected and designated as Main Cut #2. The same splitter setting was used. The cut was collected over about 12 hours, until the reboiler temperature reached 117° C. A total of 115.1 grams of orange liquid was collected. The reboiler temperature during this cut was between 112.5° C. and 117° C., and the head temperature ranged from 110° C. to 110.5° C. GC analysis showed the material consisted of greater than 99.9 GC area % toluene. The iodine concentration as determined by titration was 564 ppm. This result showed that toluene can be successfully recovered from the toluene/I₂ mixture while leaving the majority of the dissolved iodine behind.

The reboiler residue was drained from the flask and amounted to 94.5 g. GC analysis was not performed on the material, but it was analyzed for iodine concentration by titration, which showed 11,049 ppm iodine. Table 14 shows the pertinent data for the toluene recovery experiment.

TABLE 14

| | | Head | | | | |
|---|---|---|---|---|---|---|
| Sample | Amount | Temp | GC % | I₂ | I₂ | |
| description | (g) | (° C.) | area | (ppm) | (g) | Notes |
| Starting | 526 | | N/A | >2000 | — | Bubbler material is |
| material | | | | | | dark purple |
| Lights cut | 50 | <30 | N/A | N/A | N/A | Pink material |
| | (est.) | | | | | |
| Main Cut 1 | 157.3 | 30-35 | 98.3 TFAI, | 378 | 0.0595 | Pink material |
| | | | 0.98 CF₃I, | | | |
| | | | 0.15 TFA, | | | |
| | | | 0.48 | | | |
| | | | toluene, | | | |
| | | | 0.09 other | | | |
| Intermediate | 28.1 | 35-110 | 99.63 | N/A | N/A | Purple material; |
| Cut | | | toluene, | | | column bumped |
| | | | 0.16 TFAI, | | | during collection |
| | | | 0.0292 | | | |
| | | | TFA, | | | |
| | | | 0.18 others | | | |
| Main Cut 2 | 115.1 | 110-110.5 | >99.9% | 564 | 0.0649 | Orange material |
| | | | toluene | | | |
| Reboiler | 94.5 | — | Not | 11049 | 1.0441 | Dark purple |
| Residue | | | determined | | | material |

### Example 8: TFAI and Toluene Recovery

A second experiment was conducted to recover the toluene from the material collected from one of the toluene bubblers used in Example 6. The same batch glass distillation apparatus was used as in Example 7. The toluene bubbler material collected in Run 2 of Example 6 (558.1 g) was charged to the toluene recovery apparatus consisting of a 1 L glass round bottom flask reboiler. The iodine concentration was 14,304 ppm. The reboiler was placed in an electric heating mantle and gently heated to drive off non-condensables and ‘lights’. After a short time, reflux was observed at a head temperature of 25° C. to 29° C. A very small amount of material (0.9 grams) was collected in the product receiver through the vapor line and was designated as the “Lights Cut”. An attempt to transfer the lights cut to a cylinder was unsuccessful; therefore, no analysis was performed on this cut.

When the head temperature reached 30° C. with sufficient reflux, a distillate cut was taken with a splitter timer of 10 seconds reflux to 1 second take-off and was designated as Main Cut #1 and was collected in the distillate receiver at a head temperature range of 30° C. to 32° C. A total of 208.4 grams of liquid with a slight pink color was collected. The iodine concentration as determined by titration was 153 ppm. A gas chromatography (GC) analysis showed 97.17 GC area % trifluoroacetyl iodide (TFAI), 1.38 GC area % toluene, 1.12 GC area % trifluoroiodomethane (CF3I), and 0.19 GC area % trifluoroacetic acid (TFA).

Next, an intermediate cut of material was collected with the same splitter setting at a head temperature of 32° C. to 110° C. The coral-colored liquid (57.3 g) was collected in the distillate receiver. The iodine concentration as determined by titration was 451 ppm. A GC analysis showed 99.61GC area % toluene, 0.23 GC area % trifluoroacetyl iodide (TFAI), and 0.11 GC area % trifluoroacetic acid (TFA).

When the head temperature was steady at 110° C. with sufficient reflux, a second main distillate cut was collected and designated as Main Cut #2. The same splitter setting was used. During this cut, the head temperature ranged from 110° C. to 110.5° C., and the reboiler temperature ranged from 112.5° C. to 200° C. A light pink-colored liquid (196.1 g) was collected in the distillate receiver. The iodine concentration as determined by titration was 202 ppm. GC analysis showed 99.996 GC area % toluene. These results showed that toluene can be successfully recovered from the toluene/I₂ mixture while leaving the majority of the dissolved iodine behind.

GC analysis of the dark purple residue in the reboiler was attempted. However, the concentration of iodine was high enough that solid crystals could be visually observed in the reboiler, and further addition of toluene was not sufficient to remove them all. Due to the large amount of iodine present, GC analysis could not be completed.

Table 15 shows the data for this toluene recovery experiment.

TABLE 15

| | | Head | | I₂ | | |
|---|---|---|---|---|---|---|
| Sample | Amount | T | GC | conc. | I₂ | |
| Description | (g) | (° C.) | % area | (ppm) | (g) | Notes |
| Starting | 558.1 | — | N/A | 14,304 | 7.9831 | Dark purple |
| material | | | | | | material |
| Lights Cut | 0.9 | <30 | N/A | N/A | N/A | Insignificant |
| | | | | | | amount |
| | | | | | | collected |
| Main Cut 1 | 208.4 | 30-32 | 97.17 TFAI, 1.38 | 153 | 0.0319 | Light pink |
| | | | toluene, | | | material |
| | | | 1.12CF₃I, 0.19 | | | |
| | | | TFA | | | |
| Intermediate | 57.3 | 32-110 | 99.61 toluene, | 451 | 0.0258 | Coral-colored |
| Cut | | | 0.23 TFAI, 0.11 | | | material |
| | | | TFA | | | |
| Main Cut 2 | 196.1 | 110-110.5 | 99.996 toluene | 202 | 0.0396 | Light pink |
| | | | | | | material |
| Reboiler | 54.03 | — | N/A | 57,490 | 3.1062 | Deep purple |
| residue | | | | | | material |
| Mass balance | 92.6 | | | | | |

### Comparative Example 9: TFAI Purification

This example illustrates a larger laboratory-scale batch distillation of crude TFAI. The distillation column consisted of a 10 gallon reboiler, a two-inch inner diameter, ten foot-long column with Propak column packing, and a shell and tube condenser. The column had about 30 theoretical plates. The distillation column was equipped with a reboiler level indicator, and temperature, pressure, and differential pressure transmitters.

Multiple relatively large-scale laboratory TFAI crude batch distillations were performed. The crude TFAI included TFAI, I₂, HI₃, trifluoroacetic acid, trifluoroacetyl chloride, and minor amounts of low- and high-boiling impurities, relative to TFAI.

In an example of a typical batch distillation, the column reboiler was charged with 110 lbs of crude TFAI consisting of TFAI, I₂, HI₃, trifluoroacetic acid, trifluoroacetyl chloride, and minor amounts of low- and high-boiling impurities, relative to TFAI. After some non-condensables and lights were vented to a caustic scrubber, reflux was established, and the main cut distillate take-off commenced. The distillation operating conditions are shown in Table 16.

TABLE 16

| Process | Conditions | |
|---|---|---|
| Pressure | 10.0-25.0 | psia |
| Change in pressure | 25-28″ H₂O | |
| O/H draw-off rate (lights venting) | 0.1-0.5 lb/hr (to house scrubber) | |
| O/H draw-off rate (main cut) | 0.5-0.7 | lb/hr |

| | |
|---|---|
| Column O/H temp. (lights venting) | 0-10° C. (pressure dependent) |
| Column O/H temp. (main cut) | 28-38° C. (pressure dependent) |
| Reboiler temp. (lights venting) | 0-10° C. (pressure dependent) |
| Reboiler temp. (main cut) | 28-65° C.* (pressure dependent) |

*Distillation is ended when reboiler temperature reaches 35-65° C.

The distillation was ended after 101 lbs was collected in the product collection cylinder and the reboiler temperature reached 40° C. The purified TFAI was sampled and analyzed for I₂ and HI₃ concentration, and by gas chromatography/mass spectrometry—flame ionization detection (GC/MS-FID) for TFAI purity. A typical I₂ concentration over multiple distillations was 1310 ppm, a typical HI₃ concentration was 650 ppm, and a typical TFAI purity was a 99.15 FID area % as shown in Table 17 below.

TABLE 17

| | Amount | Head T | GC | I₂ conc. | I₂ | |
|---|---|---|---|---|---|---|
| Sample | (g) | (° C.) | % area | (ppm) | (g) | Notes |
| Starting | 558.1 | — | N/A | 14,304 | 7.9831 | Dark purple |
| material | | | | | | material |
| Lights Cut | 0.9 | <30 | N/A | N/A | N/A | Insignificant |
| | | | | | | amount |
| | | | | | | collected |
| Main Cut 1 | 208.4 | 30-32 | 97.17 TFAI, | 153 | 0.0319 | Light pink |
| | | | 1.38 toluene, | | | material |
| | | | 1.12CF₃I, | | | |
| | | | 0.19 TFA | | | |
| Inter- | 57.3 | 32-110 | 99.61 toluene, | 451 | 0.0258 | Coral- |
| med.Cut | | | 0.23 TFAI, | | | colored |
| | | | 0.11 TFA | | | material |
| Main Cut 2 | 196.1 | 110-110.5 | 99.996 | 202 | 0.0396 | Light pink |
| | | | toluene | | | material |
| Reboiler | 54.03 | — | N/A | 57,490 | 3.1062 | Deep purple |
| residue | | | | | | material |
| Mass | 92.6 | | | | | |
| balance | | | | | | |

### Example 10: TFAI Purification from Solvent

The same batch distillation column that was used in Comparative Example 9 was used to perform a purification of crude TFAI. The crude TFAI consisted of TFAI, I₂, HI₃, trifluoroacetic acid, trifluoroacetyl chloride, and minor amounts of low boiling and hi-boiling impurities, relative to TFAI and was the produced by the same method and was of the same quality of that used in same Comparative Example 9.

The batch distillation column reboiler was charged with 127 lbs of TFAI crude. After some non-condensibles and lites were vented to a caustic scrubber the distillation was shut down for two days, and 3 gallons of reagent grade toluene were charged to the reboiler. The distillation was restarted and the main cut proceeded normally. The operating conditions were the same as those in Comparative Example 9, with the only difference being that the reboiler temperature was warmer than the column temperatures at the start, and slowly increased as more TFAI was collected overhead. The distillation was ended after 113 lbs was collected in the product collection cylinder (111 lbs) and samples (2 lbs). The reboiler temperature at the time the distillation was ended was 94° C. and the column was under a slight vacuum. There was still some TFAI that could have been recovered based on the 110° C. boiling point of toluene. The overhead stream was spot-checked and sampled twice during the course of the distillation. The first sample (sample #1) was taken after about 35 lbs was collected and sample #2 was taken after 80 lbs. Both samples were semi-translucent purple, with sample #2 the clearest, and both having much less color than the typical main cut without added toluene as can be seen in the figure below.

The spot-checked TFAI samples TFAI were analyzed for I₂ and HI₃ concentration, and by GCMS-FID for TFAI purity. The I₂ concentrations of the two samples were 513 ppm and 644 ppm respectively, representing a 61% and 51% reduction in I₂ concentration over a typical purified TFAI material without the presence of toluene. The HI₃ concentrations of the two samples were 254 ppm and 493 ppm respectively, representing a 61% and 24% reduction in HI₃ concentration over a typical purified TFAI material without the presence of toluene. The TFAI purity of both samples (99.88 and 99.72 GC FID area %) is superior to typical purified TFAI material without the presence of toluene (99.15 GCMS FID area %). The results are shown below in Table 18.

TABLE 18

| | I₂ Titration | | TFAI Purity |
|---|---|---|---|
| | I₂ Conc. | HI₃ conc. | GCMS-FID |
| Sample #/Description | ppm | ppm | Area % |
| Typical I₂ and HI₃ conc. w/out | 1310 | 650 | 99.15 |
| added toluene | | | |
| Toluene distillation #1, O/H | 513 | 254 | 99.88 |
| Sample 1 with added toluene | | | |
| Toluene distillation #1, O/H | 644 | 493 | 99.72 |
| Sample 2 with added toluene | | | |
| Average, Samples 1 and 2 | 578.5 | 373.5 | 99.8 |

### Example 11: Toluene and Iodine (I₂) Reactivity at 80° C. and 250° C.

A mixture of 10 wt. % iodine and 90 wt. % toluene was charged to a 600 ml autoclave, mixed, and heated to 80° C. for 24 hours. The liquid was then sampled via a dip tube, and analyzed by 1H NMR and gas chromatography/mass spectrometry (GC/MS) to determine whether any iodine-containing compounds were present.

In a second run, a mixture of 10 wt. % iodine and 90 wt. % toluene was again charged to a 600 ml autoclave, mixed, and heated to 250° C. for 2 weeks, after which no increase in pressure was observed. The liquid was then sampled via a dip tube and analyzed by ¹H NMR and GC/MS to see whether any iodine-containing compounds were present.

The experimental data can be found in Table 19 below.

TABLE 19
Iodine and toluene thermal stability

| I₂ | Toluene | | |
|---|---|---|---|
| (wt. %) | (wt. %) | Temp. and duration | Result |
| 10 | 90 | 80° C., 24 hr | No Reaction |
| 10 | 90 | 250° C., 2 weeks | No Reaction |

Based on ¹H NMR and GC/MS, it was concluded that no reaction occurs between toluene and iodine (I₂) at 80° C. after 24 hours, nor at 250° C. after two weeks. Although no iodinated toluene species or other iodine-containing species were detected by GC/MS after two weeks at 250° C., small amounts (<0.5%) of benzene, xylene, p-xylene were detected, although the exact mechanisms by which these species are formed are not known at this time.

### Example 12: Drying and Purification of Crude CF₃I

One thousand pounds of wet and acid-free crude CF₃I vapor from the caustic scrubber outlet is condensed in a condenser. The condensed wet CF₃I will then flow into a decanter. The water will settle as top layer while the CF₃I will settle as bottom layer.

The top water layer is withdrawn and is expected to include about 1.89 lbs of water and about 1200 ppm of dissolved CF₃I or 0.002 lbs. This water can be recycled to the caustic scrubber for organic recovery or be disposed of.

The bottom organic layer including CF₃I is withdrawn and is expected to have about 1,000 lbs of CF₃I and to contain about 130 ppm of dissolved water or 0.13 lbs. This resulting CF₃I stream is then dried with a drying agent such as 3 Angstrom or 4 Angstrom molecular sieves, activated alumina, silica gel, calcium sulfate (CaSO₄), and the like.

Using a commercial 3 Angstrom molecular sieve desiccant which can adsorb up to 15% moisture, this improved process would have consumed only 0.87 pounds of molecular sieve for every 1,000 pounds of CF₃I processed. The water content is about 10 ppm after this treatment. In view of this low desiccant consumption rate, the drying equipment size can be made much smaller than those used in extant processing methods. Furthermore, given that the molecular sieves can be regenerated, the ultimate drying agent consumption can be minimized.

### Example 13—PTx Study Using Trifluoroacetyl Chloride (TFAC) and Sulfur Dioxide (SO₂): 10° C. Isotherm

A set of volume calibrated PTx cells were used to measure azeotrope and azeotrope-like compositions of trifluoroacetyl chloride (TFAC) and sulfur dioxide (SO₂). Mixtures of TFAC and SO₂ were gravimetrically prepared into evacuated PTx cells; two cells were reserved for measuring each pure component. Once prepared, each of up to eight cells of differing compositions were inserted into a thermostatted chamber. In the chamber, each cell was attached to an instrumentation manifold equipped with calibrated pressure transducers and resistance temperature detectors (RTD); this provided a means to measure and record the total saturation pressure of each cell's contents at its local temperature.

To establish equilibrium at a target temperature, the set point of the chamber was adjusted to an average temperature (T_{avg}) of 10° C. Once at equilibrium, recognized as when temperature and pressures of each cell remain stable for several hours, the local temperature and saturation pressures of each cell were recorded. From these pressure-temperature-composition data, the binary interaction parameters of TFAC and SO₂ for the Helmholtz Energy Equation of State (HEOS) were identified. A minimum boiling azeotrope was formed with a composition of about 75.0 wt. % TFAC and about 25.0 wt. % SO₂ was formed, and data is presented below in Table 20.

TABLE 20

| TFAC Composition (mass %) | Pressure (psia) |
|---|---|
| 0.0% | 33.4 |
| 24.5% | 44.6 |
| 47.9% | 50.6 |
| 73.8% | 53.2 |
| 84.7% | 52.0 |
| 89.7% | 51.0 |
| 94.9% | 48.9 |
| 100.0% | 44.7 |

### Example 14—SO₂ Adsorption Efficiency of Various Solid Adsorbents

The adsorption column was charged with about 50 mL of pre-weighed selected solid adsorbent. A 500 mL stainless steel cylinder was charged with about 300 g of 0.1069 wt. % SO₂-containing trifluoroacetyl chloride (TFAC). After the system was pressure checked, SO₂-containing TFAC was then circulated through the adsorption column by a recirculation pump at room temperature (20° C. to 30° C.). After 24 hours, the recirculation pump was stopped, and a TFAC sample was taken for analysis to determine the concentration of SO₂. The SO₂ removal efficiency was then calculated.

The tested solid adsorbents are listed below in Table 21.

TABLE 21

| Run | | Volume of | Weight of |
|---|---|---|---|
| # | Adsorbent | adsorbent, mL | adsorbent, g |
| 1 | UOP XH-9 Mol-Sieve | 50 | 42.28 |
| 2 | BASF SAS40 1/8″ Alumina | 50 | 36.60 |
| 3 | Zeolyst CBV5524G CY(1.6) | 50 | 30.10 |
| 4 | NORIT ROX0.8 Activated Carbon | 50 | 16.58 |
| 5 | Silica Gel | 50 | 36.13 |
| 6 | Grace Davison 10A Mol-Sieve | 50 | 32.42 |
| 7 | Mol-Sieve 4A (UOP) | 46 | 30.33 |
| 8 | Acros organics 3A Mol-Sieve | 50 | 35.30 |
| 9 | Osaka Gas Chemicals MSC-3K | 50 | 32.44 |
| | 172 carbon Mol-Sieve | | |

Table 22 below, shows the removal efficiency of the different adsorbents. All tested solid adsorbents showed some degree of SO₂ removal capacity, with SO₂ completely adsorbed by Osaka Gas Chemicals MSC-3K 172 carbon Mol-Sieves.

TABLE 22

| | | SO² concentration | | | g SO₂ |
|---|---|---|---|---|---|
| | | in TFAC, wt. % | | | adsorbed/ |
| | TFAC | Before | After | SO₂ | 100 g |
| Run # | used, g | adsorption | adsorption | removal, % | adsorbent |
| 1 | 367.14 | 0.1069 | 0.0983 | 8.04 | 0.07 |
| 2 | 329.23 | 0.1069 | 0.0934 | 12.63 | 0.12 |
| 3 | 310.18 | 0.1069 | 0.0922 | 13.75 | 0.15 |
| 4 | 335.46 | 0.1069 | 0.0752 | 29.65 | 0.64 |
| 5 | 319.95 | 0.1069 | 0.0625 | 41.53 | 0.39 |
| 6 | 329.36 | 0.1069 | 0.0509 | 52.39 | 0.57 |
| 7 | 311.35 | 0.1069 | 0.0344 | 67.82 | 0.74 |
| 8 | 332.26 | 0.1069 | 0.0337 | 68.48 | 0.69 |
| 9 | 293.39 | 0.1069 | 0.0000 | 100.00 | 0.97 |

### Example 15—Separation and Purification of Trifluoroacetyl Chloride (TFAC)

A composition including crude trifluoroacetyl chloride (TFAC), sulfur dioxide (SO₂), and at least one additional impurity is provided. In a first step, the relative amounts of trifluoroacetyl chloride (TFAC) and sulfur dioxide (SO₂) are adjusted by adding trifluoroiodomethane (TFAC) to the composition, adding sulfur dioxide (SO₂) to the composition, or adding both trifluoroacetyl chloride (TFAC) and sulfur dioxide (SO₂) to the composition. The composition is then exposed to effective conditions such that an azeotrope or azeotrope-like mixture is formed. The azeotrope or azeotrope-like mixture may then be separated from the at least one impurity by distillation, phase separation, or fractionation. Once the azeotrope or azeotrope-like mixture is separated from the impurity, the components of the azeotrope or azeotrope-like mixture—trifluoroacetyl chloride (TFAC) and sulfur dioxide (SO₂)—are separated from one another in a second step. The separation of trifluoroacetyl chloride (TFAC) and sulfur dioxide (SO₂) may then be accomplished by distillation, exposure to a solid adsorbent, or a combination thereof.

### Example 16—Distillation of Trifluoroacetyl Chloride (TFAC)

A composition including crude trifluoroacetyl chloride (TFAC) and sulfur dioxide (SO₂) is provided. The composition is conveyed to a distillation column. The distillate, which may comprise sulfur dioxide (SO₂), trifluoroacetyl chloride (TFAC), or a mixture thereof, is collected. The bottoms product, which comprises trifluoroacetyl chloride (TFAC) may be collected. The amount of sulfur dioxide (SO₂) present in the bottoms product may be 100 ppm or less, 50 ppm or less, 10 ppm or less, or 1 ppm or less.

### Example 17—Sulfur Dioxide (SO₂) Removal from Trifluoroacetyl Chloride (TFAC) by Batch Distillation

52.73 lbs of 740 ppm SO₂-containing TFAC was charged into a distillation unit equipped with a 10-gallon reboiler, a 2″ ID×120″ L column (packed with Goodloe 2-inch diameter by 6-inch thick structured metal packing) and a tube in shell condenser (10.45 ft² of surface area) for a batch distillation to remove SO₂ from TFAC. The reboiler was heated up to about 40° C. with 30 psig steam/city water mixture. During the operation, every 2-4 hours, 2-4 psig of column pressure was vented-off to a lights collection cylinder from the column overhead to remove non-condensable gases at the startup and to purge out concentrated SO₂ from the system. The overhead reflux and reboiler samples were taken periodically for SO₂ analysis using a pre-calibrated thermal conductivity detection gas chromatograph (TCD-GC). Based on the results, SO₂ contained in the TFAC was concentrated into the column overhead. With the overhead purge continued, the reboiler SO₂ concentration continued to drop and eventually reached below GC detection limit (less than 5 ppm). After eight reboiler samples showed an amount of SO₂ below the GC detection limit (<5 ppm), the reboiler material was fully drained to a heavies collection cylinder with 49.67 lbs of purified TFAC collected containing <5 ppm SO₂ (below GC detection limit) representing a yield of 96.84%. The lights collection cylinder gained 1.53 lbs during the operation which gave a total mass balance of 99.82%.

A second batch distillation was conducted in the same distillation unit as described above. 54.70 lbs of 958 ppm SO₂-containing TFAC was charged into the reboiler. During the operation, every 2-4 hours, 2-4 psig of column pressure was vented-off to a lights collection cylinder from the column overhead to remove non-condensable gases at the startup and to purge out concentrated SO₂ from the system. The overhead reflux and reboiler samples were taken periodically for SO₂ analysis using a pre-calibrated TCD-GC. Based on the results, SO₂ contained in the TFAC was concentrated into the column overhead stream. With the overhead purge continued, the reboiler SO₂ concentration continued to drop and eventually reached below GC detection limit (less than 5 ppm). After three reboiler samples showed an amount of SO₂ below the GC detection limit, the reboiler material was fully drained to a heavies collection cylinder with 53.93 lbs of purified TFAC collected containing <5 ppm SO₂ (below GC detection limit) representing a yield of 98.59%. The lights collection cylinder gained 0.49 lbs during the operation which gave a total mass balance of 99.49%.

### Example 18: Purification of Trifluoroacetyl Chloride (TFAC) by Distillation

A composition including trifluoroacetyl chloride (TFAC) and 2500 ppm sulfur dioxide (SO₂) is purified to provide a purified stream of TFAC containing 5 ppm SO₂.

1000 lbs/hr of crude TFAC containing 997.5 lb/hr of TFAC and 2.5 lb/hr SO₂ is fed to a distillation column operating at 52.7 psia at the top of the column. The distillation column has 40 stages, and is fitted with a condenser cooled with chilled water with a supply temperature of 5° C. such that the overhead temperature of the column operates at about 10° C. The reboiler is heated with steam (e.g. 10 psig saturated steam at 115° C.). At a reflux rate of 1540 lb/hr and boilup rate of 1820 lb/hr, a bottoms stream of purified TFAC containing 5 ppm SO₂ is recovered. The overhead and distillate streams are concentrated to the approximate azeotropic composition of TFAC and SO₂ (about 76 wt. % TFAC, total distillate flowrate 10.25 lb/hr). The distillation yield of TFAC is over 99.2%. The feed rates and weight percentages of the components are shown below in Table 23.

TABLE 23

| Component | Feed (wt. %) | Distillate (wt. %) | Bottoms (wt. %) |
|---|---|---|---|
| TFAC | 99.75 | 75.66 | 99.999 |
| SO2 | 2.5 | 24.34 | 0.0005 |

Column conditions are shown below in Table 24.

TABLE 24

| Number of stages | 40 | |
|---|---|---|
| Feed Stage | 20 | |
| Overhead Pressure | 52.7 | psia |
| Overhead Temperature | 10° | C. |
| Bottoms Temperature | 14.8° | C. |

| | |
|---|---|
| Reflux Ratio | 150 |
| Boil up Ratio | 1.84 |

Other conditions, including different numbers of stages, different feed stages, different pressures, different reflux ratios, and different boil up ratios may also be used to purify mixtures of TFAC and SO₂.

### Example 19—Alternative Method for Distillation of Trifluoroacetyl Chloride (TFAC)

A composition including crude trifluoroacetyl chloride (TFAC) and sulfur dioxide (SO₂) is provided. In a first step, the composition is conveyed to a distillation column and exposed to effective conditions such that an azeotrope or azeotrope-like mixture is formed. The bottoms product, which comprises trifluoroacetyl chloride (TFAC) may be collected. The amount of sulfur dioxide (SO₂) present in the bottoms product may be 100 ppm or less, 50 ppm or less, 10 ppm or less, or 1 ppm or less.

The azeotrope or azeotrope-like mixture is collected as the distillate. The components of the azeotrope or azeotrope-like mixture in the distillate—trifluoroacetyl chloride (TFAC) and sulfur dioxide (SO₂)—are separated from one another in a second step. The separation of trifluoroacetyl chloride (TFAC) and sulfur dioxide (SO₂) may then be accomplished by distillation, exposure to a solid adsorbent, or a combination thereof.

### Example 20: Separation of Trifluoroacetyl Chloride (TFAC) from Trifluoroacetyl Iodide (TFAI)

In this Example, the separation of trifluoroacetyl iodide is described. A mixture containing about 80 wt. % trifluoroacetyl iodide, about 10 wt. % trifluoroacetyl chloride, about 5 wt. % hydrogen iodide, and about 5 wt. % hydrogen chloride can be charged into a distillation column. The distillation column can include a 10 gallon reboiler, a 2-inch inside diameter 10-foot Pro-Pak® column from the Cannon Instrument Company, State College, PA, and about 30 theoretical stages. The distillation column can be equipped with temperature, absolute pressure, and differential pressure transmitters. The distillation can be run at a pressure of about 300 kPaG and at a temperature of about 55° C., with hydrogen chloride taken off from the top of the column, and product from the bottom of the column.

### Example 21: Analysis of Treated Trifluoroacetyl Iodide (TFAI)

A 1-inch outer diameter by 9-inch length stainless steel column was charged with 29.2 g fresh Norit ROX 0.8 activated carbon having an iodine number of 1,100 and a BET surface area of 1,225 m²/g. Two 300 mL collection cylinders were prepared. The first cylinder was connected to the exit of the trifluoroacetyl iodide (TFAI) feed line and placed in a Dewar filled with wet ice and set on a balance. The flow of TFAI was started at 0.25 lb/hr and was passed through the activated carbon (AC) column at room temperature. The flow was then directed to a scrubber carboy until liquid was observed entering the carboy, indicating that the entire feed line was liquid-filled. Next, the feed flow path was switched over to the collection cylinder for about 3 hours for a total of about 0.75 pounds as confirmed by the weight increase on the balance. The cylinder was isolated and replaced by the second cylinder, and the flow of TFAI was restarted to the new collection cylinder. The two collection cylinders of TFAI, together with a cylinder of pristine TFAI feed, were subjected to various analyses including I₂ titration and ¹H NMR.

Iodine concentration was determined by adding a sample to 36 grams DI water, mixing, adding 4.0 grams KI, mixing and titrating with sodium thiosulfate. The concentration of hydrogen- and iodine-containing species was determined by Proton NMR (¹H-NMR) method by transferring a sample to a heavy wall, valved, NMR tube containing deuterated chloroform (CDCl₃) with calibrated tetramethylsilane (TMS) standard. The concentrations of identified components in the sample were calculated based on the integration values of their peaks. A 300 MHz field strength was used for the analysis of the samples.

The concentrations of I₂ as well as other hydrogen- and iodine-containing species such as HI and HI₃ were compared before and after treatment with the activated carbon (AC) column. The results of these analyses are shown below in Table 25.

TABLE 25

| | |
|---|---|
| Before | After AC column (ppm) |

| | AC column | Collection | Collection |
|---|---|---|---|
| Species | (ppm) | cylinder 1 | cylinder 2 |
| I₂ | 2883 | 2029 | 1751 |
| HI | 1903 | 0 | 241 |
| HI₃ | 632 | 231 | 209 |

### Example 22—Effects of Treated Feed Stock on Product Selectivity

The effects of using activated carbon (AC) to remove iodine-containing impurities, such as HI and HI₃, as well as iodine (I₂) from the trifluoroacetyl iodide (TFAI) was tested. The decomposition of TFAI to CF₃I was conducted at 300° C., 25 psig, 0.25 lb/h TFAI feed rate, and 4.9 sec contact time. A 1-inch outer diameter by 9-inch length stainless steel column was charged with 29.5 grams of fresh Norit ROX 0.8 activated carbon (AC) was installed in the trifluoroacetyl iodide (TFAI) feed line, along with a by-pass loop around the column. The liquid trifluoroacetyl iodide (TFAI) feed was passed through the column at room temperature (“on stream”). The reactor and the reactor effluent gas chromatography (GC) data was collected over 48 hours. The average selectivity for trifluoroiodomethane (CF₃I) was 99.62%. The main by-product was trifluoromethane (CF₃H).

Next, the same trifluoroacetyl iodide (TFAI) feedstock was left untreated (i.e., bypassing the AC column, “bypass”), and was used as feed for about 48 hours under the same reaction conditions described above. The average selectivity for trifluoroiodomethane (CF₃I) decreased to 99.33%, while selectivity for the same major impurity (trifluoromethane, CF₃H) increased.

The experiment was repeated with a different trifluoroacetyl iodide (TFAI) feedstock and the same trend was observed. Results of the tests can be found below in Table 26. The tests show that the use of AC column installed on the trifluoroacetyl iodide (TFAI) feed line resulted in improved selectivity for trifluoroiodomethane (CF₃I), while trifluoroacetyl iodide (TFAI) conversion remained at comparable levels.

TABLE 26

| | | TFAI | Product selectivity, mol % | | |
|---|---|---|---|---|---|
| Exp. # | AC column | conv., % | CF₃I | CF₃H | Others* |
| 1 | On stream | 66.043 | 99.619 | 0.261 | 0.120 |
| | Bypassed | 65.164 | 99.330 | 0.435 | 0.235 |
| 2 | On stream | 69.154 | 99.346 | 0.402 | 0.252 |
| | Bypassed | 69.608 | 98.924 | 0.678 | 0.398 |

*Others include trifluoroacetyl fluoride, C₂F₅I, etc.

The activated carbon column was removed from the trifluoroacetyl iodide (TFAI) line, and the AC was discharged and weighed. After being used, its weight was 2.7 times its original weight, indicating it had adsorbed significant amounts of species present in the trifluoroacetyl iodide (TFAI) feed. The AC was further analyzed by means of TGA-MS (Thermogravimetric Analysis-Mass Spectrometry) to determine the nature of adsorbed species. As shown in Table 27, the species desorbed during TGA include I₂, HI, and trifluoroacetyl iodide (TFAI). The absence of HI₃ among the detected species could be due to its instability upon heating, during which it may decompose to HI and iodine (I₂).

TABLE 27

| Analyte | m/z | Peak Intensity* | Notes |
|---|---|---|---|
| CF₃ | 69 | 8.36E−11 | CF₃ fragment |
| I | 127 | 7.78E−10 | I fragment |
| HI | 128 | 1.98E−11 | HI molecule |
| CF₃I | 196 | 1.80E−11 | CF₃I molecule or TFAI fragment |
| I₂ | 254 | 8.12E−10 | I₂ molecule |

*The larger the peak intensity, the higher the concentration of the analyte.
**This is more likely representative of a CF₃I fragment from the TFAI molecule, given that the TFAI feed was passed through the AC column.

These results, together with the results from Example 21, indicate that AC can be used to remove I₂, HI, and HI₃ from trifluoroacetyl iodide (TFAI) feedstock.

### Example 23—Separation and Purification of Trifluoroiodomethane (CF₃I)

A composition including crude trifluoroiodomethane (CF₃I), at least one impurity, and water, is purified. In a first step, the relative amounts of trifluoroiodomethane (CF₃I) and water are adjusted. The relative amounts of trifluoroiodomethane (CF₃I) and water may be adjusted by adding water, adding trifluoroiodomethane (CF₃I), or both. The composition is then exposed to effective conditions such that an azeotrope or azeotrope-like mixture is formed. The azeotrope or azeotrope-like mixture may then be separated from the at least one impurity by distillation, phase separation, or fractionation. Once the azeotrope or azeotrope-like mixture is separated from the impurity, the components of the azeotrope or azeotrope-like mixture—trifluoroiodomethane (CF₃I) and water—are separated from one another to purify the trifluoroiodomethane. The separation of trifluoroiodomethane (CF₃I) and water may then be accomplished by distillation, liquid-liquid extraction, or exposure to a drying agent.

### Example 24—Separation of Impurities from Trifluoroiodomethane (CF₃I)

In this Example, a crude composition of trifluoroiodomethane (CF₃I) is provided, including trifluoroacetyl chloride (CF₃COCl) as an impurity, along with one or more other impurities such as trifluoromethane (HFC-23 or R23). The relative amounts of trifluoroiodomethane (CF₃I) and trifluoroacetyl chloride (CF₃COCl) are altered if necessary to form sufficient relative amounts and the composition is subjected to distillation at conditions effective to form and separate an azeotrope or azeotrope-like composition of trifluoroiodomethane (CF₃I) and trifluoroacetyl chloride (CF₃COCl) from the remainder of the composition. The separated azeotrope or azeotrope-like composition of trifluoroiodomethane (CF₃I) and trifluoroacetyl chloride (CF₃COCl) is removed from the remaining crude composition of trifluoroiodomethane (CF₃I) as a light component. The remaining crude composition of trifluoroiodomethane (CF₃I) is then subjected to different temperature and pressure conditions wherein the other impurities such as trifluoromethane (HFC-23) may be separated by further distillation to obtain purified trifluoroiodomethane (CF₃I).

### Example 25—Separation of Impurities from Trifluoroiodomethane (CF₃I)

In this example, a composition is provided which includes trifluoroiodomethane (CF₃I) and at least one impurity such as trifluoromethane (HFC-23), for example. To this composition, trifluoroacetyl chloride (CF₃COCl) is added in a sufficient amount and the composition is subjected to conditions effective to form a composition which is an azeotrope or azeotrope-like composition consisting essentially of, or consisting of, effective amounts of trifluoroacetyl chloride (CF₃COCl) and trifluoroiodomethane (CF₃I), followed by separating the azeotrope or azeotrope-like composition from the impurity by a separation technique such as phase separation, distillation, or fractionation, for example. Thereafter, the azeotrope or azeotrope-like composition of trifluoroiodomethane (CF₃I) and trifluoroacetyl chloride (CF₃COCl) may be subjected to further separation or purification steps to obtain purified trifluoroiodomethane (CF₃I).

### Example 26—Separation of Impurities from Trifluoroiodomethane (CF₃I)

In this example, a composition is provided which includes trifluoroacetyl chloride (CF₃COCl) and at least one impurity such as trifluoromethane (HFC-23), for example. To this composition, trifluoroiodomethane (CF₃I) is added in a sufficient amount and the composition is subjected to conditions effective to form a composition which is an azeotrope or azeotrope-like composition consisting essentially of, or consisting of, effective amounts of trifluoroacetyl chloride (CF₃COCl) and trifluoroiodomethane (CF₃I), followed by separating the azeotrope or azeotrope-like composition from the impurity by a separation technique such as phase separation, distillation, or fractionation, for example. Thereafter, the azeotrope or azeotrope-like composition of trifluoroiodomethane (CF₃I) and trifluoroacetyl chloride (CF₃COCl) may be subjected to further separation or purification steps to obtain purified trifluoroiodomethane (CF₃I).

### Example 27—Stability of Trifluoroiodomethane (CF₃I) in Concentrated Sulfuric Acid (H₂SO₄)

In this example the stability of CF3I in concentrated sulfuric acid (H₂SO₄) is demonstrated.

Into a PFA reactor vessel, 15 ml (25.5818 g) 98 wt % H₂SO₄ was charged. The reaction vessel was heated to 40° C. in an oil bath. Temperature was maintained at 40° C. for 30 min before starting the addition of CF3I in order to ensure that the H₂SO₄ was uniformly heated to 40° C. Magnetic stirring was applied to the reactor vessel throughout the experiment to ensure constant temperature, and provide mixing of added CF₃I to H₂SO₄.

To ensure liquid delivery of CF₃I to reactor vessel, the CF₃I cylinder was inverted and ¼″ transfer lines attached with outlet below the surface of H₂SO₄. The outlet of reactor vessel was connected to a PFA trap containing 20 ml (20.0250 g) DI water (deionized water) to remove any entrained H₂SO₄ or any acidic materials that could be formed reaction of CF₃I with H₂SO₄.

Samples of H₂SO₄ reaction mixture were removed at 1, 2, and 4 hr intervals and 19F NMR spectra were acquired in triplicate (3×) for each sampling interval.

Samples of the gas effluent from the reactor vessel were also sampled into Tedlar gas bags at 1, 2, and 4 hr intervals and submitted for GCMS analysis. At the end of experiment, DI water trap contents wer transferred to a glass vial and submitted for IC analysis.

19F NMR spectra showed no additional peaks other than CF₃I and the internal standard, indicating that CF₃I is stable in H₂SO₄ under the testing conditions.

GCMS analysis showed that the purity of the initial CF3I sample and samples obtained exposure time to H₂SO₄ did not show any appreciable change, indicating essentially no reaction between CF3I and H₂SO₄ under the testing conditions. Analyses showed no new impurities. The results of the analyses are shown in Table 28 below.

TABLE 28

GCMS Area % FID

| Component Name | initial | 1 HR | 2 HR | 4 HR |
|---|---|---|---|---|
| CF3I | 99.9886 | 99.9896 | 99.9898 | 99.9913 |
| iodomethane | 0.0022 | 0.0026 | 0.0018 | 0.0012 |
| Others | 0.0092 | 0.0078 | 0.0084 | 0.0075 |

IC analysis showed negligible breakdown of the CF₃I based on the amounts of iodide and fluoride present in the DI water sample after the test. Surprisingly, CF3I was found to be relatively stable in concentrated sulfuric acid.

### Example 27b: Purifying Trifluoroiodomethane (CF₃I) by Scrubbing and Drying the Product Stream with Sulfuric Acid

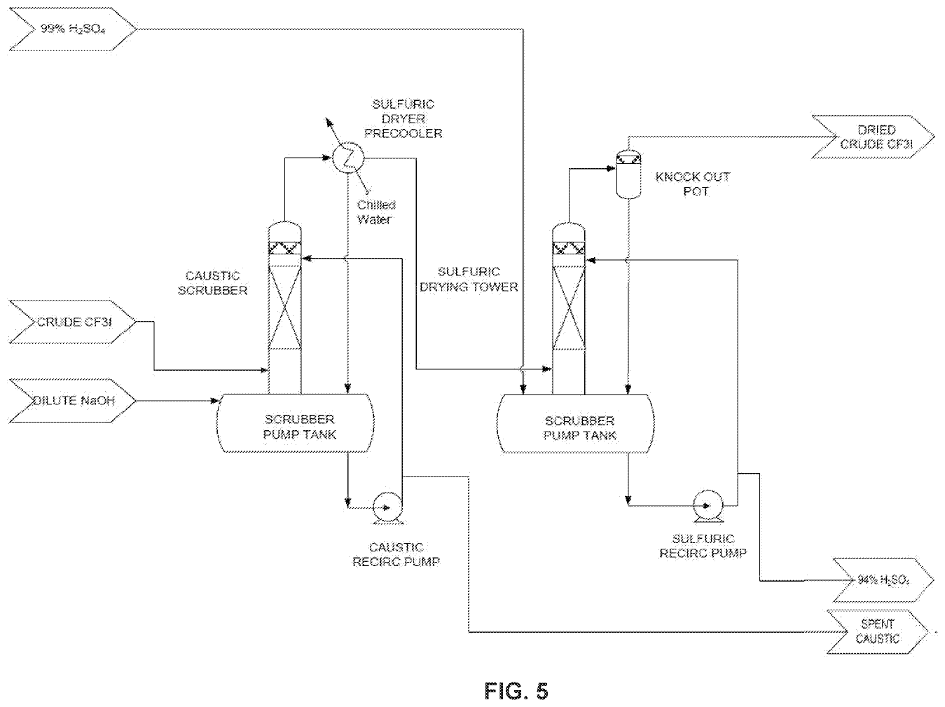

A crude feed stream comprising 1000 lb/hr CF₃I vapor including residual acid may be fed to a circulating caustic scrubber operating at 10 psig. The resulting wet and acid-free crude CF3I vapor may be cooled to 15° C. to condense out water resulting in vapor stream with reduced water content. This stream comprising CF3I and water may be contacted with a concentrated sulfuric acid solution. Water may be preferentially absorbed into the sulfuric acid, resulting in a product stream of CF₃I that is substantially free of water. The feed stream comprising CF₃I and water may be contacted by the sulfuric acid in a contacting tower in which the feed stream comprising CF₃I and water may be present as vapor flowing in a countercurrent manner to the liquid sulfuric acid. When the concentration of sulfuric acid decreases to 94% by weight, it is discharged and a new charge of 99 wt % sulfuric acid is added. The process flow diagram for the process is shown in FIG. 5.

Table 29 below shows typical (exemplary) conditions for drying CF3I using concentrated sulfuric acid (H2SO4).

TABLE 29

| | Inlet | Outlet |
|---|---|---|
| Temperature, ° C. | 15 | 15.6 |
| Pressure, psig | 10 | 9.5 |
| Crude CF3I flow, lb/hr | 1000 | 998.8 |
| Water flow, lb/hr | 0.93 | 0.025 |
| (based on saturation at | | |
| temperature & pressure) | | |

It is to be understood by those skilled in the art that other operating conditions such as higher or lower temperature and/or higher or lower pressure may be employed. Although 98-99 wt % sulfuric acid is used in this example, other concentrations below or above may be used.

### Example 28—Conversion of TFAI to CF₃I at High Pressure

In this Example, a high operating pressure TFAI decomposition reaction is used to produce CF₃I and CO.

A set of two experiment pairs to directly compare low pressure (LP) and high pressure (HP) reactor performance were run using a purified TFAI crude feedstock. For each LP/HP pair, the continuous laboratory vapor phase reactor was first started at a LP of 25 psig and 410° C. and brought to steady operating conditions and run under those conditions for about two days. During this time, reactor exit samples were taken at regular four-hour intervals and analyzed by gas chromatography (GC). Then the reactor pressure was increased to 220 psig over one hour without stopping the feed and the reactor temperature was adjusted to 350° C. to achieve the desired TFAI conversion; i.e., a conversion comparable to what was achieved at the LP conditions, or about 65-70%.

For the first pair of LP/HP experiments the LP portion of the run lasted a total of 54 hours at steady state conditions. The average TFAI conversion was 64.8 mole %, and the selectivity of CF₃I and the two major impurities, HFC23 and TFAF, was 99.5,% 0.42% and 0.06% on average, respectively.

The pressure was then increased to 220 psig and the reactor temperature was reduced to 350° C. This portion of the pair was named HP220 Run #1. During the 52 hours of this portion of the run, the average TFAI conversion was 70.1% mole %, HFC23 selectivity increased to 0.50%, and the TFAF selectivity decreased to 0.03% on average. The CF₃I conversion had an average value of 99.5%. The data for the LP and HP portions of the first experimental pair are comparable.

A further LP/HP experimental pair was run using the same procedure.

The LP portion of the pair was run for a total of 44 hours at steady state conditions. The average TFAI conversion was 66.31 mole %, and the selectivity of CF₃I and the two major impurities, HFC23 and TFAF, was 99.5,% 0.47% and 0.05% on average, respectively.

The pressure was then increased to 220 psig and the reactor temperature was reduced to 350° C. This portion of the pair was named HP220 Run #2. During the 56 hours of this portion of the run, the average TFAI conversion was 67.4% mole %, HFC23 selectivity decreased to 0.50%, and the TFAF selectivity decreased to 0.04% on average. The CF₃I conversion had an average value of 99.5%. The data for the LP and HP portions of the second experimental pair are comparable.

The operating conditions, average TFAI conversion and product selectivities are shown below in Table 30, in which PFP is pentafluoropropanone, and the low- and high-pressure runs are 25 psig and 220 psig, respectively.

TABLE 30

| | | Feed | | Produc- | | | | | | | TFAI |
|---|---|---|---|---|---|---|---|---|---|---|---|
| | | rate | | tivity | TFAF | R23 | PFP | CF₃I | C₂F₅I | CHF₂I | Conv. |
| T | P | lb/ | | lb/hr/ | Mol | Mol | Mol | Mol | Mol | Mol | Mol |
| ° C. | psig | hr | CTs | ft² | % | % | % | % | % | % | % |
| 410 | 25 | 0.25 | 2.04 | 666 | 0.06 | 0.42 | 0.01 | 99.51 | 0.00 | 0.00 | 64.81 |
| 350 | 220 | 0.25 | 13.2 | 721 | 0.03 | 0.50 | 0.01 | 99.45 | 0.00 | 0.00 | 70.11 |
| 410 | 25 | 0.25 | 2.04 | 678 | 0.05 | 0.47 | 0.01 | 99.49 | 0.00 | 0.00 | 66.31 |
| 350 | 220 | 0.25 | 13.3 | 692 | 0.04 | 0.46 | 0.00 | 99.50 | 0.00 | 0.00 | 67.35 |

### Example 29: Laboratory-Scale Purification of CF₃I

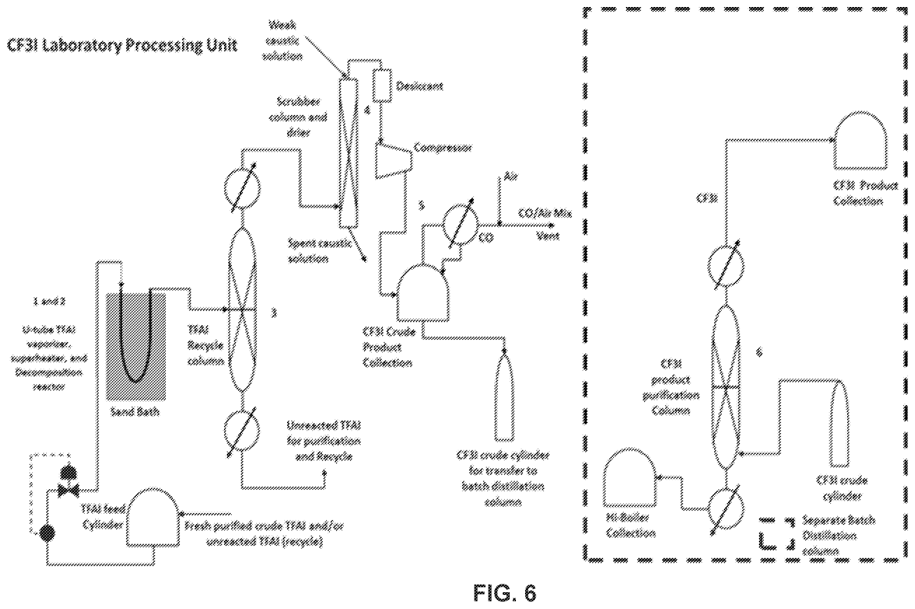

This example demonstrates the operation of a laboratory processing unit that starts with purified TFAI and produces an unreacted TFAI stream for recycle, a purified CF3I product, and a CO waste stream. The processing unit is shown in FIG. 6.

The laboratory processing unit consists of the following steps/unit operations: (1) vaporization of TFAI, (2) gas phase pyrolysis of TFAI, (3) separation of unconverted TFAI for recycle (recycle column reboiler stream is sent to Step 2 TFAI purification column), (4) acid removal, (5) CO removal, and (6) distillation of crude CF₃I.

In step (1), during the vaporization/superheating of TFAI, TFAI is fed at a predetermined flow rate from a storage cylinder to individual vaporizers/superheaters and are heated to a predetermined temperature. The superheated TFAI is then fed to a preheated reactor for reaction.

In step (2), the pyrolysis reaction of TFAI occurs in a gas phase reactor equipped with an electric heating element. Conversion is a function of electric heating element surface (skin) temperature and contact time. Contact time is determined by reactor volume, TFAI feed rate, pressure, and temperature.

In step (3), TFAI is separated from CF₃I and CO, and TFAI is recycled. CF₃I and CO crude reaction products are separated as the overhead stream in a TFAI recycle column. The column bottom stream consisting predominately of TFAI is sent to a TFAI purification column before being recycled back to the TFAI pyrolysis reactor.

In step (4), the overhead stream of the recycle column is scrubbed at low pressure. The CF₃I and CO crude reaction products exiting the top of the TFAI recycle column are passed through a caustic scrubber to remove residual acid and then dried using a desiccant.

In step (5), the stream is compressed, and CO is removed. The acid-free crude CF₃I and CO are compressed into a CF₃I/CO separator, where CO is vented and CF₃I is collected into a product collection cylinder with the help of a CF₃I reflux condenser.

In step (6), the CF₃I product is purified. A batch distillation is employed to purify crude product into refrigerant grade CF₃I.

The CF₃I laboratory processing unit was run continuously for a total of 708 hours and was called Campaigns 16 and 17 (C16 and C17). The purpose of the campaigns was to demonstrate the long-term stability of the process at a low pressure operating condition. The TFAI feedstocks for C16 and C17 were purified crude TFAI produced by the reaction of trifluoroacetyl chloride (TFAC) and hydrogen iodide (HI). The purity of the feedstocks was determined by gas chromatography and was 99.1% for C16 and 99.2% for C17.

A total of 79 lbs of TFAI was fed to the unit during C16 and 104.5 lbs was fed during C17. The reaction was run at what was called ‘baseline conditions’ for the entire time: a TFAI feed rate of 0.25 lb/hr, a pressure of 25 psig, and an electric heater element temperature of 390±1° C. This provided a contact time (CT) of 2.10 s.

The average TFAI conversion over the 708 on-stream hours of operation was 70.4 mol %, and the selectivity of CF₃I and the two major impurities, HFC23 and TFAF, was 99.4%, 0.51%, and 0.07% on average, respectively. Both the conversion and selectivities were relatively steady throughout the campaigns. The campaigns served their purpose in accumulating more on-stream time to demonstrate the long-term stability of the process at baseline operating conditions. A summary of the average operating conditions and results of the two campaigns is shown below in Table 31, wherein PFP is pentafluoropropanone. No formation of CHF₂I was observed.

TABLE 31

| | | Feed | | Produc- | | | | | | TFAI |
|---|---|---|---|---|---|---|---|---|---|---|
| | | rate | | tivity | TFAF | R23 | PFP | CF₃I | C₂F₅I | Conv. |
| T | P | lb/ | | lb/hr/ | Mol | Mol | Mol | Mol | Mol | Mol |
| ° C. | psig | hr | CTs | ft² | % | % | % | % | % | % |
| 350 | 25 | 0.25 | 2.1 | 715 | 0.067 | 0.509 | 0.002 | 99.4 | 0.006 | 70.4 |
| 410 | 25 | 0.25 | 2.04 | 678 | 0.05 | 0.47 | 0.01 | 99.49 | 0.00 | 66.31 |
| 350 | 220 | 0.25 | 13.3 | 692 | 0.04 | 0.46 | 0.00 | 99.50 | 0.00 | 67.35 |

The reactor effluent was fed to a continuous distillation column (as described below) where the CF₃I, CO, and low boiling impurities were continuously taken out of the top of the condenser and fed to a scrubber that contained a weak NaOH solution to neutralize any acidic low boiling impurities. The scrubber equipment is described below. Before and after scrubber samples were taken to verify and observe the disappearance of the acidic impurities. The operating conditions of the TFAI recycle column are shown in Table 32.

TABLE 32

| Component/Description | Conditions |
|---|---|
| Reboiler | 10 gallon, jacketed |
| Heat source | Regulated 150# steam |
| Column size | 2″ inner diameter x 8′ length |
| Packing | 1/4″ monel Propak |
| Condenser size | 12 ft² |
| Condenser coolant | −20° C. Ethanol |
| Chiller | |
| Pressure | 24.0-24.5 psia |
| Pressure change | 17-20″ H₂O |
| O/H draw-off rate | 0.15-0.18 lb/hr |
| Column O/H temperature | 2-3.5° C. |
| Reboiler temperature | 65-70° C. |

Table 33 shows the components and conditions for the scrubber.

TABLE 33

| | |
|---|---|
| Component | Conditions |
| Column | PFA-lined carbon steel |
| Column packing | 3.4″ polypropylene balls (dump packing) |
| Scrubber tanks/sumps × | 30 gallon fluoropolymer-lined steel |
| 2 in parallel | |
| Scrubber solution | Fresh solution, 0.5% KOH |
| Circulation pump | Easter Centrichem centrifugal pump |

Tables 34 and 35 show the compositions of crude CF₃I before and after the scrubber, respectively. All values are shown in GC area 00 The scrubber was originally charged with 0.5 wt. %0 sodium hydroxide (NaOH).

TABLE 34

| FC-116 | | | | | Methyl | | | |
|---|---|---|---|---|---|---|---|---|
| (CF₃CF₃) | TFAF* | R23 | TFAC* | CF₃I | propane | C₂F₅I | CH₃I | other |
| 0.0000 | 0.1577 | 0.1371 | 0.8484 | 98.6999 | 0.0154 | 0.0000 | 0.0004 | 0.1411 |
| 0.0000 | 0.1283 | 0.1360 | 0.5371 | 99.1924 | 0.0034 | 0.0000 | 0.0003 | 0.0539 |
| 0.0010 | 0.1152 | 0.1322 | 0.4904 | 99.3054 | 0.0041 | 0.0000 | 0.0003 | 0.0028 |
| 0.0000 | 0.0985 | 0.1811 | 0.1659 | 99.5888 | 0.0051 | 0.0000 | 0.0000 | 0.0120 |
| 0.0100 | 0.1032 | 0.1809 | 0.1595 | 99.6011 | 0.0055 | 0.0000 | 0.0001 | 0.0001 |
| 0.0000 | 0.1094 | 0.1702 | 0.0983 | 99.6734 | 0.0000 | 0.0000 | 0.0001 | 0.0000 |

*Acid impurity

TABLE 35

| | | | | | Methyl | | | |
|---|---|---|---|---|---|---|---|---|
| FC-116 | TFAF* | R23 | TFAC* | CF₃I | propane | C₂F₅I | CH₃I | other |
| 0.0000 | ND | 0.0751 | ND | 99.0968 | 0.0031 | 0.0000 | 0.0002 | 0.0331 |
| 0.0000 | ND | 0.0337 | ND | 99.7459 | 0.0035 | 0.0000 | 0.0003 | 0.0217 |
| 0.0000 | ND | 0.0380 | ND | 99.7441 | 0.0033 | 0.0000 | 0.0002 | 0.0191 |
| 0.0000 | ND | 0.1855 | ND | 99.8589 | 0.0068 | 0.0000 | 0.0001 | 0.0000 |
| 0.0000 | ND | 0.1796 | ND | 99.8420 | 0.0062 | 0.0000 | 0.0001 | 0.0234 |
| 0.0000 | ND | 0.1794 | ND | 99.8715 | 0.0000 | 0.0000 | 0.0003 | 0.0001 |

*Acid impurity

After exiting the scrubber column, the stream was fed to a column packed with Drierite (calcium sulfate) desiccant to remove moisture. The dry CF₃I/CO crude stream was then compressed to about 200 psig and fed to a cooled product collection cylinder (PCC) with an attached reflux condenser that was subcooled by liquid nitrogen. Carbon monoxide (and some CF₃I) was continuously vented from the system using a back-pressure regulator. Seventy-three pounds of CF₃I was collected in the PCC during the campaigns. The typical quality of the CF₃I crude material that has been collected in the PCC over multiple campaigns can be seen below in Table 36.

TABLE 36

| Peak # | RT (min) | Component | Area FID | Area % FID |
|---|---|---|---|---|
| 1 | 7.411 | Hexafluoroethane | 25202 | 0.0007 |
| 2 | 7.606 | Trifluoromethane | 5716240 | 0.1672 |
| 3 | 7.957 | C₂F₅H | 13873 | 0.0004 |
| 4 | 9.36 | G-1234 isomer | 15453 | 0.0005 |
| 5 | 9.487 | Propene | 16276 | 0.0005 |
| 6 | 9.633 | Unknown | 15895 | 0.0005 |
| 7 | 11.094 | CF₃I | 3.41E+09 | 99.7840 |
| 8 | 11.611 | Methyl propane | 1328765 | 0.0389 |
| 9 | 12.332 | G-133a | 46576 | 0.0014 |
| 10 | 13.375 | CH₃I | 205351 | 0.0060 |

Some of this material was further distilled in the two-liter R12 high pressure batch distillation column described below. About 2150 grams of the CF₃I crude material was charged and a 102 gram lights cut was taken after venting off noncondensibles. 920 grams of the main cut was collected and called Main Cut #1. An additional 534 grams of distillate was collected and called Main Cut #2. The reboiler residue was analyzed by lab GC after the completion of the distillation. Both Main Cuts were about 99.99% pure, much greater than the 99.5% CF₃I manufacturing spec. The nonvolatile residue, acidity, and moisture analysis can be found in the table below. The moisture level was 10× higher than the refrigeration grade specification, but treating the material with drierite desiccant reduced the moisture level to 4 ppm, which is lower than the 10 ppm spec. The reboiler had about 38% of hi-boilers (relative to CF₃I) left over which shows that there are no problematic impurities that are produced by the 3-step process starting with H₂ and I₂ (to produce HI) and TFAC.

The components and descriptions of conditions of the final CF₃I batch distillation equipment is shown below in Table 37.

TABLE 37

| Component/Description | Conditions |
|---|---|
| Reboiler | 2 L electrically traced |
| Reboiler MOC | PTFE lined |
| Heat source | 110 V (variac) |
| Column size | 1″ inner diameter × 6′ length |
| Theoretical stages | 60 (estimated) |
| Packing | Nichrome springs |
| Condenser coolant | −30 to −25° C. ethanol circulating chiller |
| Pressure | 40-50 psig |
| Pressure change | 4-6″ H₂O |
| O/H draw-off rate (lites cut) | 1-100 g/hr |
| O/H draw-off rate (main cut) | 1-100 g/hr |
| Column O/H temp. | 2-7° C. (pressure dependent) |

Table 38 shows the GC analysis of the various cuts and reboiler residue from a laboratory processing unit produced during the purification of crude CF₃I by batch distillation.

TABLE 38

| | | | | | | Methyl | | | | |
|---|---|---|---|---|---|---|---|---|---|---|
| Description | TFAF | R23 | TFAC | C₂F₅H | CF₃I | propane | 133a | C₂F₅I | CH₃I | other |
| Lites cut | ND | 0.0654 | ND | 0.005 | 99.9197 | 0.0059 | 0.0039 | ND | 0.0001 | 0.0 |
| Main cut 1 | ND | 0.0017 | ND | 0.0029 | 99.9915 | 0.0038 | ND | ND | 0.0001 | 0.0 |
| Main cut 2 | ND | 0.0012 | ND | 0.0015 | 99.9901 | 0.0071 | ND | ND | 0.00005 | 0.0 |
| Reboiler | ND | ND | ND | ND | 61.95 | 0.0464 | ND | ND | ND | 38.0036 |
| residue | | | | | | | | | | |

Table 39 shows additional analysis of Main Cut 1 from the purification of crude CF₃I from a laboratory processing unit via batch distillation.

TABLE 39

| | | Moisture | Moisture | |
|---|---|---|---|---|
| | Acidity | before | after | |
| NVR | ppm as | drying | drying* | |
| ppm | HCl | ppm | ppm | Comment |
| <100 | <1 | <10 | <10 | CF₃I refrigerant grade |
| | | | | manufacturing spec |
| 1.3 | 0.3 | 103.2 | 4 | |

*Drierite used as dessicant

The unreacted TFAI and high-boiling impurities were allowed to accumulate in the reboiler for the entire 708 hours of on-stream time. This material was combined with other accumulated unreacted TFAI material from other previous and subsequent campaigns until the reboiler level was about 80%. A total of 175 lbs of unreacted TFAI was drained from the reboiler. Next, the material was batch distilled to remove I₂ and high-boiling impurities. The batch distillation column used is described below. After charging the distillation column reboiler, the distillation was started, and after venting off the non-condensables and low-boiling impurities, such as CF₃I. 136.8 lbs of distillate was collected in the Product Collection Cylinder (PCC). The distillation was stopped after the reboiler temperature had increased to >10° C. above the column temperatures as per the normal procedure. A gas chromatography (GC) analysis of the composite TFAI collected in the PCC determined that the purity of the material was 99.0%. The material was transferred to the feed cylinder of the CF₃I laboratory processing unit prior to starting Campaign 22 (C22) to demonstrate that unreacted TFAI can be recycled.

The conditions for the TFAI batch column are shown below in Table 40.

TABLE 40

| Component/Description | Conditions |
|---|---|
| Reboiler | 10 gallon jacketed |
| Condenser size | 8 ft2 |
| Heat source | 30# steam |
| Column size | 2″ inner diameter × 8′ length |
| Theoretical stages | 35 (estimated) |
| Packing | 1/4″ monel Propak |
| Condenser coolant | −20 to −10° C. ethanol |
| | NESLAB chiller |
| Pressure | −4.0 to 30 psig |
| Pressure change | 25-30″ H₂O |
| O/H draw-off rate (lites venting) | 0.1-0.5 lb/hr (to house scrubber) |
| O/H draw-off rate (main cut) | 0.5-1.0 lb/hr |
| Column O/H temp. (lites venting) | 0-10° C. (pressure dependent) |
| Column O/H temp. (main cut) | 28-38° C. (pressure dependent) |
| Reboiler temp. (lites venting) | 0-10° C. (pressure dependent) |
| Reboiler temp. (main cut) | 28-65° C. (pressure dependent) |

After the purified, unreacted TFAI was charged to the feed cylinder for the CF₃I laboratory processing unit the reaction was started up at high pressure conditions of 220 psig, 0.25 lb/hr TFAI feed, and 340° C. and and brought to steady operating conditions and run there continuously for about 100 hours while reactor exit samples were taken at regular 4 hour intervals and analyzed by gas chromatography (GC). This run was called Campaign 22 (C22).

The average TFAI conversion was 68.4% mole %, HFC-23 selectivity was 0.28% and the TFAF selectivity was 0.07% on average, and the CF₃I conversion was at an average value of 99.6%. These results showed that unreacted TFAI can be successfully recycled to the TFAI decomposition reactor without loss of CF₃I selectivity. Table 41 is a summary of the C22 average operating conditions and results. In the PFP is pentafluoropropanone, and the selectivities for TFAF, R23, PFP, CF₃I, C₂F₅I, and CHF₂I are shown in mole percent. The conversion of TFAI (“TFAI Conv.”) is shown in mole percent. The feedstock was unreacted TFAI recycle. The on-stream time was 100 hours.

TABLE 41

| | | Feed | | Produc- | | | | | | | |
|---|---|---|---|---|---|---|---|---|---|---|---|
| T | P | rate | | tivity | | | | | | | TFAI |
| ° C. | psig | lb/hr | CTs | lb/ht/ft² | TFAF | R23 | PFP | CF₃I | C₂F₅I | CHF₂I | Conv. |
| 340 ± 1 | 220 | 0.25 | 13.2 | 704 | 0.07 | 0.28 | 0.01 | 99.64 | 0.0 | 0.0 | 68.41 |

### ASPECTS

Aspect 1 is a process for producing trifluoroiodomethane (CF₃I). The process includes (a) providing a first reactant stream comprising hydrogen iodide (HI); (b) reacting the first reactant stream with a second reactant stream comprising trifluoroacetyl chloride (TFAC) to produce an intermediate product stream comprising trifluoroacetyl iodide (TFAI); and (c) reacting the intermediate product stream to produce a final product stream comprising trifluoroiodomethane (CF₃I).

Aspect 2 is the process of Aspect 1, wherein hydrogen (H₂) and iodine (I₂) are reacted to produce the first reactant stream comprising hydrogen iodide (HI).

Aspect 3 is the process of either Aspect 1 or Aspect 2, further including at least one of: a temperature from about 150° C. to about 600° C.; a pressure from about 0 psig to about 600 psig; a mole ratio of hydrogen (H₂) to iodine (I₂) of about 1.0 to about 10.0; and a catalyst.

Aspect 4 is the process of any of Aspects 1-3 wherein the first product stream further comprises unreacted iodine (I₂) and unreacted hydrogen, both of which are recycled to the reaction step.

Aspect 5 is the process of any of Aspects 1-4, wherein the process comprises a first catalyst, and the first catalyst comprises at least one catalyst selected from the group of nickel, nickel iodide (NiI₂), cobalt, iron, nickel oxide (NiO), cobalt oxide, and iron oxide, cobalt(II) iodide (CoI₂), iron(II) iodide (FeI₂), and iron(III) iodide (FeI₃).

Aspect 6 is the process of any of Aspects 1-5, wherein the second reactant stream further comprises sulfur dioxide (SO₂) and the process further comprises, prior to step (b), the additional step of: (i) removing sulfur dioxide (SO₂) by forming an azeotrope or azeotrope-like composition of trifluoroacetyl chloride (TFAC) and sulfur dioxide (SO₂) and feeding the composition into a distillation column; or (ii) contacting a mixture of trifluoroacetyl chloride (TFAC) and sulfur dioxide (SO₂) with at least one solid adsorbent to remove sulfur dioxide (SO₂) from the mixture of trifluoroacetyl chloride (TFAC) and sulfur dioxide (SO₂).

Aspect 7 is the process of any of Aspects 1-6, wherein step (b) further comprises at least one of the following: a temperature from about 25° C. to about 180° C.; a pressure from about 0 to about 225 psig; a mole ratio of trifluoroacetyl chloride (TFAC) to hydrogen iodide (HI) from about 2.0:1.0 to about 0.02:1.0; and a catalyst.

Aspect 8 is the process of any of Aspects 1-7, wherein, in step (b), the second reactant stream comprises a plurality of components wherein the sum of TFAC and HI comprises at least 99 wt. %; sulfur dioxide (SO₂) is present in an amount of not more than 250 ppm; the sum of iodine and HI₃ is no more than 2000 ppm; iodohydrocarbons comprising one or more of iodomethane, iodoethane, iodopropane, iodobutane, tert-butyl iodide, and diiodopropane are present in an amount of not more than 500 ppm; hydrogen is present in an amount of not more than 500 ppm; and CF₃I is present in an amount of not more than 5000 ppm.

Aspect 9 is the process of any of Aspects 1-8, wherein step (b) further comprises a catalyst and the catalyst comprises at least one catalyst selected from the group of activated carbon and silica carbide.

Aspect 10 is the process of any of Aspects 1-9, wherein the intermediate product stream further comprises unreacted trifluoroacetyl chloride (TFAC) and the process further comprises the additional steps of: (i) separating unreacted trifluoroacetyl chloride (TFAC) from the intermediate product stream; and (ii) returning the separated trifluoroacetyl chloride to the reactant stream.

Aspect 11 is the process of any of Aspects 1-10, wherein the intermediate product stream further comprises at least one of trifluoroacetyl chloride (TFAC), hydrogen iodide (HI), hydrogen chloride (HCl), trifluoroacetic acid (TFA), trifluoroiodomethane (CF₃I), an iodine-containing species and carbon monoxide (CO), and step (b) further comprises purifying the intermediate product stream to obtain a purified intermediate product stream having a concentration of trifluoroacetyl iodide (TFAI) of greater than about 99%.

Aspect 12 is the process of Aspect 11, wherein purifying the intermediate product stream further comprises: (i) feeding the intermediate product stream into a first distillation column to obtain a first overhead stream comprising at least one of trifluoroacetyl chloride (TFAC), hydrogen iodide (HI), hydrogen chloride (HCl), trifluoroiodomethane (CF₃I), and carbon monoxide (CO) and first a bottoms stream comprising trifluoroacetyl iodide (TFAI), trifluoroacetic acid (TFA), and iodine-containing species; and (ii) feeding the first overhead stream to a second distillation column to obtain a second overhead stream comprising hydrogen chloride (HCl) and a second bottoms stream comprising hydrogen iodine (HI) and trifluoroacetyl chloride (TFAC).

Aspect 13 is the process Aspect 11, wherein purifying the intermediate product stream further comprises: (i) feeding the intermediate product stream into a first distillation column to obtain a first overhead stream comprising hydrogen chloride (HCl) and first a bottoms stream comprising trifluoroacetyl iodide (TFAI), hydrogen iodide (HI) and trifluoroacetyl chloride (TFAC); and (ii) feeding the first bottoms stream to a second distillation column to obtain a second overhead stream comprising hydrogen iodide (HI) and trifluoroacetyl chloride (TFAC) and a second bottoms stream comprising trifluoroacetyl iodide (TFAI) wherein the second distillation column is operated at a pressure lower than a pressure of the first distillation column.

Aspect 14 is the process of any of Aspects 11-13, wherein purifying the intermediate product stream is carried out at a temperature lower than about 150° C.

Aspect 15 is the process of any of Aspects 1-14, further comprising removing at least one iodine-containing species from a stream comprising trifluoroacetyl iodide (TFAI) or trifluoroiodomethane (CF₃I) by contacting the stream comprising trifluoroacetyl iodide (TFAI) or trifluoroiodomethane (CF₃I) with carbonaceous materials to remove at least one of hydrogen iodide (HI), hydrogen triiodide (HI₃) and iodine (I₂) from the stream.

Aspect 16 is the process of Aspect 12, further comprising: removing at least one iodine-containing species from the first bottoms stream by adding a solvent to the first bottoms stream to provide a stream comprising the solvent and the first bottoms stream; passing the stream comprising the solvent and the first bottoms stream to a second column to provide a second overhead product and a second bottoms product; and passing the second bottoms product to a third column to provide a third overhead product and a third bottoms product, wherein the third bottoms product comprises iodine.

Aspect 17 is the process of Aspect 16, wherein the solvent comprises toluene.

Aspect 18 is the process of either Aspect 16 or Aspect 17, wherein the solvent is recycled and added to the first bottoms stream.

Aspect 19 is the process of any of Aspects 1-18, wherein step (c) further comprises at least one of the following: a reaction temperature from about 300° C. to about 450° C.; a pressure from about 25 to about 300 psig; and the reaction occurs in an immersion type electric heater reactor, a shell and tube reactor or an impedance heater type reactor.

Aspect 20 is the process of any of Aspects 1-19, wherein the final product stream further comprises at least one of carbon monoxide (CO), carbon dioxide (CO₂), R23 (CH₃F), R13 (CClF₃), trifluoroacetyl fluoride (TFAF), trifluoroacetic acid (TFA), pentafluoropropanone, 133a (2-chloro-1,1,1-trifluoroethane), pentafluoroiodoethane (C₂F5I), iodine (I₂) or TFAI, and step (c) further comprises purifying the final product stream to obtain a purified final product stream having a concentration of trifluoroiodomethane (CF₃I) of greater than about 99%.

Aspect 21 is the process of any of Aspects 1-20, further comprising removing at least one iodine-containing species from the final product stream by adding a solvent to the final product stream to provide a stream comprising the solvent and the final product stream; passing the stream comprising the solvent and the final product stream to a first column to provide a first overhead product and a first bottoms product; and passing the first bottoms product to a second column to provide a second overhead product and a second bottoms product, wherein the second bottoms product comprises iodine.

Aspect 22 is the process of Aspect 21, wherein the solvent comprises toluene.

Aspect 23 is the process of either Aspect 21 or Aspect 22 wherein the solvent is recycled and added to the first bottoms stream.

Aspect 24 is the process of Aspect 20, wherein purifying the final product stream further comprises: (i) providing the final product stream to a first distillation column to obtain an overhead stream comprising trifluoroiodomethane (CF₃I), carbon monoxide (CO), and low-boiling impurities, and a bottoms stream comprising trifluoroacetyl iodide (TFAI) and high-boiling impurities; and (ii) providing the overhead stream comprising trifluoroiodomethane (CF₃I), carbon monoxide (CO), and low-boiling impurities combined with a hydrogen chloride (HCl) stream to a second distillation column to obtain an overhead stream of the second column comprising carbon monoxide (CO) and hydrogen chloride (HCl), and a bottoms stream of the second column comprising trifluoroiodomethane (CF₃I), impurities, and residual acid.

Aspect 25 is the process of Aspect 24, further comprising (i) providing the bottoms stream of the second column to a scrubbing system to remove residual acid to obtain a stream comprising trifluoroiodomethane, impurities, and water; and (ii) drying the stream comprising trifluoroiodomethane, impurities, and water to remove water to obtain a dried stream comprising trifluoroiodomethane, impurities.

Aspect 26 is the process of Aspect 25, wherein the drying step further comprises exposing the stream comprising trifluoroiodomethane, impurities, and water to a desiccant selected from the group consisting of at least one of molecular sieves, anhydrous calcium chloride, anhydrous calcium sulfate, concentrated sulfuric acid, silica gel, activated charcoal, zeolites, and combinations of the foregoing.

Aspect 27 is the process of Aspect 25 or Aspect 26, wherein the drying step comprises contacting the stream comprising trifluoroiodomethane, impurities, with a concentrated sulfuric acid solution.

Aspect 28 is the process any of Aspects 25-27, further comprising: (i) providing the dried stream comprising trifluoroiodomethane, low-boiling impurities, and high-boiling impurities to a third distillation column to provide a third overhead product stream comprising low-boiling impurities and a third bottoms product stream comprising trifluoroiodomethane and high-boiling impurities; and (ii) providing the third bottoms product stream to a fourth distillation column to provide a fourth bottoms product stream comprising high-boiling impurities and a fourth overhead product stream comprising purified trifluoromethane (CF₃I).

What is claimed is:

1. A process for producing trifluoroiodomethane (CF₃I), the process comprising:
(a) providing a first reactant stream comprising hydrogen iodide (HI);
(b) reacting the first reactant stream with a second reactant stream comprising trifluoroacetyl chloride (TFAC) to produce an intermediate product stream comprising trifluoroacetyl iodide (TFAI); and
(c) reacting the intermediate product stream to produce a final product stream comprising trifluoroiodomethane (CF₃I).

2. The process of claim 1, wherein hydrogen (H₂) and iodine (I₂) are reacted to produce the first reactant stream comprising hydrogen iodide (HI).

3. The process of claim 2, further comprising at least one of:
a temperature from about 150° C. to about 600° C.;
a pressure from about 0 psig to about 600 psig;
a mole ratio of hydrogen (H₂) to iodine (I₂) of about 1.0 to about 10.0; and
a catalyst.

4. The process of claim 1, wherein the first product stream further comprises unreacted iodine (I₂) and unreacted hydrogen, both of which are recycled to the reaction step

5. The process of claim 2, wherein the process comprises a first catalyst, and the first catalyst comprises at least one catalyst selected from the group of nickel, nickel iodide (NiI₂), cobalt, iron, nickel oxide (NiO), cobalt oxide, and iron oxide, cobalt(II) iodide (CoI₂), iron(II) iodide (FeI₂), and iron(III) iodide (FeI₃).

6. The process of claim 1, wherein the second reactant stream further comprises sulfur dioxide (SO₂) and the process further comprises, prior to step (b), the additional step of:
(i) removing sulfur dioxide (SO₂) by forming an azeotrope or azeotrope-like composition of trifluoroacetyl chloride (TFAC) and sulfur dioxide (SO₂) and feeding the composition into a distillation column; or
(ii) contacting a mixture of trifluoroacetyl chloride (TFAC) and sulfur dioxide (SO₂) with at least one solid adsorbent to remove sulfur dioxide (SO₂) from the mixture of trifluoroacetyl chloride (TFAC) and sulfur dioxide (SO₂).

7. The process of claim 1, wherein step (b) further comprises at least one of the following:
a temperature from about 25° C. to about 180° C.;
a pressure from about 0 to about 225 psig;
a mole ratio of trifluoroacetyl chloride (TFAC) to hydrogen iodide (HI) from about 2.0:1.0 to about 0.02:1.0; and
a catalyst.

8. The process of claim 1, wherein, in step (b), the second reactant stream comprises:
a plurality of components wherein the sum of TFAC and HI comprises at least 99 wt. %;
sulfur dioxide (SO₂) is present in an amount of not more than 250 ppm;
the sum of iodine and HI₃ is no more than 2000 ppm;
iodohydrocarbons comprising one or more of iodomethane, iodoethane, iodopropane, iodobutane, tert-butyl iodide, and diiodopropane are present in an amount of not more than 500 ppm;
hydrogen is present in an amount of not more than 500 ppm; and
CF₃I is present in an amount of not more than 5000 ppm.

9. The process of claim 1, wherein step (b) further comprises a catalyst and the catalyst comprises at least one catalyst selected from the group of activated carbon and silica carbide.

10. The process of claim 1, wherein the intermediate product stream further comprises unreacted trifluoroacetyl chloride (TFAC) and the process further comprises the additional steps of:
(i) separating unreacted trifluoroacetyl chloride (TFAC) from the intermediate product stream; and
(ii) returning the separated trifluoroacetyl chloride to the reactant stream.

11. The process of claim 1, wherein the intermediate product stream further comprises at least one of trifluoroacetyl chloride (TFAC), hydrogen iodide (HI), hydrogen chloride (HCl), trifluoroacetic acid (TFA), trifluoroiodomethane (CF₃I), an iodine-containing species and carbon monoxide (CO), and step (b) further comprises purifying the intermediate product stream to obtain a purified intermediate product stream having a concentration of trifluoroacetyl iodide (TFAI) of greater than about 99%.

12. The process of claim 11, wherein purifying the intermediate product stream further comprises:
(i) feeding the intermediate product stream into a first distillation column to obtain a first overhead stream comprising at least one of trifluoroacetyl chloride (TFAC), hydrogen iodide (HI), hydrogen chloride (HCl), trifluoroiodomethane (CF₃I), and carbon monoxide (CO) and first a bottoms stream comprising trifluoroacetyl iodide (TFAI), trifluoroacetic acid (TFA), and iodine-containing species; and
(ii) feeding the first overhead stream to a second distillation column to obtain a second overhead stream comprising hydrogen chloride (HCl) and a second bottoms stream comprising hydrogen iodine (HI) and trifluoroacetyl chloride (TFAC).

13. The process of claim 11, wherein purifying the intermediate product stream further comprises:
(i) feeding the intermediate product stream into a first distillation column to obtain a first overhead stream comprising hydrogen chloride (HCl) and first a bottoms stream comprising trifluoroacetyl iodide (TFAI), hydrogen iodide (HI) and trifluoroacetyl chloride (TFAC); and
(ii) feeding the first bottoms stream to a second distillation column to obtain a second overhead stream comprising hydrogen iodide (HI) and trifluoroacetyl chloride (TFAC) and a second bottoms stream comprising trifluoroacetyl iodide (TFAI) wherein the second distillation column is operated at a pressure lower than a pressure of the first distillation column.

14. The process of claim 11, wherein purifying the intermediate product stream is carried out at a temperature lower than about 150° C.

15. The process of claim 1, further comprising removing at least one iodine-containing species from a stream comprising trifluoroacetyl iodide (TFAI) or trifluoroiodomethane (CF₃I) by contacting the stream comprising trifluoroacetyl iodide (TFAI) or trifluoroiodomethane (CF₃I) with carbonaceous materials to remove at least one of hydrogen iodide (HI), hydrogen triiodide (HI₃) and iodine (I₂) from the stream.
